SEQUENCE LISTING (770 sequences)

SEQ ID NO: 1 - DNA, 50 bp, Artificial Sequence
  Description of Artificial Sequence/note = Synthetic Construct
gagttcgagaccagcctgggcaacatagcgagaccccgtctctaaaaaaa

SEQ ID NO: 2 - DNA, 50 bp, Artificial Sequence
  Description of Artificial Sequence/note = Synthetic Construct
ggagttcgagaccagcctgggcaacatagcgagaccccgtctctaaaaaa

SEQ ID NO: 3 - DNA, 50 bp, Artificial Sequence; identical to SEQ ID NO: 2
  Description of Artificial Sequence/note = Synthetic Construct

SEQ ID NO: 4 - DNA, 50 bp, Artificial Sequence
  Description of Artificial Sequence/note = Synthetic Construct
gaggcaggaggatcgcttgagcccaggagttcgaggctgcagtgagctat

SEQ ID NO: 5 - DNA, 50 bp, Artificial Sequence
  Description of Artificial Sequence/note = Synthetic Construct
ggagttcgagaccagcctgggcaacatggtgaaaccccgtctctacaaaa

SEQ ID NO: 6 - DNA, 50 bp, Artificial Sequence
  Description of Artificial Sequence/note = Synthetic Construct
tcacgaggtcaagagatcgagaccatcctggccaacatggtgaaaccccg

SEQ ID NO: 7 - DNA, 50 bp, Artificial Sequence
  Description of Artificial Sequence/note = Synthetic Construct
ccaacatggtgaaaccccgtctctactaaaaatacaaaaattagccgggc

SEQ ID NO: 8 - DNA, 50 bp, Artificial Sequence
  Description of Artificial Sequence/note = Synthetic Construct
ccagcctgaccaacatggagaaaccccgtctctactaaaaatacaaaaat

SEQ ID NO: 9 - DNA, 50 bp, Artificial Sequence
  Description of Artificial Sequence/note = Synthetic Construct
caacatggtgaaaccccgtctctactaaaaatacaaaaattagccgggcg

SEQ ID NO: 10 - DNA, 50 bp, Artificial Sequence; identical to SEQ ID NO: 7
  Description of Artificial Sequence/note = Synthetic Construct

SEQ ID NO: 11 - DNA, 50 bp, Artificial Sequence; identical to SEQ ID NO: 7
  Description of Artificial Sequence/note = Synthetic Construct

SEQ ID NO: 12 - DNA, 50 bp, Artificial Sequence
  Description of Artificial Sequence/note = Synthetic Construct
gagatcgagaccatcctggctaacacggtgaaaccccgtctctactaaaa

SEQ ID NO: 13 - DNA, 50 bp, Artificial Sequence
  Description of Artificial Sequence/note = Synthetic Construct
cgggcggatcacgaggtcaggagatcgagaccatcccggctaaaacggtg

SEQ ID NO: 14 - DNA, 50 bp, Artificial Sequence
  Description of Artificial Sequence/note = Synthetic Construct
gaaaccccgtctctactaaaactacaaaaaatagccgggcgtagtggcgg

SEQ ID NO: 15 - DNA, 50 bp, Artificial Sequence
  Description of Artificial Sequence/note = Synthetic Construct
agaccatcctggctaacaaggtgaaaccccgtctctactaaaaatacaaa

SEQ ID NO: 16 - DNA, 50 bp, Artificial Sequence; identical to SEQ ID NO: 15
  Description of Artificial Sequence/note = Synthetic Construct

SEQ ID NO: 17 - DNA, 50 bp, Artificial Sequence; identical to SEQ ID NO: 12
  Description of Artificial Sequence/note = Synthetic Construct

SEQ ID NO: 18 - DNA, 50 bp, Artificial Sequence
  Description of Artificial Sequence/note = Synthetic Construct
gagatcgagaccatcctggctaacaaggtgaaaccccgtctctactaaaa

SEQ ID NO: 19 - DNA, 50 bp, Artificial Sequence
  Description of Artificial Sequence/note = Synthetic Construct
cgcctgtagtcccagctactcggagaggctgaggcaggagaatggcgtga

SEQ ID NO: 20 - DNA, 50 bp, Artificial Sequence
  Description of Artificial Sequence/note = Synthetic Construct
accatcctggctaacacggtgaaaccccgtctctactaaaaatacaaaaa

SEQ ID NO: 21 - DNA, 50 bp, Artificial Sequence
  Description of Artificial Sequence/note = Synthetic Construct
atggatttgaggtttcctcccatctcctcattcggcggccctacgattaa

SEQ ID NO: 22 - DNA, 50 bp, Artificial Sequence
  Description of Artificial Sequence/note = Synthetic Construct
acggatttgaggtttcctcccatctcctcattcggcagccctacgattaa

SEQ ID NO: 23 - DNA, 50 bp, Artificial Sequence
  Description of Artificial Sequence/note = Synthetic Construct
ggcgtattgacttgctgtgtgcatcgggcaatgaacctattacggttaca

SEQ ID NO: 24 - DNA, 50 bp, Artificial Sequence; identical to SEQ ID NO: 12
  Description of Artificial Sequence/note = Synthetic Construct

SEQ ID NO: 25 - DNA, 50 bp, Artificial Sequence; identical to SEQ ID NO: 13
  Description of Artificial Sequence/note = Synthetic Construct

SEQ ID NO: 26 - DNA, 50 bp, Artificial Sequence; identical to SEQ ID NO: 12
  Description of Artificial Sequence/note = Synthetic Construct

SEQ ID NO: 27 - DNA, 50 bp, Artificial Sequence; identical to SEQ ID NO: 12
  Description of Artificial Sequence/note = Synthetic Construct

SEQ ID NO: 28 - DNA, 50 bp, Artificial Sequence; identical to SEQ ID NO: 20
  Description of Artificial Sequence/note = Synthetic Construct

SEQ ID NO: 29 - DNA, 50 bp, Artificial Sequence; identical to SEQ ID NO: 12
  Description of Artificial Sequence/note = Synthetic Construct

SEQ ID NO: 30 - DNA, 50 bp, Artificial Sequence; identical to SEQ ID NO: 12
  Description of Artificial Sequence/note = Synthetic Construct

SEQ ID NO: 31 - DNA, 50 bp, Artificial Sequence
  Description of Artificial Sequence/note = Synthetic Construct
gagatcgagaccatcctggctaacgcggtgaaaccccgcctctactaaaa

SEQ ID NO: 32 - DNA, 50 bp, Artificial Sequence
  Description of Artificial Sequence/note = Synthetic Construct
agatcgagaccatcctggctaacacggtgaaaccccgtctctactaaaaa

SEQ ID NO: 33 - DNA, 50 bp, Artificial Sequence; identical to SEQ ID NO: 32
  Description of Artificial Sequence/note = Synthetic Construct

SEQ ID NO: 34 - DNA, 50 bp, Artificial Sequence
  Description of Artificial Sequence/note = Synthetic Construct
gaccatcctggctaacacggtgaaaccccgtctctactaaaaatacaaaa

SEQ ID NO: 35 - DNA, 50 bp, Artificial Sequence
  Description of Artificial Sequence/note = Synthetic Construct
gatcgagaccatcctggctaacacagtgaaaccccgtctctactaaaaaa

SEQ ID NO: 36 - DNA, 50 bp, Artificial Sequence; identical to SEQ ID NO: 4
  Description of Artificial Sequence/note = Synthetic Construct

SEQ ID NO: 37 - DNA, 50 bp, Artificial Sequence
  Description of Artificial Sequence/note = Synthetic Construct
ggctctgccacttactagctgtgtgaccttgggcaagttacttaacctct

SEQ ID NO: 38 - DNA, 50 bp, Artificial Sequence
  Description of Artificial Sequence/note = Synthetic Construct
atcacatggacacaggaaggggaatatcacactctggggactgtggtggg

SEQ ID NO: 39 - DNA, 50 bp, Artificial Sequence
  Description of Artificial Sequence/note = Synthetic Construct
cctgtcggggggtggggggctaggggagggatagcattaggagaaatacc

SEQ ID NO: 40 - DNA, 50 bp, Artificial Sequence
  Description of Artificial Sequence/note = Synthetic Construct
tgggcttaatacctaggtgatgggatgatctgtgcagcaaaccaccatgg

SEQ ID NO: 41 - DNA, 50 bp, Artificial Sequence
  Description of Artificial Sequence/note = Synthetic Construct
tcgggtactatgcttattacctgggtgacgaaataatctgtacaccaaac

SEQ ID NO: 42 - DNA, 50 bp, Artificial Sequence
  Description of Artificial Sequence/note = Synthetic Construct
atctcagaaatcaccactaaagaacttattcatgtaaccaaacaccacct

SEQ ID NO: 43 - DNA, 50 bp, Artificial Sequence
  Description of Artificial Sequence/note = Synthetic Construct
aagtgggagctaagctatgggtacgcaaaggcatacagagtggtataatg

SEQ ID NO: 44 - DNA, 50 bp, Artificial Sequence
  Description of Artificial Sequence/note = Synthetic Construct
gggaagggtagtggggggttggtggggaggtggggatggttaatgggtac

SEQ ID NO: 45 - DNA, 50 bp, Artificial Sequence
  Description of Artificial Sequence/note = Synthetic Construct
atagggagaggttggttaatggatacaaaattacagctagataggaggaa

SEQ ID NO: 46 - DNA, 50 bp, Artificial Sequence
  Description of Artificial Sequence/note = Synthetic Construct
agatcttaagtgttctcaccacacacaaaaaaatggtaactatgtgaggt

SEQ ID NO: 47 - DNA, 50 bp, Artificial Sequence
  Description of Artificial Sequence/note = Synthetic Construct
ctgcacawgctctcttgcctgccgccatgtaagacgtgmctttgctcctc

SEQ ID NO: 48 - DNA, 50 bp, Artificial Sequence
  Description of Artificial Sequence/note = Synthetic Construct
tccccttggtgctgtcctcgtgatagtgagtgagttctcgtgagatctgg

SEQ ID NO: 49 - DNA, 50 bp, Artificial Sequence
  Description of Artificial Sequence/note = Synthetic Construct
gattaatggattaatgggttatcatgggagtgggactggtggctttataa

SEQ ID NO: 50 - DNA, 50 bp, Artificial Sequence
  Description of Artificial Sequence/note = Synthetic Construct
tgaggacacagtgagaaggcgccgtctacgaaccagggaatgagccctca

SEQ ID NO: 51 - DNA, 50 bp, Artificial Sequence
  Description of Artificial Sequence/note = Synthetic Construct
ggagaagacggccatctacaagccaaggagagaggcctcagaagaaacca

SEQ ID NO: 52 - DNA, 50 bp, Artificial Sequence
  Description of Artificial Sequence/note = Synthetic Construct
ccagcaaaccaccagaagctaggggagaggcatggaacagattctccctc

SEQ ID NO: 53 - DNA, 50 bp, Artificial Sequence
  Description of Artificial Sequence/note = Synthetic Construct
ggtcagagtcagagaaggagatgtgacgacggaagcagaggtcggagtga

SEQ ID NO: 54 - DNA, 50 bp, Artificial Sequence
  Description of Artificial Sequence/note = Synthetic Construct
gattccgtcttgncgncantcttgctgagagnctctcttgctggctttga

SEQ ID NO: 55 - DNA, 50 bp, Artificial Sequence
  Description of Artificial Sequence/note = Synthetic Construct
tgtagtcccctcccacattgaatagggctgacctgtgtgaccaatagaat

SEQ ID NO: 56 - DNA, 50 bp, Artificial Sequence
  Description of Artificial Sequence/note = Synthetic Construct
caagaggtgacttgggtgctgttaaaggcattcagttttaaaagggaagc

SEQ ID NO: 57 - DNA, 50 bp, Artificial Sequence
  Description of Artificial Sequence/note = Synthetic Construct
tctttttgattttacaggctcataggtggaaggaacttgccttgtctcag

SEQ ID NO: 58 - DNA, 50 bp, Artificial Sequence
  Description of Artificial Sequence/note = Synthetic Construct
agcctgatcatgtaacagaaannncaatagcgttctctggaaagaanacc

SEQ ID NO: 59 - DNA, 50 bp, Artificial Sequence
  Description of Artificial Sequence/note = Synthetic Construct
gggtgttgccaaaggaggttaacattggactcagtgggctggggagaggc

SEQ ID NO: 60 - DNA, 50 bp, Artificial Sequence
  Description of Artificial Sequence/note = Synthetic Construct
ttccagatgagattagcatttgaatcagcggactgagtaaagaagattgc

SEQ ID NO: 61 - DNA, 50 bp, Artificial Sequence
  Description of Artificial Sequence/note = Synthetic Construct
ctcaagactgcaacgtggaaatcctgctgntttwccagcctccaagcctt

SEQ ID NO: 62 - DNA, 50 bp, Artificial Sequence
  Description of Artificial Sequence/note = Synthetic Construct
ggctaggctatggtgtccagacgtttggtcaaacattagtctgggtgttt

SEQ ID NO: 63 - DNA, 50 bp, Artificial Sequence
  Description of Artificial Sequence/note = Synthetic Construct
caatgctcccagctgattaaagcctcttccttcatagaaccggtgtctaa

SEQ ID NO: 64 - DNA, 50 bp, Artificial Sequence
  Description of Artificial Sequence/note = Synthetic Construct
gcaaggagccccctgaccccttcttccaaacatactcttttgtctttgtc

SEQ ID NO: 65 - DNA, 50 bp, Artificial Sequence
  Description of Artificial Sequence/note = Synthetic Construct
atcctcctgtcccacccattggtctctcctgtcccttgattcctgcaaca

SEQ ID NO: 66 - DNA, 50 bp, Artificial Sequence
  Description of Artificial Sequence/note = Synthetic Construct
actcagaggctggtgggatcctccatatgctgaacgttggttccccgggc

SEQ ID NO: 67 - DNA, 50 bp, Artificial Sequence
  Description of Artificial Sequence/note = Synthetic Construct
aactccgtcactgtaatcccaatgtaaagcaagaattccaaaccaggaaa

SEQ ID NO: 68 - DNA, 50 bp, Artificial Sequence
  Description of Artificial Sequence/note = Synthetic Construct
gcttcattcttgaagtcagcgagaccaagaacccaccggaaggaaccaat

SEQ ID NO: 69 - DNA, 50 bp, Artificial Sequence
  Description of Artificial Sequence/note = Synthetic Construct
cttgtgtctttatttctacactctctcgtctccgcacacggggagaaaaa

SEQ ID NO: 70 - DNA, 50 bp, Artificial Sequence
  Description of Artificial Sequence/note = Synthetic Construct
aagcttcatctgtakttacagccgctccccatcactcgcattaccgcctg

SEQ ID NO: 71 - DNA, 50 bp, Artificial Sequence
  Description of Artificial Sequence/note = Synthetic Construct
tgatctgaggtggaacagtttcatcccgaaaccatccccgccccccggtc

SEQ ID NO: 72 - DNA, 50 bp, Artificial Sequence
  Description of Artificial Sequence/note = Synthetic Construct
aaaatccacggatgctcaagtccctgatataaaatggcgtagtatttgca

SEQ ID NO: 73 - DNA, 50 bp, Artificial Sequence
  Description of Artificial Sequence/note = Synthetic Construct
atgtggctaytgagcacttgaaatgtggytagtgcgactgaggaactgaa

SEQ ID NO: 74 - DNA, 50 bp, Artificial Sequence
  Description of Artificial Sequence/note = Synthetic Construct
ggacctcaagatctttaccctaaaacagttctgytgamyttcaccttggc

SEQ ID NO: 75 - DNA, 50 bp, Artificial Sequence
  Description of Artificial Sequence/note = Synthetic Construct
ttggtctccgcaaccccttatntcataacccggacattcctttccattga

SEQ ID NO: 76 - DNA, 50 bp, Artificial Sequence
  Description of Artificial Sequence/note = Synthetic Construct
cctccctctttcccctccagcccgcttttcccctttaaatattgaagccc

SEQ ID NO: 77 - DNA, 50 bp, Artificial Sequence
  Description of Artificial Sequence/note = Synthetic Construct
gtccccggaccagcagcatcagcatcacctgggaacttgttagaaatgca

SEQ ID NO: 78 - DNA, 50 bp, Artificial Sequence
  Description of Artificial Sequence/note = Synthetic Construct
tcagtattttttaaarctcyycaggtgattccaatgtgcagccaaggttg

SEQ ID NO: 79 - DNA, 50 bp, Artificial Sequence
  Description of Artificial Sequence/note = Synthetic Construct
aagtcgcagtttccaagaacctatcgacgacgttaagtgaggacttactg

SEQ ID NO: 80 - DNA, 50 bp, Artificial Sequence
  Description of Artificial Sequence/note = Synthetic Construct
aaaaatccgcgtataagtggacccacgcagttcaaacccgtgttgttcaa

SEQ ID NO: 81 - DNA, 50 bp, Artificial Sequence
  Description of Artificial Sequence/note = Synthetic Construct
gctgtgagacccctgatttcccacttcacacctctatatttctgtgtgtg

SEQ ID NO: 82 - DNA, 50 bp, Artificial Sequence
  Description of Artificial Sequence/note = Synthetic Construct
tgattttgcccttgtcctgtttcctcagaagcatgtgatctttgttctcc

SEQ ID NO: 83 - DNA, 50 bp, Artificial Sequence
  Description of Artificial Sequence/note = Synthetic Construct
acttgctggtttttgcggcttgtggggcatcacggaacctaccgacatgt

SEQ ID NO: 84 - DNA, 50 bp, Artificial Sequence
  Description of Artificial Sequence/note = Synthetic Construct
ccccacaacaaagaattatccggcccaaaatgtcgatagtgccaaggttg

SEQ ID NO: 85 - DNA, 50 bp, Artificial Sequence
  Description of Artificial Sequence/note = Synthetic Construct
sagcagaggraaaacatggtttgagagaggttttyctgmaayagragggc

SEQ ID NO: 86 - DNA, 50 bp, Artificial Sequence
  Description of Artificial Sequence/note = Synthetic Construct
cggtcagaagcacaggtnacaacctggngcttgcgactggcatctgaagt

SEQ ID NO: 87 - DNA, 50 bp, Artificial Sequence
  Description of Artificial Sequence/note = Synthetic Construct
tgagtctccccaaaagtggagcccttgtgatgacgagcacaggtccgcct

SEQ ID NO: 88 - DNA, 50 bp, Artificial Sequence
  Description of Artificial Sequence/note = Synthetic Construct
aagacgangaggatgaagacctttatgatgatccacttccacttaatgaa

SEQ ID NO: 89 - DNA, 50 bp, Artificial Sequence
  Description of Artificial Sequence/note = Synthetic Construct
ttttaagaaagtttacgaatttgtgttgggccgcattcaaagccatcctg

SEQ ID NO: 90 - DNA, 50 bp, Artificial Sequence
  Description of Artificial Sequence/note = Synthetic Construct
gatgaaaaggggatcctgtgcagaaaccacactacccatcagagaagcaa

SEQ ID NO: 91 - DNA, 50 bp, Artificial Sequence
  Description of Artificial Sequence/note = Synthetic Construct
ggcaggtcatagaaactagaactcctctcccccaaagcaagccataaaac

SEQ ID NO: 92 - DNA, 50 bp, Artificial Sequence
  Description of Artificial Sequence/note = Synthetic Construct
agggttcggtactatccgcggtttcaggcatccactgggggtcttggaac

SEQ ID NO: 93 - DNA, 50 bp, Artificial Sequence
  Description of Artificial Sequence/note = Synthetic Construct
cgcacctcaaactgcaaaagttacggccacagtgcgtgataagtgcttag

SEQ ID NO: 94 - DNA, 50 bp, Artificial Sequence
  Description of Artificial Sequence/note = Synthetic Construct
gaaattcttaataatttttgaacaaggggccccgcattttcattttgcac

SEQ ID NO: 95 - DNA, 50 bp, Artificial Sequence
  Description of Artificial Sequence/note = Synthetic Construct
tgttgttgtggacgcgctctcggggttsgaaccgayacaagarccttaca

SEQ ID NO: 96 - DNA, 50 bp, Artificial Sequence
  Description of Artificial Sequence/note = Synthetic Construct
tcttccttggcaatamtyrttgtctcagtgattggctttctgtgcagtga

SEQ ID NO: 97 - DNA, 50 bp, Artificial Sequence
  Description of Artificial Sequence/note = Synthetic Construct
ggggaaaggtggggaaaagattgagaaatcggatggttgccgtgtctgtg

SEQ ID NO: 98 - DNA, 50 bp, Artificial Sequence
  Description of Artificial Sequence/note = Synthetic Construct
gtggagatttcagccgctttgaggtcaatggtagaataggaaatatcttc

SEQ ID NO: 99 - DNA, 50 bp, Artificial Sequence
  Description of Artificial Sequence/note = Synthetic Construct
ggagtcaagaccccccagcccctcctccctcagactcatgagtccagacc

SEQ ID NO: 100 - DNA, 50 bp, Artificial Sequence
  Description of Artificial Sequence/note = Synthetic Construct
actcatggagggttagggttcaggttcgggttcgggttcgggttcgggtt

SEQ ID NO: 101 - DNA, 50 bp, Artificial Sequence
  Description of Artificial Sequence/note = Synthetic Construct
ggttctgagtgtttgtccctcacataggattccagaacactgctgctggg

SEQ ID NO: 102 - DNA, 50 bp, Artificial Sequence
  Description of Artificial Sequence/note = Synthetic Construct
tcacaatgcccctgtaggcagagcctagacaagagttacatcacctgggt

SEQ ID NO: 103 - DNA, 50 bp, Artificial Sequence
  Description of Artificial Sequence/note = Synthetic Construct
gggtccattcgatgatgatcacactggatttcattccataattctattcg

SEQ ID NO: 104 - DNA, 50 bp, Artificial Sequence
  Description of Artificial Sequence/note = Synthetic Construct
ccactgtctgtgctgtgtctttcaaaggtcagaagagattgnacctttgt

SEQ ID NO: 105 - DNA, 50 bp, Artificial Sequence
  Description of Artificial Sequence/note = Synthetic Construct
tgcrtttacaaacctttagctagacacagagcgctgattggtgcgttttt

SEQ ID NO: 106 - DNA, 50 bp, Artificial Sequence
  Description of Artificial Sequence/note = Synthetic Construct
cctgactcctgagtcacgttactgtcccactatacgttaagaggagggaa

SEQ ID NO: 107 - DNA, 50 bp, Artificial Sequence
  Description of Artificial Sequence/note = Synthetic Construct
aatatcaggaacaccggcatgtgcacttaggaccatgttttaatttttca

SEQ ID NO: 108 - DNA, 50 bp, Artificial Sequence
  Description of Artificial Sequence/note = Synthetic Construct
ggaatggaatggaatggaatggaatggaatggaatggaatggaatggaat

SEQ ID NO: 109 - DNA, 50 bp, Artificial Sequence
  Description of Artificial Sequence/note = Synthetic Construct
ggatgaggcaggaaagacagctgagggtcagaacccaggcaggtccaatg

SEQ ID NO: 110 - DNA, 50 bp, Artificial Sequence
  Description of Artificial Sequence/note = Synthetic Construct
actcgctgaaggctcagatgatcgttagcattttttagcaataaagtatt

SEQ ID NO: 111 - DNA, 50 bp, Artificial Sequence
  Description of Artificial Sequence/note = Synthetic Construct
taaagttacaccgagtgtgcctgcctctcctgcctccccttccacctcct

SEQ ID NO: 112 - DNA, 50 bp, Artificial Sequence
  Description of Artificial Sequence/note = Synthetic Construct
gggactcaggaggatgttgagggagacagaggggtgaagcgttgagacga

SEQ ID NO: 113 - DNA, 50 bp, Artificial Sequence
  Description of Artificial Sequence/note = Synthetic Construct
caggcggccagnctttcagggggaggatgaagtaggcctgggacaaaagc

SEQ ID NO: 114 - DNA, 50 bp, Artificial Sequence
  Description of Artificial Sequence/note = Synthetic Construct
aggactctacttctaatagtatggagaacactgatagtccttggcatgaa

SEQ ID NO: 115 - DNA, 50 bp, Artificial Sequence
  Description of Artificial Sequence/note = Synthetic Construct
ccctgtcacttgggttaagaccattggaagtacatcgattataaatctca

SEQ ID NO: 116 - DNA, 50 bp, Artificial Sequence
  Description of Artificial Sequence/note = Synthetic Construct
aacccaacagtatcaggtgctcagaaccgatgaagaagctcaagattgag

SEQ ID NO: 117 - DNA, 50 bp, Artificial Sequence
  Description of Artificial Sequence/note = Synthetic Construct
tggttaatgtgtaacaaggaggcagtaggccccaggtgtccagccagagg

SEQ ID NO: 118 - DNA, 50 bp, Artificial Sequence
  Description of Artificial Sequence/note = Synthetic Construct
aaaagtgaggacgagagtaagaactcccactaaaagtgaaaattctcaaa

SEQ ID NO: 119 - DNA, 50 bp, Artificial Sequence
  Description of Artificial Sequence/note = Synthetic Construct
cataccaccccccaaaaattttcactgccccaacacttcaacactatttt

SEQ ID NO: 120 - DNA, 50 bp, Artificial Sequence
  Description of Artificial Sequence/note = Synthetic Construct
ttgtaggatgctgtgtcataccctgtgccctaggattaatacaaaagctc

SEQ ID NO: 121 - DNA, 50 bp, Artificial Sequence
  Description of Artificial Sequence/note = Synthetic Construct
gcctccactctttatgaactcttaacctgtctcttctcattcctttgtca

SEQ ID NO: 122 - DNA, 50 bp, Artificial Sequence
  Description of Artificial Sequence/note = Synthetic Construct
ccggatcattcacagagttcaattcaattaacagtttaagcccccaaaaa

SEQ ID NO: 123 - DNA, 50 bp, Artificial Sequence
  Description of Artificial Sequence/note = Synthetic Construct
agagatcagacgaaacctgagaccagagactcattttcttctaaaatgct

SEQ ID NO: 124 - DNA, 50 bp, Artificial Sequence
  Description of Artificial Sequence/note = Synthetic Construct
acatgcatgtttgttcaatacgcatgcgtcaggaccaccttcatgaatat

SEQ ID NO: 125 - DNA, 50 bp, Artificial Sequence
  Description of Artificial Sequence/note = Synthetic Construct
caaccccccttatcttaactcaagctgacttcaactcttcaggcagagct

SEQ ID NO: 126 - DNA, 50 bp, Artificial Sequence
  Description of Artificial Sequence/note = Synthetic Construct
gccctcctgtctctcagtcccattctcccccgaggctagccatagaaact

SEQ ID NO: 127 - DNA, 50 bp, Artificial Sequence
  Description of Artificial Sequence/note = Synthetic Construct
tcttggagaagggatccttgttccccnctggcnctggtannccactgcag

SEQ ID NO: 128 - DNA, 50 bp, Artificial Sequence
  Description of Artificial Sequence/note = Synthetic Construct
aagcctaawttttcgtggccgtgtgacaaggaccccgtctttagctgaac

SEQ ID NO: 129 - DNA, 50 bp, Artificial Sequence
  Description of Artificial Sequence/note = Synthetic Construct
caacccttgccaaatgaagagaactgccttcncatgaagaattaantagt

SEQ ID NO: 130 - DNA, 50 bp, Artificial Sequence
  Description of Artificial Sequence/note = Synthetic Construct
gcacagagccatacaactaatacccctacttatagggttaggaatggcta

SEQ ID NO: 131 - DNA, 50 bp, Artificial Sequence
  Description of Artificial Sequence/note = Synthetic Construct
aaactggactaatgtccttgtcccaacaggtagatgctgatttaaataac

SEQ ID NO: 132 - DNA, 50 bp, Artificial Sequence
  Description of Artificial Sequence/note = Synthetic Construct
cacttcttcaagcatctcgacaactttttgcagggaaaacgcttccacaa

SEQ ID NO: 133 - DNA, 50 bp, Artificial Sequence
  Description of Artificial Sequence/note = Synthetic Construct
tggtatcatcgcttacaaaagtgtcttgaacttgatggagcttatgttga

SEQ ID NO: 134 - DNA, 50 bp, Artificial Sequence
  Description of Artificial Sequence/note = Synthetic Construct
aaacaaccccatcaaaaagtgggcaaaggatatgaacagacacttctcaa

SEQ ID NO: 135 - DNA, 50 bp, Artificial Sequence
  Description of Artificial Sequence/note = Synthetic Construct
gtgatggtttcacgggtgtatgcatatgtccaaactcatcaaattgtata

SEQ ID NO: 136 - DNA, 50 bp, Artificial Sequence
  Description of Artificial Sequence/note = Synthetic Construct
tcagtttgggaagatgaaaaagttctggagatggatggtggtgatggttg

SEQ ID NO: 137 - DNA, 50 bp, Artificial Sequence
  Description of Artificial Sequence/note = Synthetic Construct
agatagtggtgatggttgcacaactctgtgaatatactaaaaaccactga

SEQ ID NO: 138 - DNA, 50 bp, Artificial Sequence
  Description of Artificial Sequence/note = Synthetic Construct
atgttaataataggggaaactgtgtgngggnggggtgagggggtatatgg

SEQ ID NO: 139 - DNA, 50 bp, Artificial Sequence
  Description of Artificial Sequence/note = Synthetic Construct
ctgttggagtgggaggttacagataagcaaggggaggaggctagaatgat

SEQ ID NO: 140 - DNA, 50 bp, Artificial Sequence
  Description of Artificial Sequence/note = Synthetic Construct
tatttaggggtaanggggcatcatgtctgcaacttactctcaaatggttc

SEQ ID NO: 141 - DNA, 50 bp, Artificial Sequence
  Description of Artificial Sequence/note = Synthetic Construct
gcaggagggaagtgggtgtggctataaaagggcaacatgagggatccttg

SEQ ID NO: 142 - DNA, 50 bp, Artificial Sequence
  Description of Artificial Sequence/note = Synthetic Construct
gngngggggaagggaggtgggtgtggctataaaagggcagcacgagggat

SEQ ID NO: 143 - DNA, 50 bp, Artificial Sequence
  Description of Artificial Sequence/note = Synthetic Construct
agtggttgcctctggggagggtgantgactggaaaggggcatgagggaac

SEQ ID NO: 144 - DNA, 50 bp, Artificial Sequence
  Description of Artificial Sequence/note = Synthetic Construct
ggcaaaactaatctatgstgttagaagtcaggatagtggttacccttggg

SEQ ID NO: 145 - DNA, 50 bp, Artificial Sequence
  Description of Artificial Sequence/note = Synthetic Construct
ggtgttgggagagcctcagccggaatttcgtggacggacaagggcacaga

SEQ ID NO: 146 - DNA, 50 bp, Artificial Sequence
  Description of Artificial Sequence/note = Synthetic Construct
ctagaggtttgagcagcggggcactgaagaagcgagccacacccccatcg

SEQ ID NO: 147 - DNA, 50 bp, Artificial Sequence
  Description of Artificial Sequence/note = Synthetic Construct
atcctcctcaaccccatcggtctctctgattcctaaatcatccccaaaca

SEQ ID NO: 148 - DNA, 50 bp, Artificial Sequence
  Description of Artificial Sequence/note = Synthetic Construct
tttctctattgcaattcccctgtcttgatgaatcggctctgtctaggcag

SEQ ID NO: 149 - DNA, 50 bp, Artificial Sequence
  Description of Artificial Sequence/note = Synthetic Construct
taaactcctcgtgtgtgtccgtgtcctaaattttcctggcgcgngacgac

SEQ ID NO: 150 - DNA, 50 bp, Artificial Sequence
  Description of Artificial Sequence/note = Synthetic Construct
cctgtacctatcgcaatggtcctgaataaagtctgccttaccgtgcttta

SEQ ID NO: 151 - DNA, 50 bp, Artificial Sequence
  Description of Artificial Sequence/note = Synthetic Construct
gcccaaacccctttgtcttgtcacgttttcacaatttactactctttgtc

SEQ ID NO: 152 - DNA, 50 bp, Artificial Sequence
  Description of Artificial Sequence/note = Synthetic Construct
gcaacgtcaggaagttaccctatatggtctaaaaaggggaggcatgaata

SEQ ID NO: 153 - DNA, 50 bp, Artificial Sequence
  Description of Artificial Sequence/note = Synthetic Construct
tgccatggcaacgtcaggaagttaccctatatggtctaaaaaggggagga

SEQ ID NO: 154 - DNA, 50 bp, Artificial Sequence
  Description of Artificial Sequence/note = Synthetic Construct
tagcagagcacatctcccccgtaatgttctttggctttgttatcctatat

SEQ ID NO: 155 - DNA, 50 bp, Artificial Sequence
  Description of Artificial Sequence/note = Synthetic Construct
tggccctcttccaagtgtacttcgcttcctttcgttcctgctctaaaact

SEQ ID NO: 156 - DNA, 50 bp, Artificial Sequence
  Description of Artificial Sequence/note = Synthetic Construct
ttcaagctaccaacgtgatgtcactgaatgsggagttgggaaaagatata

SEQ ID NO: 157 - DNA, 50 bp, Artificial Sequence
  Description of Artificial Sequence/note = Synthetic Construct
atgtcactgaatgsggagttgggaagagatgcacagtagcacacyattat

SEQ ID NO: 158 - DNA, 50 bp, Artificial Sequence
  Description of Artificial Sequence/note = Synthetic Construct
acaatgtaacggctacagacacgacacacttttaagtttaatctgcatta

SEQ ID NO: 159 - DNA, 50 bp, Artificial Sequence
  Description of Artificial Sequence/note = Synthetic Construct
gaatatgcacatagtttactatggcacgcgtattcccattgcaatgctct

SEQ ID NO: 160 - DNA, 50 bp, Artificial Sequence
  Description of Artificial Sequence/note = Synthetic Construct
acatttgcctgacaactgtctcacraacctagctactgcaagagcctact

SEQ ID NO: 161 - DNA, 50 bp, Artificial Sequence
  Description of Artificial Sequence/note = Synthetic Construct
agactagctgaaacagggccagggcaaaagcacctctccataagacacac

SEQ ID NO: 162 - DNA, 50 bp, Artificial Sequence
  Description of Artificial Sequence/note = Synthetic Construct
cttgaacaccagaccaaattgaagactagctgaaacagggccagggcaaa

SEQ ID NO: 163 - DNA, 50 bp, Artificial Sequence
  Description of Artificial Sequence/note = Synthetic Construct
gcctcaacctcggcctataaagacttgaacaaacactaacatagtttcta

SEQ ID NO: 164 - DNA, 50 bp, Artificial Sequence
  Description of Artificial Sequence/note = Synthetic Construct
cacagaacaactccatccaaacccctgcactaagagacttgaccaaactc

SEQ ID NO: 165 - DNA, 50 bp, Artificial Sequence
  Description of Artificial Sequence/note = Synthetic Construct
tcttgagaacatgtatgtaatgggctgtatctgctcggctatataaaagg

SEQ ID NO: 166 - DNA, 50 bp, Artificial Sequence
  Description of Artificial Sequence/note = Synthetic Construct
aaccctgggcactgagtctctaatgagcttccctggtagacaacatttca

SEQ ID NO: 167 - DNA, 50 bp, Artificial Sequence
  Description of Artificial Sequence/note = Synthetic Construct
ttccctttgctgatcttgccgtgtatccttacnrtgtcgctgtaataaat

SEQ ID NO: 168 - DNA, 50 bp, Artificial Sequence
  Description of Artificial Sequence/note = Synthetic Construct
cccccaaattgtataagcttcaggccccacaaaacctggatctgcccctg

SEQ ID NO: 169 - DNA, 50 bp, Artificial Sequence
  Description of Artificial Sequence/note = Synthetic Construct
ttacaaaatcattgtcatatgaagaggcgatcaaagagtatgcagccaaa

SEQ ID NO: 170 - DNA, 50 bp, Artificial Sequence
  Description of Artificial Sequence/note = Synthetic Construct
tgttctgtctcaccggactcagacaagttggtaaccagtgcacagtgaac

SEQ ID NO: 171 - DNA, 50 bp, Artificial Sequence
  Description of Artificial Sequence/note = Synthetic Construct
tcngacccctattcctggtggttggcatagtgatgatctttgctattctc

SEQ ID NO: 172 - DNA, 50 bp, Artificial Sequence
  Description of Artificial Sequence/note = Synthetic Construct
ggcatgaagctcaattgcacatgtgcatgtttctcctttcataaatattc

SEQ ID NO: 173 - DNA, 50 bp, Artificial Sequence
  Description of Artificial Sequence/note = Synthetic Construct
ggtgacggggtacgactgggtttcaaacaacttatgtcaggcctaaaaat

SEQ ID NO: 174 - DNA, 50 bp, Artificial Sequence
  Description of Artificial Sequence/note = Synthetic Construct
gggggtatgggctctggattggttggtttgcatatgaaaggcgcgctccc

SEQ ID NO: 175 - DNA, 50 bp, Artificial Sequence
  Description of Artificial Sequence/note = Synthetic Construct
tggccgaagattcatttgatgaatccgatttttccgaaatagacgattct

SEQ ID NO: 176 - DNA, 50 bp, Artificial Sequence
  Description of Artificial Sequence/note = Synthetic Construct
tgttgccttaatcggctnctctgacacccggcagctcagctctctctcca

SEQ ID NO: 177 - DNA, 50 bp, Artificial Sequence
  Description of Artificial Sequence/note = Synthetic Construct
ggtgagcttccctggttggcaatactctntgcatgttgtcacacatcgtt

SEQ ID NO: 178 - DNA, 50 bp, Artificial Sequence
  Description of Artificial Sequence/note = Synthetic Construct
ccataggcttcaccagactgccaaaggggcccatggcacaaaaaaggtta

SEQ ID NO: 179 - DNA, 50 bp, Artificial Sequence
  Description of Artificial Sequence/note = Synthetic Construct
ntgcaaatgaccgngaaagtgctncaagtattgattttggggttacaaat

SEQ ID NO: 180 - DNA, 50 bp, Artificial Sequence
  Description of Artificial Sequence/note = Synthetic Construct
cacaaattctttgacactcttcccatcgaggagtggggtccgtntcctct

SEQ ID NO: 181 - DNA, 50 bp, Artificial Sequence
  Description of Artificial Sequence/note = Synthetic Construct
aataaaaactctcttcctccccagttcatctgcatctcgttattgggcca

SEQ ID NO: 182 - DNA, 50 bp, Artificial Sequence
  Description of Artificial Sequence/note = Synthetic Construct
ccagttcatctgcatctcgttattgggccacgagaataagcagcccgacc

SEQ ID NO: 183 - DNA, 50 bp, Artificial Sequence
  Description of Artificial Sequence/note = Synthetic Construct
gcagttatgggggatactcggctctttgcacatttggatnagagaagcat

SEQ ID NO: 184 - DNA, 50 bp, Artificial Sequence
  Description of Artificial Sequence/note = Synthetic Construct
cctggataaattcccctggggaacttgaggccccatatacacgaaattac

SEQ ID NO: 185 - DNA, 50 bp, Artificial Sequence
  Description of Artificial Sequence/note = Synthetic Construct
tttgttgggaactcagttacaaataaccctcaccataccagtactttctg

SEQ ID NO: 186 - DNA, 50 bp, Artificial Sequence
  Description of Artificial Sequence/note = Synthetic Construct
catgcttaaggagcccttcagcctgccactgcactgtgggaacactggcc

SEQ ID NO: 187 - DNA, 50 bp, Artificial Sequence
  Description of Artificial Sequence/note = Synthetic Construct
cgcctcctccacaaagaagaaccaaaatagcgagtagataatcacacttt

SEQ ID NO: 188 - DNA, 50 bp, Artificial Sequence
  Description of Artificial Sequence/note = Synthetic Construct
tgctccatctgcgagacgcacccttctatagaagtaaaattgccttgctg

SEQ ID NO: 189 - DNA, 50 bp, Artificial Sequence
  Description of Artificial Sequence/note = Synthetic Construct
gctgagagaccctttgtcctttggctcagtgttggttcttctttgcagca

SEQ ID NO: 190 - DNA, 50 bp, Artificial Sequence
  Description of Artificial Sequence/note = Synthetic Construct
cagtgtactctcatggcaaaactgctggtgagtgtaccctttctgcagaa

SEQ ID NO: 191 - DNA, 50 bp, Artificial Sequence
  Description of Artificial Sequence/note = Synthetic Construct
ctgcattgcagcccaacttctccctctgcccaatcctgcttccttccctt

SEQ ID NO: 192 - DNA, 50 bp, Artificial Sequence
  Description of Artificial Sequence/note = Synthetic Construct
ccaagaaccccaggtcagagaacacgaggcttgccaccatcttggaagtg

SEQ ID NO: 193 - DNA, 50 bp, Artificial Sequence
  Description of Artificial Sequence/note = Synthetic Construct
gcacgtaggcacagcttagtttagtctttacatagacaagactcctatat

SEQ ID NO: 194 - DNA, 50 bp, Artificial Sequence
  Description of Artificial Sequence/note = Synthetic Construct
tccgcaaccaatcagacgtttgcataggagtgtaactttgtaacttcact

SEQ ID NO: 195 - DNA, 50 bp, Artificial Sequence
  Description of Artificial Sequence/note = Synthetic Construct
ctttacttcgtcctcttcatttacatagggcgtaccccaagtaaccaatg

SEQ ID NO: 196 - DNA, 50 bp, Artificial Sequence
  Description of Artificial Sequence/note = Synthetic Construct
atcttctaccacatggctgcactggagtctctgaacctactctggttctg

SEQ ID NO: 197 - DNA, 50 bp, Artificial Sequence
  Description of Artificial Sequence/note = Synthetic Construct
tataaatttgttccgaccacgaggcatccctggagtctctctgaatctgc

SEQ ID NO: 198 - DNA, 50 bp, Artificial Sequence
  Description of Artificial Sequence/note = Synthetic Construct
caaccctggctgctgaaactgcctgttgtaacctgaaaccagttttatct

SEQ ID NO: 199 - DNA, 50 bp, Artificial Sequence
  Description of Artificial Sequence/note = Synthetic Construct
tctgcagcccaagaaccatcctataaaatctccagcaagcctttgtctcc

SEQ ID NO: 200 - DNA, 50 bp, Artificial Sequence
  Description of Artificial Sequence/note = Synthetic Construct
cataaatgctcctaaggaaaaatccaccgcggcgcgctcagtcctctctt

SEQ ID NO: 201 - DNA, 50 bp, Artificial Sequence
  Description of Artificial Sequence/note = Synthetic Construct
ttgactatgatgtgtaggaggggtagggctgctttagtaaaatgagtaag

SEQ ID NO: 202 - DNA, 50 bp, Artificial Sequence
  Description of Artificial Sequence/note = Synthetic Construct
gaaggcacccctcccgaggaaatctcaactgcacgacccctactacgccc

SEQ ID NO: 203 - DNA, 50 bp, Artificial Sequence
  Description of Artificial Sequence/note = Synthetic Construct
gttctcaaccttcctaatgccgcggccctttaatacagttcctgtgggtc

SEQ ID NO: 204 - DNA, 50 bp, Artificial Sequence
  Description of Artificial Sequence/note = Synthetic Construct
tgaaagatacactgtaaacacccacaaccamcttccctggagccccatca

SEQ ID NO: 205 - DNA, 50 bp, Artificial Sequence
  Description of Artificial Sequence/note = Synthetic Construct
tgtacatacggcttgcgcccaggctcactcgcgcccagagagagagtaaa

SEQ ID NO: 206 - DNA, 50 bp, Artificial Sequence
  Description of Artificial Sequence/note = Synthetic Construct
atgagagagctgctgaataaaaccatatttcacctgcctacggccccccg

SEQ ID NO: 207 - DNA, 50 bp, Artificial Sequence
  Description of Artificial Sequence/note = Synthetic Construct
agagagtgctcctgactgaaatcggccagaagcccctctcaggtttattc

SEQ ID NO: 208 - DNA, 50 bp, Artificial Sequence
  Description of Artificial Sequence/note = Synthetic Construct
gactgkwgagccgcttttcgtgtttctttcctctttctttaattcttaca

SEQ ID NO: 209 - DNA, 50 bp, Artificial Sequence
  Description of Artificial Sequence/note = Synthetic Construct
aataaattctgctcyacctcacccttcaatgtgtctgcatgcctaattct

SEQ ID NO: 210 - DNA, 50 bp, Artificial Sequence
  Description of Artificial Sequence/note = Synthetic Construct
gtaactngcttgataacgcaccctttattggcttccttcccttccctgtc

SEQ ID NO: 211 - DNA, 50 bp, Artificial Sequence
  Description of Artificial Sequence/note = Synthetic Construct
ctgcttyccttgactgtkawgggggcagccgrcaggttaataaargcttg

SEQ ID NO: 212 - DNA, 50 bp, Artificial Sequence
  Description of Artificial Sequence/note = Synthetic Construct
caataaagcttgcttgcctgactttgggtctcytcatcctttctctcggc

SEQ ID NO: 213 - DNA, 50 bp, Artificial Sequence
  Description of Artificial Sequence/note = Synthetic Construct
ttgagcagtaggatataaataactcccacatgcttagcgttccaataatg

SEQ ID NO: 214 - DNA, 50 bp, Artificial Sequence
  Description of Artificial Sequence/note = Synthetic Construct
gtgcyagctgnttagggccagcwgcwgtkacaaacctyycttggwgtstg

SEQ ID NO: 215 - DNA, 50 bp, Artificial Sequence
  Description of Artificial Sequence/note = Synthetic Construct
cctttaaaaaccacttgtaactgctgctaattggagtgtatattcagggc

SEQ ID NO: 216 - DNA, 50 bp, Artificial Sequence
  Description of Artificial Sequence/note = Synthetic Construct
aaaccttaacttctccactttggaacgctgaccccattcctttggagtct

SEQ ID NO: 217 - DNA, 50 bp, Artificial Sequence
  Description of Artificial Sequence/note = Synthetic Construct
gtcctgtccccccaaccatgtgagatagagccatctgggaatgagcttta

SEQ ID NO: 218 - DNA, 50 bp, Artificial Sequence
  Description of Artificial Sequence/note = Synthetic Construct
agcgggaatattagtggtgagttgttgctccctgtattgttgctgtggcc

SEQ ID NO: 219 - DNA, 50 bp, Artificial Sequence
  Description of Artificial Sequence/note = Synthetic Construct
acttactggctgtcgwgcggtgagcagtaccagctttggattcagttaca

SEQ ID NO: 220 - DNA, 50 bp, Artificial Sequence
  Description of Artificial Sequence/note = Synthetic Construct
aatggcagtcgtctcctgatctgttggccttaccatacctgaataataat

SEQ ID NO: 221 - DNA, 50 bp, Artificial Sequence
  Description of Artificial Sequence/note = Synthetic Construct
cttttcaatggcagtcgtctcctgatctgttggccttaccatacctsaat

SEQ ID NO: 222 - DNA, 50 bp, Artificial Sequence
  Description of Artificial Sequence/note = Synthetic Construct
aggggaacttgtggcagggaccagccttatcacactggtgcacctggtca

SEQ ID NO: 223 - DNA, 50 bp, Artificial Sequence
  Description of Artificial Sequence/note = Synthetic Construct
gagcccagtctgctaggcgggagagatgcctctaagttcttatctctggc

SEQ ID NO: 224 - DNA, 50 bp, Artificial Sequence
  Description of Artificial Sequence/note = Synthetic Construct
ggctcctgaaccttctcctaggcccatctgtgcacttccttgtaaaatcc

SEQ ID NO: 225 - DNA, 50 bp, Artificial Sequence
  Description of Artificial Sequence/note = Synthetic Construct
gccctgtccttggcctgcwtagcccagttttagcaagaatcctgctaagt

SEQ ID NO: 226 - DNA, 50 bp, Artificial Sequence
  Description of Artificial Sequence/note = Synthetic Construct
atccacctgccttttgtttcagnggagttgagttcaanctctaaccccta

SEQ ID NO: 227 - DNA, 50 bp, Artificial Sequence
  Description of Artificial Sequence/note = Synthetic Construct
gatgattcagctggtccttaatgaacaaaaggcmacccaacaagaaaatg

SEQ ID NO: 228 - DNA, 50 bp, Artificial Sequence
  Description of Artificial Sequence/note = Synthetic Construct
ttccacattgcaactaacctttaagaaactaccacttgtcgagttttggt

SEQ ID NO: 229 - DNA, 50 bp, Artificial Sequence
  Description of Artificial Sequence/note = Synthetic Construct
caccgcaactaacctttaagaaactaccacttgttgagttttggtgtagt

SEQ ID NO: 230 - DNA, 50 bp, Artificial Sequence
  Description of Artificial Sequence/note = Synthetic Construct
cagtggagttttccagaggctacatgacgtgtgatgtcgcaacagattga

SEQ ID NO: 231 - DNA, 50 bp, Artificial Sequence
  Description of Artificial Sequence/note = Synthetic Construct
taaaattctgtgggggaagtggaatggaaatacgagttcaaggagaaaaa

SEQ ID NO: 232 - DNA, 50 bp, Artificial Sequence
  Description of Artificial Sequence/note = Synthetic Construct
caatcttttggcttccctgggccacattggaagaagaattgtcttgggcc

SEQ ID NO: 233 - DNA, 50 bp, Artificial Sequence
  Description of Artificial Sequence/note = Synthetic Construct
ccgcatacgagttaaatgctcttatatttgcatttaaaactggcattgca

SEQ ID NO: 234 - DNA, 50 bp, Artificial Sequence
  Description of Artificial Sequence/note = Synthetic Construct
gcgagtatccccgtgcccgagggagcgtgacattaaatagcaaataaaaa

SEQ ID NO: 235 - DNA, 50 bp, Artificial Sequence
  Description of Artificial Sequence/note = Synthetic Construct
ctctccgctgrcagagagctttcttctttcacttattaaactttcactcc

SEQ ID NO: 236 - DNA, 50 bp, Artificial Sequence
  Description of Artificial Sequence/note = Synthetic Construct
tctcagtgtaattggtctgttactgcgcagtgggcatatgaacctgttgg

SEQ ID NO: 237 - DNA, 50 bp, Artificial Sequence
  Description of Artificial Sequence/note = Synthetic Construct
atcccgactcctgcgagaagtagctcaccgtgacaaagctgcctttgctt

SEQ ID NO: 238 - DNA, 50 bp, Artificial Sequence
  Description of Artificial Sequence/note = Synthetic Construct
tctctcaagaataccccaaaaattaagtttttctttttccaaggtgccca

SEQ ID NO: 239 - DNA, 50 bp, Artificial Sequence
  Description of Artificial Sequence/note = Synthetic Construct
cctgtgatctcgccctgcctccacttgccttgtgatattctattaccytg

SEQ ID NO: 240 - DNA, 50 bp, Artificial Sequence
  Description of Artificial Sequence/note = Synthetic Construct
ttcatccccatgtgaccatctcacctcataatcaaatgaccctaaatccc

SEQ ID NO: 241 - DNA, 50 bp, Artificial Sequence
  Description of Artificial Sequence/note = Synthetic Construct
ggcgactggccaaggagaagcacccctctgcgcagaagtaaaattgcttt

SEQ ID NO: 242 - DNA, 50 bp, Artificial Sequence
  Description of Artificial Sequence/note = Synthetic Construct
ccacactcgcgatggccccctggtcccactttctctctcaaactgtcttt

SEQ ID NO: 243 - DNA, 50 bp, Artificial Sequence
  Description of Artificial Sequence/note = Synthetic Construct
tttgcagcctccatacttagcgttggccccctggacccactttctctctc

SEQ ID NO: 244 - DNA, 50 bp, Artificial Sequence
  Description of Artificial Sequence/note = Synthetic Construct
gtgggacaagaacttgggaatcagtgcacaagccagacttggcctgggaa

SEQ ID NO: 245 - DNA, 50 bp, Artificial Sequence
  Description of Artificial Sequence/note = Synthetic Construct
attgatccccacccttcacctattttacatatacccaccctttcctaatt

SEQ ID NO: 246 - DNA, 50 bp, Artificial Sequence
  Description of Artificial Sequence/note = Synthetic Construct
ttaatcaatctgccttntgtcagtgatttttcagcgaaccttcagggggc

SEQ ID NO: 247 - DNA, 50 bp, Artificial Sequence
  Description of Artificial Sequence/note = Synthetic Construct
ctttttttctctcttggtccgatccgtgtctctcwctcgccgcgggcwgc

SEQ ID NO: 248 - DNA, 50 bp, Artificial Sequence
  Description of Artificial Sequence/note = Synthetic Construct
tttctcttttgcaaaacccatcgtcacagtgattgrcttactgcgcgcgg

SEQ ID NO: 249 - DNA, 50 bp, Artificial Sequence
  Description of Artificial Sequence/note = Synthetic Construct
accctttcctgactgattctctctgaataatgcccacctgcgcactggga

SEQ ID NO: 250 - DNA, 50 bp, Artificial Sequence
  Description of Artificial Sequence/note = Synthetic Construct
ccgacccgccccacaagtgtttacatcagatgcttttgtgcagatgaggg

SEQ ID NO: 251 - DNA, 50 bp, Artificial Sequence
  Description of Artificial Sequence/note = Synthetic Construct
cgcttgcccactgtcycctttctactggttctgcttaycyctccctataa

SEQ ID NO: 252 - DNA, 50 bp, Artificial Sequence
  Description of Artificial Sequence/note = Synthetic Construct
ttctgcctgaactttgagatgcttgcagatcttatggtcagagcgttctc

SEQ ID NO: 253 - DNA, 50 bp, Artificial Sequence
  Description of Artificial Sequence/note = Synthetic Construct
tatctaccccttcctataaaagtccaaggcaaaaccaccctgccgagaca

SEQ ID NO: 254 - DNA, 50 bp, Artificial Sequence
  Description of Artificial Sequence/note = Synthetic Construct
gccctgggttcctacgtaagcaaaccgaaacctaactcagncgtttctta

SEQ ID NO: 255 - DNA, 50 bp, Artificial Sequence
  Description of Artificial Sequence/note = Synthetic Construct
cacagatgcatgagggagcccagccgagaccagaagaaccacccagctga

SEQ ID NO: 256 - DNA, 50 bp, Artificial Sequence
  Description of Artificial Sequence/note = Synthetic Construct
gaacccagaaacaaatccatacatytacagcgaactcattttcgacaaag

SEQ ID NO: 257 - DNA, 50 bp, Artificial Sequence
  Description of Artificial Sequence/note = Synthetic Construct
atgtaagtccccaataaaccctatgtctcatttgctggctctgggtctct

SEQ ID NO: 258 - DNA, 50 bp, Artificial Sequence
  Description of Artificial Sequence/note = Synthetic Construct
gcacaacgacgaaatcgcctaacgacgcatttctcagaacgtatccccgt

SEQ ID NO: 259 - DNA, 50 bp, Artificial Sequence
  Description of Artificial Sequence/note = Synthetic Construct
tagtgacacctttgctttctgatggttcaatgtacacaaactttgtttca

SEQ ID NO: 260 - DNA, 50 bp, Artificial Sequence
  Description of Artificial Sequence/note = Synthetic Construct
cggattttcagatttgggatgctcaaccggtaagtataatgcaaatattc

SEQ ID NO: 261 - DNA, 50 bp, Artificial Sequence
  Description of Artificial Sequence/note = Synthetic Construct
nctgccagncaacnacagnttgtgcacctngntggcaraganactgacac

SEQ ID NO: 262 - DNA, 50 bp, Artificial Sequence
  Description of Artificial Sequence/note = Synthetic Construct
cgctgttgctagccccggggtgcttcaccatcccttgttggtttccctta

SEQ ID NO: 263 - DNA, 50 bp, Artificial Sequence
  Description of Artificial Sequence/note = Synthetic Construct
aagtcagcttcaaataaagaccctgcacaaagcctcggcccggtgaaaac

SEQ ID NO: 264 - DNA, 50 bp, Artificial Sequence
  Description of Artificial Sequence/note = Synthetic Construct
gacagccanacaatagacagcctgtcaataganatagccacacaataata

SEQ ID NO: 265 - DNA, 50 bp, Artificial Sequence
  Description of Artificial Sequence/note = Synthetic Construct
aagaatctgaacagcagcccttgagtcccagatcttccctctgacatagt

SEQ ID NO: 266 - DNA, 50 bp, Artificial Sequence
  Description of Artificial Sequence/note = Synthetic Construct
aatctacccacctgctttagccacarctggtkyytacccakggayacctc

SEQ ID NO: 267 - DNA, 50 bp, Artificial Sequence
  Description of Artificial Sequence/note = Synthetic Construct
aagaaacatawtcacattcaarggagtcccaatatggctatcagcagatt

SEQ ID NO: 268 - DNA, 50 bp, Artificial Sequence
  Description of Artificial Sequence/note = Synthetic Construct
agtggmaatctcatcagcccagggatctracaggagaaggtcttcctccc

SEQ ID NO: 269 - DNA, 50 bp, Artificial Sequence
  Description of Artificial Sequence/note = Synthetic Construct
yacatcmatagaaaaggtctgagagagycccagaatccctagccaggctg

SEQ ID NO: 270 - DNA, 50 bp, Artificial Sequence
  Description of Artificial Sequence/note = Synthetic Construct
gtcgcgctacgctgatangattnancataccctanatgctcggcgactgc

SEQ ID NO: 271 - DNA, 50 bp, Artificial Sequence
  Description of Artificial Sequence/note = Synthetic Construct
cactcagtgcgaamagcattatacctgggggcatttgttgaaaacawtta

SEQ ID NO: 272 - DNA, 50 bp, Artificial Sequence
  Description of Artificial Sequence/note = Synthetic Construct
tgaaagtggacttggattagttgtaaatgtatattgcaaactctagggca

SEQ ID NO: 273 - DNA, 50 bp, Artificial Sequence
  Description of Artificial Sequence/note = Synthetic Construct
ctgacacctacagctacagcaaacagtaaacacagtctaactcttagcca

SEQ ID NO: 274 - DNA, 50 bp, Artificial Sequence
  Description of Artificial Sequence/note = Synthetic Construct
accacagccactggaaagagtggggaaaatcccggaaaggagagagccag

SEQ ID NO: 275 - DNA, 50 bp, Artificial Sequence
  Description of Artificial Sequence/note = Synthetic Construct
acaaaaatatccagcacccaacaaggtaaaattcacaatgtctggcatcc

SEQ ID NO: 276 - DNA, 50 bp, Artificial Sequence
  Description of Artificial Sequence/note = Synthetic Construct
tcgtgaccttgggytaggcaawgatttcttagatatgacacmaaaagcac

SEQ ID NO: 277 - DNA, 50 bp, Artificial Sequence
  Description of Artificial Sequence/note = Synthetic Construct
aagctctgaataaatagcctttgcttgttctcatttggktggtcttcatt

SEQ ID NO: 278 - DNA, 50 bp, Artificial Sequence
  Description of Artificial Sequence/note = Synthetic Construct
cctcgctgcarcgagcaataaacccaacttgttcaaccacaggtgtgttc

SEQ ID NO: 279 - DNA, 50 bp, Artificial Sequence
  Description of Artificial Sequence/note = Synthetic Construct
acagcaaccaaaacgagattacggagtagactggacataagcaacacact

SEQ ID NO: 280 - DNA, 50 bp, Artificial Sequence
  Description of Artificial Sequence/note = Synthetic Construct
ataatgacaattttccaacagatggcagtaaagtgtcttgaggaaggggc

SEQ ID NO: 281 - DNA, 50 bp, Artificial Sequence
  Description of Artificial Sequence/note = Synthetic Construct
cctgtacttcttcaaatgataaaaagcttcatcgctaccttagttcacca

SEQ ID NO: 282 - DNA, 50 bp, Artificial Sequence
  Description of Artificial Sequence/note = Synthetic Construct
tgccttccaagcaatgaatatgctcaattnaaatcatatgctcgtgattg

SEQ ID NO: 283 - DNA, 50 bp, Artificial Sequence
  Description of Artificial Sequence/note = Synthetic Construct
gaaattgcctaatgacgcatttctcagaacgtatccccgtcgttaagcga

SEQ ID NO: 284 - DNA, 50 bp, Artificial Sequence
  Description of Artificial Sequence/note = Synthetic Construct
tcctgcaagctccattcatggtaagtgcyctatacaggtgtaccattttt

SEQ ID NO: 285 - DNA, 50 bp, Artificial Sequence
  Description of Artificial Sequence/note = Synthetic Construct
tgtgtctgtggctcgcgtttttcccggacatgccctaaagctggcttaat

SEQ ID NO: 286 - DNA, 50 bp, Artificial Sequence
  Description of Artificial Sequence/note = Synthetic Construct
cgtgttaatttcyattacatggrgagcccaggaacctgtggtcnntaaca

SEQ ID NO: 287 - DNA, 50 bp, Artificial Sequence
  Description of Artificial Sequence/note = Synthetic Construct
cctgtacttcttccccctaagctagctttggaataaaaagtcactttctt

SEQ ID NO: 288 - DNA, 50 bp, Artificial Sequence
  Description of Artificial Sequence/note = Synthetic Construct
cagactgaaggctgcactgtyggcttccctacttttgaggttttgggact

SEQ ID NO: 289 - DNA, 50 bp, Artificial Sequence
  Description of Artificial Sequence/note = Synthetic Construct
gnagggatggggactgcttttcgtnataagccttgtagnactatttgact

SEQ ID NO: 290 - DNA, 50 bp, Artificial Sequence
  Description of Artificial Sequence/note = Synthetic Construct
ctgggccccttagatcaggtatccagagatttttactcctccggtgctag

SEQ ID NO: 291 - DNA, 50 bp, Artificial Sequence
  Description of Artificial Sequence/note = Synthetic Construct
ttccttcccccactgtggaaaaagccagttttgcntcyatttgcaaattc

SEQ ID NO: 292 - DNA, 50 bp, Artificial Sequence
  Description of Artificial Sequence/note = Synthetic Construct
gggaatgtacctntgttgactttgctatttactatttgattagggcccag

SEQ ID NO: 293 - DNA, 50 bp, Artificial Sequence
  Description of Artificial Sequence/note = Synthetic Construct
acgttttctcaccgatatcacactgcatatgaacaagctaaatttgaagc

SEQ ID NO: 294 - DNA, 50 bp, Artificial Sequence
  Description of Artificial Sequence/note = Synthetic Construct
ttaaggtaggctaggctaagctatgatgttcggtaggttaggtgtattaa

SEQ ID NO: 295 - DNA, 50 bp, Artificial Sequence
  Description of Artificial Sequence/note = Synthetic Construct
ggtttctactgaatgtgtatcgctttcgcaccatcgtaaagttgaaaaat

SEQ ID NO: 296 - DNA, 50 bp, Artificial Sequence
  Description of Artificial Sequence/note = Synthetic Construct
gtttaccctcgtgatcgcgcggctgactgggarctgcggytcactgycgc

SEQ ID NO: 297 - DNA, 50 bp, Artificial Sequence
  Description of Artificial Sequence/note = Synthetic Construct
atctcccatctgctagcatttgattaataaagctgctttcctttcaccac

SEQ ID NO: 298 - DNA, 50 bp, Artificial Sequence
  Description of Artificial Sequence/note = Synthetic Construct
atgacagttgatgagcagttagttgcattcaaaggatattgcccatttcg

SEQ ID NO: 299 - DNA, 50 bp, Artificial Sequence
  Description of Artificial Sequence/note = Synthetic Construct
gcgcctgacagacctgttgctgcacacatctgtactcttcaatcaacaaa

SEQ ID NO: 300 - DNA, 50 bp, Artificial Sequence
  Description of Artificial Sequence/note = Synthetic Construct
accacccctggtcattaaggagctaccctgtctccattagahagagcagg

SEQ ID NO: 301 - DNA, 50 bp, Artificial Sequence
  Description of Artificial Sequence/note = Synthetic Construct
gagcagagccccagccgacccgcgatggacatgtagcatgagcaagaaat

SEQ ID NO: 302 - DNA, 50 bp, Artificial Sequence
  Description of Artificial Sequence/note = Synthetic Construct
aggggtagtggctgctccttatatctgctattcctatattctttagagtt

SEQ ID NO: 303 - DNA, 50 bp, Artificial Sequence
  Description of Artificial Sequence/note = Synthetic Construct
caataaagctcctcttcgccttgctcaccctccacttgtccgcgtacctc

SEQ ID NO: 304 - DNA, 50 bp, Artificial Sequence
  Description of Artificial Sequence/note = Synthetic Construct
tctcctctgagctgttctatcgctcaataaagctcctcttcatcttgctc

SEQ ID NO: 305 - DNA, 50 bp, Artificial Sequence
  Description of Artificial Sequence/note = Synthetic Construct
aggatggccagaggacaaagrgggcagagagacaatgggacwggatgacc

SEQ ID NO: 306 - DNA, 50 bp, Artificial Sequence
  Description of Artificial Sequence/note = Synthetic Construct
gcctgggacagtcctggtttatrcctgttgtcctggcgtaattattaata

SEQ ID NO: 307 - DNA, 50 bp, Artificial Sequence
  Description of Artificial Sequence/note = Synthetic Construct
gaggggnaaccacacaaaaagagnaggctaataagttggccaaaataagc

SEQ ID NO: 308 - DNA, 50 bp, Artificial Sequence
  Description of Artificial Sequence/note = Synthetic Construct
tttctcccgctgcaaaatctcggtgtsgatgtttggttttactgcgccgg

SEQ ID NO: 309 - DNA, 50 bp, Artificial Sequence
  Description of Artificial Sequence/note = Synthetic Construct
tctctgacccaggagtctcgtgtcttctgccagcatccatgaaactgtgg

SEQ ID NO: 310 - DNA, 50 bp, Artificial Sequence
  Description of Artificial Sequence/note = Synthetic Construct
tctctgacccaggagtctcatgtcttctgccagcatccatgaaactgtgg

SEQ ID NO: 311 - DNA, 50 bp, Artificial Sequence
  Description of Artificial Sequence/note = Synthetic Construct
tgcttggatgtcctgttgatagtagccttaattaaatgctntatgagaca

SEQ ID NO: 312 - DNA, 50 bp, Artificial Sequence
  Description of Artificial Sequence/note = Synthetic Construct
gtgtcgttttatctaaatcggcgcgaggaccaaggaccctggtgttcctc

SEQ ID NO: 313 - DNA, 50 bp, Artificial Sequence
  Description of Artificial Sequence/note = Synthetic Construct
ctccaaatggtgctgcagaccgaaccacacatagacacgccattcttcca

SEQ ID NO: 314 - DNA, 50 bp, Artificial Sequence
  Description of Artificial Sequence/note = Synthetic Construct
gagatsaaatcaaaatcattgacaggctcagggaaaatgccggcttcagc

SEQ ID NO: 315 - DNA, 50 bp, Artificial Sequence
  Description of Artificial Sequence/note = Synthetic Construct
tagacacaggnaagagacctgggaagcttnagtagccaccgtgtaagccc

SEQ ID NO: 316 - DNA, 50 bp, Artificial Sequence
  Description of Artificial Sequence/note = Synthetic Construct
ttcgctccaacctcaccctttgtgtccatgctccttaattttcttggtcg

SEQ ID NO: 317 - DNA, 50 bp, Artificial Sequence
  Description of Artificial Sequence/note = Synthetic Construct
ctragracccttaaaccagcctcrrgaraartcctaactgctgttnccta

SEQ ID NO: 318 - DNA, 50 bp, Artificial Sequence
  Description of Artificial Sequence/note = Synthetic Construct
cttctttctttggaatcccaactggccccatctcagganggtttggggya

SEQ ID NO: 319 - DNA, 50 bp, Artificial Sequence
  Description of Artificial Sequence/note = Synthetic Construct
ttcytttgcaataaattrctctatgctgcatctcctttgctgtgtgtctc

SEQ ID NO: 320 - DNA, 50 bp, Artificial Sequence
  Description of Artificial Sequence/note = Synthetic Construct
gtgtgtcttcccaggtcaatcctcacatttggcttccaataaacctttat

SEQ ID NO: 321 - DNA, 50 bp, Artificial Sequence
  Description of Artificial Sequence/note = Synthetic Construct
gtctcccggttcgcgarctgtwctttctctyattgtatgcacaataaact

SEQ ID NO: 322 - DNA, 50 bp, Artificial Sequence
  Description of Artificial Sequence/note = Synthetic Construct
taaatgacaccatrgggatgcaatcagcaaaatccagactgtgggaaact

SEQ ID NO: 323 - DNA, 50 bp, Artificial Sequence
  Description of Artificial Sequence/note = Synthetic Construct
atggagcagagctgccataccagccctggactgcctacctctagacttct

SEQ ID NO: 324 - DNA, 50 bp, Artificial Sequence
  Description of Artificial Sequence/note = Synthetic Construct
caagacatgatgctactccaagaataccgacggctccaggaacagcagtc

SEQ ID NO: 325 - DNA, 50 bp, Artificial Sequence
  Description of Artificial Sequence/note = Synthetic Construct
aaactcatttggcagcaaaacctgacctgaactgatatgaggctatttat

SEQ ID NO: 326 - DNA, 50 bp, Artificial Sequence
  Description of Artificial Sequence/note = Synthetic Construct
aatttaaggaggcactcactctcagggtcgtgcaagtgcagggtcggcat

SEQ ID NO: 327 - DNA, 50 bp, Artificial Sequence
  Description of Artificial Sequence/note = Synthetic Construct
gcccacctcctgtctccttgctggccggttttgcaataaagcctttcttt

SEQ ID NO: 328 - DNA, 50 bp, Artificial Sequence
  Description of Artificial Sequence/note = Synthetic Construct
tctggcattaagctggtcccccacytyyrcaggttttntgctggatataa

SEQ ID NO: 329 - DNA, 50 bp, Artificial Sequence
  Description of Artificial Sequence/note = Synthetic Construct
gctttcaacttgatgtcagtggattccttcgaatcagtaatgtctctatg

SEQ ID NO: 330 - DNA, 50 bp, Artificial Sequence
  Description of Artificial Sequence/note = Synthetic Construct
aatacggttcgtctgctcataactgttatacccgtgcgactgtcattagt

SEQ ID NO: 331 - DNA, 50 bp, Artificial Sequence
  Description of Artificial Sequence/note = Synthetic Construct
ctcaggctccagtatgagtngacactgcacagttrctgatcctgtattta

SEQ ID NO: 332 - DNA, 50 bp, Artificial Sequence
  Description of Artificial Sequence/note = Synthetic Construct
tcttgccaccacgnggagagagcctgcctgagaatgaagccaacacagag

SEQ ID NO: 333 - DNA, 50 bp, Artificial Sequence
  Description of Artificial Sequence/note = Synthetic Construct
cccttggaccagtctaaagcaccacattaacatcttatatgtagtccttg

SEQ ID NO: 334 - DNA, 50 bp, Artificial Sequence
  Description of Artificial Sequence/note = Synthetic Construct
cgctgcatacctgtgtctgagtactcatttcatccatcggtcggccaggg

SEQ ID NO: 335 - DNA, 50 bp, Artificial Sequence
  Description of Artificial Sequence/note = Synthetic Construct
acacagacgtggcttctgtttgtaagtccctattaaatgtttctttctga

SEQ ID NO: 336 - DNA, 50 bp, Artificial Sequence
  Description of Artificial Sequence/note = Synthetic Construct
tccttctgcgtttgggggtcattttgcatatacggccctttcacgaaaca

SEQ ID NO: 337 - DNA, 50 bp, Artificial Sequence
  Description of Artificial Sequence/note = Synthetic Construct
ttcgttttacaccgaaggctgcatctccccggtttgcaaactgttcactg

SEQ ID NO: 338 - DNA, 50 bp, Artificial Sequence
  Description of Artificial Sequence/note = Synthetic Construct
cagttcatttcagcaaaccttcagaggggacagaggggaagctttccttt

SEQ ID NO: 339 - DNA, 50 bp, Artificial Sequence
  Description of Artificial Sequence/note = Synthetic Construct
taatcattctcctctgtgattcccccatgctatgcacgttaaaataaatt

SEQ ID NO: 340 - DNA, 50 bp, Artificial Sequence
  Description of Artificial Sequence/note = Synthetic Construct
tgccttttgtcagttgatttttcagcgaaccttcagagggcgaaggggaa

SEQ ID NO: 341 - DNA, 50 bp, Artificial Sequence
  Description of Artificial Sequence/note = Synthetic Construct
ctctttctttattgcaatgccatggtctttgtctgtgcagcgggcaggaa

SEQ ID NO: 342 - DNA, 50 bp, Artificial Sequence
  Description of Artificial Sequence/note = Synthetic Construct
gtagaagccccaaacccymttggcgcaactcwctctcttgagtatgcccg

SEQ ID NO: 343 - DNA, 50 bp, Artificial Sequence
  Description of Artificial Sequence/note = Synthetic Construct
tcccccctccagaccttcacttccccagctcctcccacaattgtataagg

SEQ ID NO: 344 - DNA, 50 bp, Artificial Sequence
  Description of Artificial Sequence/note = Synthetic Construct
tctctgttaaaataactggtgtggtttctgtcttctcctgactggaccct

SEQ ID NO: 345 - DNA, 50 bp, Artificial Sequence
  Description of Artificial Sequence/note = Synthetic Construct
tctttgaagagagagcgcctttggtctatgccagagactatctcttccca

SEQ ID NO: 346 - DNA, 50 bp, Artificial Sequence
  Description of Artificial Sequence/note = Synthetic Construct
gtgcattgtgaatctccaagaggggaaatatagtatgcagtrtttcccaa

SEQ ID NO: 347 - DNA, 50 bp, Artificial Sequence
  Description of Artificial Sequence/note = Synthetic Construct
ttaacatctctgaaatcgggatgcatcttacaatcgatggcatgtcatag

SEQ ID NO: 348 - DNA, 50 bp, Artificial Sequence
  Description of Artificial Sequence/note = Synthetic Construct
acaacggcagagttgagtagttgcgacagagaccgtatggcccgcaaagc

SEQ ID NO: 349 - DNA, 50 bp, Artificial Sequence; identical to SEQ ID NO: 348
  Description of Artificial Sequence/note = Synthetic Construct

SEQ ID NO: 350 - DNA, 50 bp, Artificial Sequence
  Description of Artificial Sequence/note = Synthetic Construct
atctgctcttcgccttgcccagagaccccactgtgaattaccatttggag

SEQ ID NO: 351 - DNA, 50 bp, Artificial Sequence
  Description of Artificial Sequence/note = Synthetic Construct
gtattggcttcgcatcaggcagcagnnagcccattgattgcttrgtaaca

SEQ ID NO: 352 - DNA, 50 bp, Artificial Sequence
  Description of Artificial Sequence/note = Synthetic Construct
ataccctcttggtgtgtgtgtggcatcatcagtcttaacatccaaaccaa

SEQ ID NO: 353 - DNA, 50 bp, Artificial Sequence
  Description of Artificial Sequence/note = Synthetic Construct
gccctaaggcatccattgtatgtaatgaattaacttctctcctatgcatc

SEQ ID NO: 354 - DNA, 50 bp, Artificial Sequence
  Description of Artificial Sequence/note = Synthetic Construct
catctgtccagtgttgggtgtcatgtgtttarccatccccataaccctag

SEQ ID NO: 355 - DNA, 50 bp, Artificial Sequence
  Description of Artificial Sequence/note = Synthetic Construct
tataaagccaacctcctctgctcagctcatyggaacactcattctatttt

SEQ ID NO: 356 - DNA, 50 bp, Artificial Sequence
  Description of Artificial Sequence/note = Synthetic Construct
tgtggtattaaaatttcatggngggggggggtgattaggaaaaaaatgtc

SEQ ID NO: 357 - DNA, 50 bp, Artificial Sequence
  Description of Artificial Sequence/note = Synthetic Construct
ttctacttatcactagagacagaaactaaaaaccatggcttcaggctgct

SEQ ID NO: 358 - DNA, 50 bp, Artificial Sequence
  Description of Artificial Sequence/note = Synthetic Construct
acttaataatggccccaaagcgcaagagtagtgatgctggcatattgtta

SEQ ID NO: 359 - DNA, 50 bp, Artificial Sequence
  Description of Artificial Sequence/note = Synthetic Construct
ctactgacagcaggggagatagggcatacgtgggtagagcggataattcc

SEQ ID NO: 360 - DNA, 50 bp, Artificial Sequence
  Description of Artificial Sequence/note = Synthetic Construct
ccctggaaggctttcaggtcagcttcaacttactggccagagttgtgctg

SEQ ID NO: 361 - DNA, 50 bp, Artificial Sequence
  Description of Artificial Sequence/note = Synthetic Construct
cctctttgcagacagccccttctctgctgtgctgcccgttgcaaccttgc

SEQ ID NO: 362 - DNA, 50 bp, Artificial Sequence
  Description of Artificial Sequence/note = Synthetic Construct
gcacgtagccccctccagtacaaccctataaaacttccctccagcccctg

SEQ ID NO: 363 - DNA, 50 bp, Artificial Sequence
  Description of Artificial Sequence/note = Synthetic Construct
gaaagaacctgggtccttgatgatatcgttgagccgctgaattaaccaac

SEQ ID NO: 364 - DNA, 50 bp, Artificial Sequence
  Description of Artificial Sequence/note = Synthetic Construct
atcagacgcaragacaacagccttacagagactgcttaaccagctcccac

SEQ ID NO: 365 - DNA, 50 bp, Artificial Sequence
  Description of Artificial Sequence/note = Synthetic Construct
tcatatctttttccttgatcagcccccaaatcccttraacccccttcaca

SEQ ID NO: 366 - DNA, 50 bp, Artificial Sequence
  Description of Artificial Sequence/note = Synthetic Construct
ctcttttttgcctttaaaaatccacttgtaactgctgctaattggagtgt

SEQ ID NO: 367 - DNA, 50 bp, Artificial Sequence
  Description of Artificial Sequence/note = Synthetic Construct
gagtgccctgtatgtaagtcctaataaactcatctacttatcaagctgga

SEQ ID NO: 368 - DNA, 50 bp, Artificial Sequence
  Description of Artificial Sequence/note = Synthetic Construct
agccgcaagcctattaaaccttgcctgagaaaatcggtttggcctggtgt

SEQ ID NO: 369 - DNA, 50 bp, Artificial Sequence
  Description of Artificial Sequence/note = Synthetic Construct
atttttcctrgrtgtgccctcaagctggctcagtaaacctcgatgntttg

SEQ ID NO: 370 - DNA, 50 bp, Artificial Sequence
  Description of Artificial Sequence/note = Synthetic Construct
ctganaggataaagatacctcgtgacaaagcctcctgggtataatactcc

SEQ ID NO: 371 - DNA, 50 bp, Artificial Sequence
  Description of Artificial Sequence/note = Synthetic Construct
aaaatggcttccctgggttcttccctttttaggcccacttgttagtctcc

SEQ ID NO: 372 - DNA, 50 bp, Artificial Sequence
  Description of Artificial Sequence/note = Synthetic Construct
tccaattacaggtgtgacgttttcattcctcatcattatcccacaacgcc

SEQ ID NO: 373 - DNA, 50 bp, Artificial Sequence
  Description of Artificial Sequence/note = Synthetic Construct
tcggtgtattgacttgccgcgcatcgggcaacaaacctattacggtcaca

SEQ ID NO: 374 - DNA, 50 bp, Artificial Sequence
  Description of Artificial Sequence/note = Synthetic Construct
ctgccctatcctgcttccctcactcccttacaagtttctcctgagagcac

SEQ ID NO: 375 - DNA, 50 bp, Artificial Sequence
  Description of Artificial Sequence/note = Synthetic Construct
tctttggaatctgtgyttccngggtggnccatcntcaaactttgcacttg

SEQ ID NO: 376 - DNA, 50 bp, Artificial Sequence
  Description of Artificial Sequence/note = Synthetic Construct
cccgctcctgctccctccccttttatctttcacaggntttcccctaataa

SEQ ID NO: 377 - DNA, 50 bp, Artificial Sequence
  Description of Artificial Sequence/note = Synthetic Construct
cttcaaraaaaatcygacatcataaaaaccccgtgcagactctcagggct

SEQ ID NO: 378 - DNA, 50 bp, Artificial Sequence
  Description of Artificial Sequence/note = Synthetic Construct
gtaggcagaattctaagatggcccccaagattcccaccccctggtgtaca

SEQ ID NO: 379 - DNA, 50 bp, Artificial Sequence
  Description of Artificial Sequence/note = Synthetic Construct
tagccaacggaatgtaagcagaagtgatgtgcgccacttccaggcctggc

SEQ ID NO: 380 - DNA, 50 bp, Artificial Sequence
  Description of Artificial Sequence/note = Synthetic Construct
cctgagtcactacntggaggagagccacccacacccgaccagaacccnca

SEQ ID NO: 381 - DNA, 50 bp, Artificial Sequence
  Description of Artificial Sequence/note = Synthetic Construct
tcrgctrgggrcrgtcagagargagntcagccgctggayngccaaactcc

SEQ ID NO: 382 - DNA, 50 bp, Artificial Sequence
  Description of Artificial Sequence/note = Synthetic Construct
tgtccrtcattnctggcatngtcaggactaggtamggtctcgdccaactg

SEQ ID NO: 383 - DNA, 50 bp, Artificial Sequence
  Description of Artificial Sequence/note = Synthetic Construct
gccccccaaagatgtccatgccctaatccctggaacctgtgaatatgtta

SEQ ID NO: 384 - DNA, 50 bp, Artificial Sequence
  Description of Artificial Sequence/note = Synthetic Construct
cactggctggtcggcaactgtttacagcactctcctgggagtctgtaagc

SEQ ID NO: 385 - DNA, 50 bp, Artificial Sequence
  Description of Artificial Sequence/note = Synthetic Construct
tttccaaagatggccgcaacaatatctcccatcccacatgctcttcttac

SEQ ID NO: 386 - DNA, 50 bp, Artificial Sequence
  Description of Artificial Sequence/note = Synthetic Construct
gcccatttccaggcataaatactatttacctcagtctctactgttcttct

SEQ ID NO: 387 - DNA, 50 bp, Artificial Sequence
  Description of Artificial Sequence/note = Synthetic Construct
ctcgcctcactgtgcccaccaatccaaagctattatgtcataaactctgc

SEQ ID NO: 388 - DNA, 50 bp, Artificial Sequence
  Description of Artificial Sequence/note = Synthetic Construct
caaagaatcctgcgtcaaaatcgagagaacgaacaagccttcatcgccat

SEQ ID NO: 389 - DNA, 50 bp, Artificial Sequence
  Description of Artificial Sequence/note = Synthetic Construct
aataaaaaggctggacaagatatatggtggagggatgcacatacaaagag

SEQ ID NO: 390 - DNA, 50 bp, Artificial Sequence
  Description of Artificial Sequence/note = Synthetic Construct
caggcgtctccacggagtccaatgaaaaactcgaagccagcgacaagcaa

SEQ ID NO: 391 - DNA, 50 bp, Artificial Sequence
  Description of Artificial Sequence/note = Synthetic Construct
ctcatagctcctataatgccattgaacaccagtgagagacgattagacgt

SEQ ID NO: 392 - DNA, 50 bp, Artificial Sequence
  Description of Artificial Sequence/note = Synthetic Construct
accgccactgctacacatcttatcgaatgactcacgagttctccttcact

SEQ ID NO: 393 - DNA, 50 bp, Artificial Sequence
  Description of Artificial Sequence/note = Synthetic Construct
atccactgagctggtgcgtaccttaaaataaataacaatcctcctgtatt

SEQ ID NO: 394 - DNA, 50 bp, Artificial Sequence
  Description of Artificial Sequence/note = Synthetic Construct
ctcaatttgttttctcccctcctttgcctatctctatctaacaacctcta

SEQ ID NO: 395 - DNA, 50 bp, Artificial Sequence
  Description of Artificial Sequence/note = Synthetic Construct
atagaggcagtagtaacccgaaacactaccatgctattgacggcattaac

SEQ ID NO: 396 - DNA, 50 bp, Artificial Sequence
  Description of Artificial Sequence/note = Synthetic Construct
caaanatgtgtggacctggttatctctgaccttgcrctgctcacgacaca

SEQ ID NO: 397 - DNA, 50 bp, Artificial Sequence
  Description of Artificial Sequence/note = Synthetic Construct
ggctataggcntycctcagtctacagtcctcagtaagacttctgaataaa

SEQ ID NO: 398 - DNA, 50 bp, Artificial Sequence
  Description of Artificial Sequence/note = Synthetic Construct
ccagaccagtggctttcaaactttttttgactatgacccacagtaagaaa

SEQ ID NO: 399 - DNA, 50 bp, Artificial Sequence
  Description of Artificial Sequence/note = Synthetic Construct
aagcaccaaactgagactttctccttgatgtaatcagaaggattgaaaga

SEQ ID NO: 400 - DNA, 50 bp, Artificial Sequence
  Description of Artificial Sequence/note = Synthetic Construct
ttacccaatcctaatcaagcccctacattgaaagacctgccttaaatcag

SEQ ID NO: 401 - DNA, 50 bp, Artificial Sequence
  Description of Artificial Sequence/note = Synthetic Construct
cttcttgctgttgctaatctctgggttgcctcaccattgnttccctgttt

SEQ ID NO: 402 - DNA, 50 bp, Artificial Sequence
  Description of Artificial Sequence/note = Synthetic Construct
ccccggccgacatcttgactgcaacctcatgagagaccctgagccagaac

SEQ ID NO: 403 - DNA, 50 bp, Artificial Sequence
  Description of Artificial Sequence/note = Synthetic Construct
acaccccccccstacvcccacmccccctgtgatattgttcgtaatatcca

SEQ ID NO: 404 - DNA, 50 bp, Artificial Sequence
  Description of Artificial Sequence/note = Synthetic Construct
tttaaatatttagacatatggtatgtgggcctccatttgtactcttgccc

SEQ ID NO: 405 - DNA, 50 bp, Artificial Sequence
  Description of Artificial Sequence/note = Synthetic Construct
gcacaggagggggaagtagcagcanatatgctatgtatttgccatccctg

SEQ ID NO: 406 - DNA, 50 bp, Artificial Sequence
  Description of Artificial Sequence/note = Synthetic Construct
taggtgcaagcatctgactacttcattatgtcttctagtgtagtcatgcc

SEQ ID NO: 407 - DNA, 50 bp, Artificial Sequence
  Description of Artificial Sequence/note = Synthetic Construct
tccatggttcctctggtgtgcagtctccctcattgcaataagtcaataaa

SEQ ID NO: 408 - DNA, 50 bp, Artificial Sequence
  Description of Artificial Sequence/note = Synthetic Construct
tgaagyggttgctttggataggaatcyggccrcttccccattactagttt

SEQ ID NO: 409 - DNA, 50 bp, Artificial Sequence
  Description of Artificial Sequence/note = Synthetic Construct
ggattgacagcagatcamgggaagtgattatacccctttacaatgccttg

SEQ ID NO: 410 - DNA, 50 bp, Artificial Sequence
  Description of Artificial Sequence/note = Synthetic Construct
gtgggatggacagggatgggagggactgacttttcactgtataccttttt

SEQ ID NO: 411 - DNA, 50 bp, Artificial Sequence
  Description of Artificial Sequence/note = Synthetic Construct
tggaccctccagaccagcccatctgccagctgaataccactgagtgacct

SEQ ID NO: 412 - DNA, 50 bp, Artificial Sequence
  Description of Artificial Sequence/note = Synthetic Construct
gggacagaaattgtgcactcggggagctcggattttaaggcagtagcttg

SEQ ID NO: 413 - DNA, 50 bp, Artificial Sequence
  Description of Artificial Sequence/note = Synthetic Construct
ccagaaaccacctccccacaagcccactagaaacaaacatctgacagaga

SEQ ID NO: 414 - DNA, 50 bp, Artificial Sequence
  Description of Artificial Sequence/note = Synthetic Construct
tagccnataaaatactcttaacagctccagnaacagttgcatcagcagaa

SEQ ID NO: 415 - DNA, 50 bp, Artificial Sequence
  Description of Artificial Sequence/note = Synthetic Construct
tttaaaacatggccgcaaattctttgacactcctctcattgagangtggg

SEQ ID NO: 416 - DNA, 50 bp, Artificial Sequence
  Description of Artificial Sequence/note = Synthetic Construct
cttgcttcctctctcaccatgtgatctctgcacacgctggctccccttcc

SEQ ID NO: 417 - DNA, 50 bp, Artificial Sequence
  Description of Artificial Sequence/note = Synthetic Construct
gaattcgtctcaaagtgtggcgtttctctataactcgctcggttacaaca

SEQ ID NO: 418 - DNA, 50 bp, Artificial Sequence
  Description of Artificial Sequence/note = Synthetic Construct
ggtctggaaaccatgtcatatgaggaacggttgaaggaactggggatgtt

SEQ ID NO: 419 - DNA, 50 bp, Artificial Sequence
  Description of Artificial Sequence/note = Synthetic Construct
tgccatttacgtgggataaagcttgtttacccttaaaggtattgtgtgtg

SEQ ID NO: 420 - DNA, 50 bp, Artificial Sequence
  Description of Artificial Sequence/note = Synthetic Construct
accttttgtcggaactcggagttatgaacgaccctcaccataccgatgct

SEQ ID NO: 421 - DNA, 50 bp, Artificial Sequence
  Description of Artificial Sequence/note = Synthetic Construct
tatngcctcccaaggtgactactttgaaggggacaacactcatttggatg

SEQ ID NO: 422 - DNA, 50 bp, Artificial Sequence
  Description of Artificial Sequence/note = Synthetic Construct
ttactgagacactaagggcgccgtgaaccgagaaagtttgggaacctctg

SEQ ID NO: 423 - DNA, 50 bp, Artificial Sequence
  Description of Artificial Sequence/note = Synthetic Construct
gttctccagccctcccggagattctgtgagctacccaatatcctttaata

SEQ ID NO: 424 - DNA, 50 bp, Artificial Sequence
  Description of Artificial Sequence/note = Synthetic Construct
cgggcngattggtgagatccntctcctacacgaggccagtctgacaagac

SEQ ID NO: 425 - DNA, 50 bp, Artificial Sequence
  Description of Artificial Sequence/note = Synthetic Construct
ctcttatggactatctccgtgcaattgcccataatctatccctgtaatat

SEQ ID NO: 426 - DNA, 50 bp, Artificial Sequence
  Description of Artificial Sequence/note = Synthetic Construct
aggggtctggggagtcatgccctacaaaccataaattctcatcagatggg

SEQ ID NO: 427 - DNA, 50 bp, Artificial Sequence
  Description of Artificial Sequence/note = Synthetic Construct
acctttcgcgtttcagttaacaaaccatttaaggaccatttgaggaagga

SEQ ID NO: 428 - DNA, 50 bp, Artificial Sequence
  Description of Artificial Sequence/note = Synthetic Construct
tgctcatgctgcttgctgtgycatgagtaataaagtcctttgtctctgac

SEQ ID NO: 429 - DNA, 50 bp, Artificial Sequence
  Description of Artificial Sequence/note = Synthetic Construct
tgctcaagctactttacaaaagccaaactgctctgccatgcccagcggag

SEQ ID NO: 430 - DNA, 50 bp, Artificial Sequence
  Description of Artificial Sequence/note = Synthetic Construct
ggaagcagtatggtatagtggaaagaacaactggactaggagtcaggaga

SEQ ID NO: 431 - DNA, 50 bp, Artificial Sequence
  Description of Artificial Sequence/note = Synthetic Construct
ccagctgtcaagtcatccccagcctctnncagycmtccccagccttcaag

SEQ ID NO: 432 - DNA, 50 bp, Artificial Sequence
  Description of Artificial Sequence/note = Synthetic Construct
ccacttcccctttgaccttctctgccatgttatgatgcagcatgaaagcc

SEQ ID NO: 433 - DNA, 50 bp, Artificial Sequence
  Description of Artificial Sequence/note = Synthetic Construct
tttgagaactgaactaaaggatagaccactacccaggtcccagactggcc

SEQ ID NO: 434 - DNA, 50 bp, Artificial Sequence
  Description of Artificial Sequence/note = Synthetic Construct
artgctaatttttctttgcagcaccgaggaacaagcattctgtttctaaa

SEQ ID NO: 435 - DNA, 50 bp, Artificial Sequence
  Description of Artificial Sequence/note = Synthetic Construct
tctctggagtctgtgtttcctgaatggccattcccagcttttnacttgaa

SEQ ID NO: 436 - DNA, 50 bp, Artificial Sequence
  Description of Artificial Sequence/note = Synthetic Construct
tggagtgatgcagccataagccaaggaatgccagcagccaagccaccaga

SEQ ID NO: 437 - DNA, 50 bp, Artificial Sequence
  Description of Artificial Sequence/note = Synthetic Construct
gtgggtttgttataaaagnaagttcggcccccttttgctctctcnctctc

SEQ ID NO: 438 - DNA, 50 bp, Artificial Sequence
  Description of Artificial Sequence/note = Synthetic Construct
atctttacgtcatatacatttccatgtctcaggaggctagggctttttac

SEQ ID NO: 439 - DNA, 50 bp, Artificial Sequence
  Description of Artificial Sequence/note = Synthetic Construct
taaaaacccagtggataggtnaaacagcagattaganacagctgaagaga

SEQ ID NO: 440 - DNA, 50 bp, Artificial Sequence
  Description of Artificial Sequence/note = Synthetic Construct
actgaaaggaaatatacaccaaaatgttaacagtggttatctctgggtgg

SEQ ID NO: 441 - DNA, 50 bp, Artificial Sequence
  Description of Artificial Sequence/note = Synthetic Construct
tagaagaaatagctgaccgtgggaatgttgacactgccgccatttgagag

SEQ ID NO: 442 - DNA, 50 bp, Artificial Sequence
  Description of Artificial Sequence/note = Synthetic Construct
agaccaaatccttcatccagataaggggtagccaataggaacctcaaaag

SEQ ID NO: 443 - DNA, 50 bp, Artificial Sequence
  Description of Artificial Sequence/note = Synthetic Construct
ccggctaaataaacggactcttaattcgtctcaaagtgtggcgttttctc

SEQ ID NO: 444 - DNA, 50 bp, Artificial Sequence
  Description of Artificial Sequence/note = Synthetic Construct
tccacagttcctggctcataactcccatagcccttgttacagtcttttgt

SEQ ID NO: 445 - DNA, 50 bp, Artificial Sequence
  Description of Artificial Sequence/note = Synthetic Construct
ccacaagttgctgcccctagagactcaaagtccttttcctttgtcttgtc

SEQ ID NO: 446 - DNA, 50 bp, Artificial Sequence
  Description of Artificial Sequence/note = Synthetic Construct
agtttcttttgtcttaagttttcatttctgcgttcgtcccccttcgttca

SEQ ID NO: 447 - DNA, 50 bp, Artificial Sequence
  Description of Artificial Sequence/note = Synthetic Construct
aggcggttgtataaggcagatatctggatcgaccacattgaggaactggg

SEQ ID NO: 448 - DNA, 50 bp, Artificial Sequence
  Description of Artificial Sequence/note = Synthetic Construct
gcctttcatctatccgagtgtcantgtgttgtgtcccgccatcaaaagaa

SEQ ID NO: 449 - DNA, 50 bp, Artificial Sequence
  Description of Artificial Sequence/note = Synthetic Construct
aagagtaaacatcactcaaggactttacctcctcttctggggaaggggtt

SEQ ID NO: 450 - DNA, 50 bp, Artificial Sequence
  Description of Artificial Sequence/note = Synthetic Construct
aaataccccnaataattgatgtcaaaactgacgtcaagacanaaaggggt

SEQ ID NO: 451 - DNA, 50 bp, Artificial Sequence
  Description of Artificial Sequence/note = Synthetic Construct
taagtcccaactcagggatttaggtccacgtaacctcctgaccgactaac

SEQ ID NO: 452 - DNA, 50 bp, Artificial Sequence
  Description of Artificial Sequence/note = Synthetic Construct
tctccgatgagttctttcctccagcaagatccaatatcctaagtcccaca

SEQ ID NO: 453 - DNA, 50 bp, Artificial Sequence
  Description of Artificial Sequence/note = Synthetic Construct
attttccctttcttgagaccccaataggcagcaggtagacatgagcatgg

SEQ ID NO: 454 - DNA, 50 bp, Artificial Sequence
  Description of Artificial Sequence/note = Synthetic Construct
taataaactgtctgaatctaaaagtggctcgttgtatctttaccagccga

SEQ ID NO: 455 - DNA, 50 bp, Artificial Sequence
  Description of Artificial Sequence/note = Synthetic Construct
caccgagctagctgcaggagtttttttttttcgtaccccagtggcgcctg

SEQ ID NO: 456 - DNA, 50 bp, Artificial Sequence
  Description of Artificial Sequence/note = Synthetic Construct
ctttagccctaggggaactgtcggacctgaactctgcagggcggtcttgc

SEQ ID NO: 457 - DNA, 50 bp, Artificial Sequence
  Description of Artificial Sequence/note = Synthetic Construct
aagaaacaaataacatacaatggagctccaatacgtctggcagcagactt

SEQ ID NO: 458 - DNA, 50 bp, Artificial Sequence
  Description of Artificial Sequence/note = Synthetic Construct
catgtcagacccgacaccaagagggatcccctcggctaagtctccccatt

SEQ ID NO: 459 - DNA, 50 bp, Artificial Sequence
  Description of Artificial Sequence/note = Synthetic Construct
cccattcgggacgggcagcgctctgattgtttactagagccgaggcaaac

SEQ ID NO: 460 - DNA, 50 bp, Artificial Sequence
  Description of Artificial Sequence/note = Synthetic Construct
aaaggggtggggatggagctgtaaaggagcagagtttttgtatgttattg

SEQ ID NO: 461 - DNA, 50 bp, Artificial Sequence
  Description of Artificial Sequence/note = Synthetic Construct
cacaaaagtaggccaggacctgcatgctaaacctaaacagggtgactgcc

SEQ ID NO: 462 - DNA, 50 bp, Artificial Sequence
  Description of Artificial Sequence/note = Synthetic Construct
cacaggaaggggaatatcacactctggggactgtggtggggtcgggggag

SEQ ID NO: 463 - DNA, 50 bp, Artificial Sequence
  Description of Artificial Sequence/note = Synthetic Construct
aacacatggacacaggaaggggaacatcacactctggggactgttgtggg

SEQ ID NO: 464 - DNA, 50 bp, Artificial Sequence
  Description of Artificial Sequence/note = Synthetic Construct
aacacatggacacaggaaggggaacatcacacaccggggcctgttgtggg

SEQ ID NO: 465 - DNA, 50 bp, Artificial Sequence
  Description of Artificial Sequence/note = Synthetic Construct
gaacacttggacacaggaaggggaacatcacacaccggggcctgttgtgg

SEQ ID NO: 466 - DNA, 50 bp, Artificial Sequence
  Description of Artificial Sequence/note = Synthetic Construct
gagaaatacctaatgtaaatgacgagttgatgggtgcagcaaaccaacat

SEQ ID NO: 467 - DNA, 50 bp, Artificial Sequence
  Description of Artificial Sequence/note = Synthetic Construct
aggacaaatacctaatgcatgcggggcttaaaacctagatgacgggttga

SEQ ID NO: 468 - DNA, 50 bp, Artificial Sequence
  Description of Artificial Sequence/note = Synthetic Construct
atagctaatgcatgctgggcttaatacctaggtgatgggttgataggtgc

SEQ ID NO: 469 - DNA, 50 bp, Artificial Sequence
  Description of Artificial Sequence/note = Synthetic Construct
cttaatacctgggtgatgaaataatctgtacaacaaacccccatgacaca

SEQ ID NO: 470 - DNA, 50 bp, Artificial Sequence
  Description of Artificial Sequence/note = Synthetic Construct
tacctgggtgatgaaataatctgtacaacaaacccccatgacacaagttt

SEQ ID NO: 471 - DNA, 50 bp, Artificial Sequence
  Description of Artificial Sequence/note = Synthetic Construct
gggagaggagcagaaaagataactattgggtactgggcttaatacctggg

SEQ ID NO: 472 - DNA, 50 bp, Artificial Sequence
  Description of Artificial Sequence/note = Synthetic Construct
tgggtgatgggatcattcgtaccccaaacctcagcatcacgcaatatacc

SEQ ID NO: 473 - DNA, 50 bp, Artificial Sequence
  Description of Artificial Sequence/note = Synthetic Construct
atctcagaaatcaccactaaagaacttatccatgtaaccaaaaaccacct

SEQ ID NO: 474 - DNA, 50 bp, Artificial Sequence
  Description of Artificial Sequence/note = Synthetic Construct
ktacactaaaagcccagacttcaccactacgcaatatatccatgtaacaa

SEQ ID NO: 475 - DNA, 50 bp, Artificial Sequence
  Description of Artificial Sequence/note = Synthetic Construct
attctccatgatgtgcttatttcacattgcatgcctgtatcaaaacatct

SEQ ID NO: 476 - DNA, 50 bp, Artificial Sequence
  Description of Artificial Sequence/note = Synthetic Construct
gctgggaagggtagtggggtgggggggaagtggggatggttaatgggtac

SEQ ID NO: 477 - DNA, 50 bp, Artificial Sequence
  Description of Artificial Sequence/note = Synthetic Construct
ggagggggggaatgaagagaggttggttaatgggtacaaaaatacagtta

SEQ ID NO: 478 - DNA, 50 bp, Artificial Sequence
  Description of Artificial Sequence/note = Synthetic Construct
gaggacttgaaatgttcccaacacatagaaatgataaatactcgaggtga

SEQ ID NO: 479 - DNA, 50 bp, Artificial Sequence
  Description of Artificial Sequence/note = Synthetic Construct
tgggaagggtagggggaagggggggatagggagagatttgttaaaggata

SEQ ID NO: 480 - DNA, 50 bp, Artificial Sequence
  Description of Artificial Sequence/note = Synthetic Construct
ataggaggaataagttctggtgttctattgcacagtagggtgactatagt

SEQ ID NO: 481 - DNA, 50 bp, Artificial Sequence
  Description of Artificial Sequence/note = Synthetic Construct
atggggagatgttggtcaaagggtacaaagtttcagttagacaggaggaa

SEQ ID NO: 482 - DNA, 50 bp, Artificial Sequence
  Description of Artificial Sequence/note = Synthetic Construct
tgctnatggtcccatgactggccactctgtgaacacagtaaacaagtttg

SEQ ID NO: 483 - DNA, 50 bp, Artificial Sequence
  Description of Artificial Sequence/note = Synthetic Construct
gaaatggggagttgctgttcaatgggtataaagtttcagttatgcaagat

SEQ ID NO: 484 - DNA, 50 bp, Artificial Sequence
  Description of Artificial Sequence/note = Synthetic Construct
gggtatagagtttcagttttgcaagatgaaaaagttctggagatcggttg

SEQ ID NO: 485 - DNA, 50 bp, Artificial Sequence
  Description of Artificial Sequence/note = Synthetic Construct
tggtgatggttgcacaacamtgtgaatgtacttaatgccactgaattgta

SEQ ID NO: 486 - DNA, 50 bp, Artificial Sequence
  Description of Artificial Sequence/note = Synthetic Construct
agggggaatggggagtgactgcttaatgggtacggggtttccttttgggg

SEQ ID NO: 487 - DNA, 50 bp, Artificial Sequence
  Description of Artificial Sequence/note = Synthetic Construct
ggaatggggagtgactgctaatgggtacggggtttcttttgggggtgatg

SEQ ID NO: 488 - DNA, 50 bp, Artificial Sequence
  Description of Artificial Sequence/note = Synthetic Construct
ggtgggggnaggggattgactacaaaggggcatgagggaactttttgggg

SEQ ID NO: 489 - DNA, 50 bp, Artificial Sequence
  Description of Artificial Sequence/note = Synthetic Construct
atagtggttacctttggggagggttattgactgggaaggggcatgaggga

SEQ ID NO: 490 - DNA, 50 bp, Artificial Sequence
  Description of Artificial Sequence/note = Synthetic Construct
gactggaaggaaatacaccaaaatgttaacagtggttatctctgggtggt

SEQ ID NO: 491 - DNA, 50 bp, Artificial Sequence
  Description of Artificial Sequence/note = Synthetic Construct
ttgatagtgggggaggctgtgcatgtgtgggggcagggggtatatgggaa

SEQ ID NO: 492 - DNA, 50 bp, Artificial Sequence
  Description of Artificial Sequence/note = Synthetic Construct
acccataaccccagtctaatcatgagaaaacatcagacaaacccaaattg

SEQ ID NO: 493 - DNA, 50 bp, Artificial Sequence
  Description of Artificial Sequence/note = Synthetic Construct
agaggagaggtggaaggaagtatgagagtgctaatntcctcatctttcat

SEQ ID NO: 494 - DNA, 50 bp, Artificial Sequence
  Description of Artificial Sequence/note = Synthetic Construct
agacccagggttcaggcctgtcccagtagaccccagcactaggctagtcc

SEQ ID NO: 495 - DNA, 50 bp, Artificial Sequence
  Description of Artificial Sequence/note = Synthetic Construct
aagaaggaatcttggaacatcaggaaggaagaaagaacatagtaagaagc

SEQ ID NO: 496 - DNA, 50 bp, Artificial Sequence
  Description of Artificial Sequence/note = Synthetic Construct
ggcagaaactggaggggagtcgacacctggaagaagggaatwgcacggag

SEQ ID NO: 497 - DNA, 50 bp, Artificial Sequence; identical to SEQ ID NO: 294
  Description of Artificial Sequence/note = Synthetic Construct

SEQ ID NO: 498 - DNA, 50 bp, Artificial Sequence
  Description of Artificial Sequence/note = Synthetic Construct
aggcaaccccatcaagaacttangcgaaaaaagatgtaggatcacaaagt

SEQ ID NO: 499 - DNA, 50 bp, Artificial Sequence
  Description of Artificial Sequence/note = Synthetic Construct
tcggatggaacgcagcattaaagtcacccatatgatcaatgaaggattac

SEQ ID NO: 500 - DNA, 50 bp, Artificial Sequence
  Description of Artificial Sequence/note = Synthetic Construct
cctcacttcatctcatcacgtaggcattttatcatctcacatcatcacaa

SEQ ID NO: 501 - DNA, 50 bp, Artificial Sequence
  Description of Artificial Sequence/note = Synthetic Construct
atcgacgaagataacataaaactcataatacgccactacaacgaggacat

SEQ ID NO: 502 - DNA, 50 bp, Artificial Sequence
  Description of Artificial Sequence/note = Synthetic Construct
tatttatgtttgatcctcagtgctttgtgtgacttgggctttgagaatta

SEQ ID NO: 503 - DNA, 50 bp, Artificial Sequence
  Description of Artificial Sequence/note = Synthetic Construct
gattggtttgacaatgaggactggctttgccaattaggttatatggcaga

SEQ ID NO: 504 - DNA, 50 bp, Artificial Sequence
  Description of Artificial Sequence/note = Synthetic Construct
ttaatncaccttttgtaagccctatacttactagtggcccaataccttct

SEQ ID NO: 505 - DNA, 50 bp, Artificial Sequence
  Description of Artificial Sequence/note = Synthetic Construct
acttagaaccagaccttcgaatcgctgtatcacaaagtgttaaaccaaga

SEQ ID NO: 506 - DNA, 50 bp, Artificial Sequence
  Description of Artificial Sequence/note = Synthetic Construct
atttatgttacctgcctggcccctgtaggcatttgagtttgcgacccctg

SEQ ID NO: 507 - DNA, 50 bp, Artificial Sequence; identical to SEQ ID NO: 506
  Description of Artificial Sequence/note = Synthetic Construct

SEQ ID NO: 508 - DNA, 50 bp, Artificial Sequence; identical to SEQ ID NO: 158
  Description of Artificial Sequence/note = Synthetic Construct

SEQ ID NO: 509 - DNA, 50 bp, Artificial Sequence
  Description of Artificial Sequence/note = Synthetic Construct
tgttaaaaaatgatccgctctgggtgtcgaatacgctaggtacgccactg

SEQ ID NO: 510 - DNA, 50 bp, Artificial Sequence
  Description of Artificial Sequence/note = Synthetic Construct
ccagtggtatgntttwgtagttgcctaaattgtaccttttgcagacgttt

SEQ ID NO: 511 - DNA, 50 bp, Artificial Sequence; identical to SEQ ID NO: 509
  Description of Artificial Sequence/note = Synthetic Construct

SEQ ID NO: 512 - DNA, 50 bp, Artificial Sequence
  Description of Artificial Sequence/note = Synthetic Construct
gttcttggaaactgcgactttaagcgaaacgacgtacagcaggtcctcga

SEQ ID NO: 513 - DNA, 50 bp, Artificial Sequence
  Description of Artificial Sequence/note = Synthetic Construct
attgccggcccatcaacagaacacccagacatgtgcaataataattaaat

SEQ ID NO: 514 - DNA, 50 bp, Artificial Sequence
  Description of Artificial Sequence/note = Synthetic Construct
gccagtcagatttcacggcantgccaatgtttctgtctgtacagcgntgt

SEQ ID NO: 515 - DNA, 50 bp, Artificial Sequence
  Description of Artificial Sequence/note = Synthetic Construct
ctcctgtgcttaccctgtatctgtaatctatatcaactatgccttcccca

SEQ ID NO: 516 - DNA, 50 bp, Artificial Sequence
  Description of Artificial Sequence/note = Synthetic Construct
tttatcaggggtttccgcttttgcttcttcctcattttcctcttgccgcc

SEQ ID NO: 517 - DNA, 50 bp, Artificial Sequence
  Description of Artificial Sequence/note = Synthetic Construct
gtgtccccacccaaatctcatcttgaattgtagttcccataatccccacg

SEQ ID NO: 518 - DNA, 50 bp, Artificial Sequence
  Description of Artificial Sequence/note = Synthetic Construct
tgttagttcacgcgagatctggttgtttaaaagagtntggcacctccccc

SEQ ID NO: 519 - DNA, 50 bp, Artificial Sequence
  Description of Artificial Sequence/note = Synthetic Construct
cttcctctctcgccatgtgatctctgcacacgccggctccccttcacctt

SEQ ID NO: 520 - DNA, 50 bp, Artificial Sequence
  Description of Artificial Sequence/note = Synthetic Construct
tcagtctgctccctatcttcggctgcccgtttagntgtggctcaagtggg

SEQ ID NO: 521 - DNA, 50 bp, Artificial Sequence
  Description of Artificial Sequence/note = Synthetic Construct
aaggtgcggcctggtttctccttgctgcttatagtaaaatgcgagaggaa

SEQ ID NO: 522 - DNA, 50 bp, Artificial Sequence
  Description of Artificial Sequence/note = Synthetic Construct
cctttcgcgtttcagttaacaaaccatttaaggaccatttgaggaaggaa

SEQ ID NO: 523 - DNA, 50 bp, Artificial Sequence
  Description of Artificial Sequence/note = Synthetic Construct
tgaaggcaggagaaattgccnaatcccncggaatagatgaaagaaatttc

SEQ ID NO: 524 - DNA, 50 bp, Artificial Sequence
  Description of Artificial Sequence/note = Synthetic Construct
tnatgtagactccttcgcaagactccatcagcgaaccatttgacactttt

SEQ ID NO: 525 - DNA, 50 bp, Artificial Sequence
  Description of Artificial Sequence/note = Synthetic Construct
acgctcttcccccagatatccacgtggctsgctccytcacctcmttcagg

SEQ ID NO: 526 - DNA, 50 bp, Artificial Sequence
  Description of Artificial Sequence/note = Synthetic Construct
cctgccactctgggttatmattgtctgtkngcangtctgtctcccccact

SEQ ID NO: 527 - DNA, 50 bp, Artificial Sequence
  Description of Artificial Sequence/note = Synthetic Construct
tttgtttgggacaccaagagcctggaactgcacrgcaccakctggtaaca

SEQ ID NO: 528 - DNA, 50 bp, Artificial Sequence
  Description of Artificial Sequence/note = Synthetic Construct
tggaccagtgctagtctgcaaactgtttgttaccagtccatgataagata

SEQ ID NO: 529 - DNA, 50 bp, Artificial Sequence
  Description of Artificial Sequence/note = Synthetic Construct
cccggtgctgaagttttagacggtatctctgaggggttatctaatctcaa

SEQ ID NO: 530 - DNA, 50 bp, Artificial Sequence
  Description of Artificial Sequence/note = Synthetic Construct
gaaaagtcgcccctggggaagctggttaactaggaccacccaagaccccc

SEQ ID NO: 531 - DNA, 50 bp, Artificial Sequence
  Description of Artificial Sequence/note = Synthetic Construct
aaaaaaggagcttgaacactcagaaccctgaaatatgtttaaccaatgga

SEQ ID NO: 532 - DNA, 50 bp, Artificial Sequence
  Description of Artificial Sequence/note = Synthetic Construct
catagcaggaataatggttactaacagaaaataacacatgggcctttcca

SEQ ID NO: 533 - DNA, 50 bp, Artificial Sequence
  Description of Artificial Sequence/note = Synthetic Construct
tcactctgtgtgtgtgtgtccgcgacctcgatctccttggccgtgagacc

SEQ ID NO: 534 - DNA, 50 bp, Artificial Sequence
  Description of Artificial Sequence/note = Synthetic Construct
acccactgcttcaaaacccaaaccctgattacagcncccctattcggcag

SEQ ID NO: 535 - DNA, 50 bp, Artificial Sequence
  Description of Artificial Sequence/note = Synthetic Construct
tnaataagacatggcacatttcagtcatccatcaaacatcaggggtgaat

SEQ ID NO: 536 - DNA, 50 bp, Artificial Sequence
  Description of Artificial Sequence/note = Synthetic Construct
gcttctgcgcagccgctctctcatcagatgatcgccatgatgatacaaca

SEQ ID NO: 537 - DNA, 50 bp, Artificial Sequence
  Description of Artificial Sequence/note = Synthetic Construct
gacaatggtctntccttcagntcgggntgaagaatgaccaaaggagaaat

SEQ ID NO: 538 - DNA, 50 bp, Artificial Sequence
  Description of Artificial Sequence/note = Synthetic Construct
atccttgtttcgntgtaagggattcagtggttggaaancagggagtggcc

SEQ ID NO: 539 - DNA, 50 bp, Artificial Sequence
  Description of Artificial Sequence/note = Synthetic Construct
gcgctcaaagggtgagttaactggatcgtatgccgggagcctattgtttt

SEQ ID NO: 540 - DNA, 50 bp, Artificial Sequence
  Description of Artificial Sequence/note = Synthetic Construct
ctcgcggtcctggccatccttgnaggcatgggcataacgttatgttgtgg

SEQ ID NO: 541 - DNA, 50 bp, Artificial Sequence
  Description of Artificial Sequence/note = Synthetic Construct
acncccangggattatctactcccctaaacagctatctctcttctaaagt

SEQ ID NO: 542 - DNA, 50 bp, Artificial Sequence
  Description of Artificial Sequence/note = Synthetic Construct
agccatggctatacgttatagacctgtatagttcttcccctcatacccta

SEQ ID NO: 543 - DNA, 50 bp, Artificial Sequence
  Description of Artificial Sequence/note = Synthetic Construct
gggcatatgaaatggactagctttgctaagggggatatctgggttggggg

SEQ ID NO: 544 - DNA, 50 bp, Artificial Sequence
  Description of Artificial Sequence/note = Synthetic Construct
cgggatcggtttggagtgctccgtctgcatcggatccgtctgtgtttgtg

SEQ ID NO: 545 - DNA, 50 bp, Artificial Sequence
  Description of Artificial Sequence/note = Synthetic Construct
ctttccctacccactgccactacnyctgactctggggccaaagcacatgc

SEQ ID NO: 546 - DNA, 50 bp, Artificial Sequence
  Description of Artificial Sequence/note = Synthetic Construct
acaccccaatgaactgacaccaagacccatttatacaaataagtttttcc

SEQ ID NO: 547 - DNA, 50 bp, Artificial Sequence
  Description of Artificial Sequence/note = Synthetic Construct
ctggagcagtcctccaaaatagacggggattagatcttataacggctgaa

SEQ ID NO: 548 - DNA, 50 bp, Artificial Sequence
  Description of Artificial Sequence/note = Synthetic Construct
ctcagtggcagatggtagaggtcaagagagganggacactagcaaccagg

SEQ ID NO: 549 - DNA, 50 bp, Artificial Sequence
  Description of Artificial Sequence/note = Synthetic Construct
tctttgctcccaggttayaatcctnaagcttgrcccaaataaactgtcta

SEQ ID NO: 550 - DNA, 50 bp, Artificial Sequence
  Description of Artificial Sequence/note = Synthetic Construct
agatgtggatactcaagatttctattggggaaaactgtggtccttagtaa

SEQ ID NO: 551 - DNA, 50 bp, Artificial Sequence
  Description of Artificial Sequence/note = Synthetic Construct
tgtattgctggcagcagtgaggtgggttaagggtgctatccggggctgca

SEQ ID NO: 552 - DNA, 50 bp, Artificial Sequence
  Description of Artificial Sequence/note = Synthetic Construct
ttaaaagtctcgcttccactgttcttcgtgtctctgagtccattctttgg

SEQ ID NO: 553 - DNA, 50 bp, Artificial Sequence
  Description of Artificial Sequence/note = Synthetic Construct
cattaaaagtctcactttcgctgttctccgggtctctgagtccattcttt

SEQ ID NO: 554 - DNA, 50 bp, Artificial Sequence
  Description of Artificial Sequence/note = Synthetic Construct
cccaccagaaggaagaaactccggacacatctgaacatctgaaggaacaa

SEQ ID NO: 555 - DNA, 50 bp, Artificial Sequence
  Description of Artificial Sequence/note = Synthetic Construct
agcacttttttccccccttaatttttaaacccatgtgtatttcaagggaa

SEQ ID NO: 556 - DNA, 50 bp, Artificial Sequence
  Description of Artificial Sequence/note = Synthetic Construct
tgcagttggtggcgacagagactgtagtgtggctggagtggtaggaaggg

SEQ ID NO: 557 - DNA, 50 bp, Artificial Sequence
  Description of Artificial Sequence/note = Synthetic Construct
aaagctttattgctcacacaaagcctgtttggtggtctcttcacacggac

SEQ ID NO: 558 - DNA, 50 bp, Artificial Sequence
  Description of Artificial Sequence/note = Synthetic Construct
acagccttgttgctcacacaaagcctgtttggtggtctcttcacacggac

SEQ ID NO: 559 - DNA, 50 bp, Artificial Sequence
  Description of Artificial Sequence/note = Synthetic Construct
gattaggcagcayacaggccacatcctcactcctgtgataacaagacaga

SEQ ID NO: 560 - DNA, 50 bp, Artificial Sequence
  Description of Artificial Sequence/note = Synthetic Construct
caggagaatagaaaattccaggcagcagtttcacatgactagcaaaagga

SEQ ID NO: 561 - DNA, 50 bp, Artificial Sequence
  Description of Artificial Sequence/note = Synthetic Construct
aagataaatagccagacaaccttggcaccaccaccyggccctaggagtta

SEQ ID NO: 562 - DNA, 50 bp, Artificial Sequence
  Description of Artificial Sequence/note = Synthetic Construct
acacctcactcttgttattttggcttctttctacaagcggcaagcagcyg

SEQ ID NO: 563 - DNA, 50 bp, Artificial Sequence
  Description of Artificial Sequence/note = Synthetic Construct
aacctgtattctcatggagagtcgtttgttactcaccaggygaatraacc

SEQ ID NO: 564 - DNA, 50 bp, Artificial Sequence
  Description of Artificial Sequence/note = Synthetic Construct
taaaagcttccctttaccctcccctcttcagatgcatctgtggcttgcca

SEQ ID NO: 565 - DNA, 50 bp, Artificial Sequence
  Description of Artificial Sequence/note = Synthetic Construct
tgaggccttcgttggaaacgggatttcttcatataatgctagacagaaga

SEQ ID NO: 566 - DNA, 50 bp, Artificial Sequence
  Description of Artificial Sequence/note = Synthetic Construct
ggttccagcattcattcgctccggttcccgcactcactcgcttgcatgct

SEQ ID NO: 567 - DNA, 50 bp, Artificial Sequence
  Description of Artificial Sequence/note = Synthetic Construct
tctcacaagcagagggagtttcagcatttcagcaagttgtttcttttctt

SEQ ID NO: 568 - DNA, 50 bp, Artificial Sequence
  Description of Artificial Sequence/note = Synthetic Construct
gatgttaagtctgctgggtctgagtgcactcaataaaagatcctcctgtt

SEQ ID NO: 569 - DNA, 50 bp, Artificial Sequence
  Description of Artificial Sequence/note = Synthetic Construct
tttcacaatgcatcccttcctaaaaactgaccaccatctctggactggtt

SEQ ID NO: 570 - DNA, 50 bp, Artificial Sequence
  Description of Artificial Sequence/note = Synthetic Construct
gtgaagggatatttgggagctcattgaggcctatggtgaaaaagaaaata

SEQ ID NO: 571 - DNA, 50 bp, Artificial Sequence
  Description of Artificial Sequence/note = Synthetic Construct
gttccagcactcatgcactccagttcccacctcgttcactcacatgctcc

SEQ ID NO: 572 - DNA, 50 bp, Artificial Sequence
  Description of Artificial Sequence/note = Synthetic Construct
tcctggtcacctccccataactggccttccccacacccttctttctttgt

SEQ ID NO: 573 - DNA, 50 bp, Artificial Sequence
  Description of Artificial Sequence/note = Synthetic Construct
actccctaaacacactgcgcgtgctcaattcccaagggtaaggagggcac

SEQ ID NO: 574 - DNA, 50 bp, Artificial Sequence
  Description of Artificial Sequence/note = Synthetic Construct
aattgtggcaggagtcttaacagcagtgggatgttgtattatcccttgtg

SEQ ID NO: 575 - DNA, 50 bp, Artificial Sequence
  Description of Artificial Sequence/note = Synthetic Construct
tttgcccaccctttcccgattgattctttctgaataatgccttttaacca

SEQ ID NO: 576 - DNA, 50 bp, Artificial Sequence
  Description of Artificial Sequence/note = Synthetic Construct
caccagaaggaagaaactccgaacacatccgaacatcagaaggaacaaac

SEQ ID NO: 577 - DNA, 50 bp, Artificial Sequence
  Description of Artificial Sequence/note = Synthetic Construct
cagtcggtgctgtctcacyyttgagcagccnygctctgactcagctgtca

SEQ ID NO: 578 - DNA, 50 bp, Artificial Sequence
  Description of Artificial Sequence/note = Synthetic Construct
cccttgttaaatcctccttggttgtggtcattggactgtcacctgccaag

SEQ ID NO: 579 - DNA, 50 bp, Artificial Sequence
  Description of Artificial Sequence/note = Synthetic Construct
gggacaagaactcagaccttgctaaactaaggagtaagaagactgcaaca

SEQ ID NO: 580 - DNA, 50 bp, Artificial Sequence
  Description of Artificial Sequence/note = Synthetic Construct
gtcaaagtgcttcattaaatgggtcctgttccctgtgccacccaactggg

SEQ ID NO: 581 - DNA, 50 bp, Artificial Sequence
  Description of Artificial Sequence/note = Synthetic Construct
tcattcacgtggattcaatgtagtactyggtgtatggcaaattcaagttt

SEQ ID NO: 582 - DNA, 50 bp, Artificial Sequence
  Description of Artificial Sequence/note = Synthetic Construct
ctataaaagcctcccccttgcattccctcggtggagctcccgaaccactt

SEQ ID NO: 583 - DNA, 50 bp, Artificial Sequence
  Description of Artificial Sequence/note = Synthetic Construct
tgtacaccctgtgatattattcgtaatatcctagggggatgttactccta

SEQ ID NO: 584 - DNA, 50 bp, Artificial Sequence
  Description of Artificial Sequence/note = Synthetic Construct
gggnaaatgantgatattcagtaatggtgctgggacatttggttttccat

SEQ ID NO: 585 - DNA, 50 bp, Artificial Sequence
  Description of Artificial Sequence/note = Synthetic Construct
cccctctagaggatgcagcatwcaaggygccatcttggaagcagagasca

SEQ ID NO: 586 - DNA, 50 bp, Artificial Sequence
  Description of Artificial Sequence/note = Synthetic Construct
tggctgaacactcccagtaacagtggctctgcgtttctcggaggtggagc

SEQ ID NO: 587 - DNA, 50 bp, Artificial Sequence
  Description of Artificial Sequence/note = Synthetic Construct
catccaaacaagctgcgatattctacccaacgatatagaagctgtagttg

SEQ ID NO: 588 - DNA, 50 bp, Artificial Sequence
  Description of Artificial Sequence/note = Synthetic Construct
gcccacccaacccatcacagcttccagcaacaccaacatggactgcttgg

SEQ ID NO: 589 - DNA, 50 bp, Artificial Sequence
  Description of Artificial Sequence/note = Synthetic Construct
tggaagaggattctaagcctcagatgagaacacagccctagccaacacct

SEQ ID NO: 590 - DNA, 50 bp, Artificial Sequence
  Description of Artificial Sequence/note = Synthetic Construct
ttcttccagaccctcccaatcctaaagagattaactaagatctgaatagg

SEQ ID NO: 591 - DNA, 50 bp, Artificial Sequence
  Description of Artificial Sequence/note = Synthetic Construct
cgtgacctcctaggaatgagccttcctagtgatgtgggacctaaacttct

SEQ ID NO: 592 - DNA, 50 bp, Artificial Sequence
  Description of Artificial Sequence/note = Synthetic Construct
tttaaatttggagccctcaaaatcatcttcggagaaaggcatagacctgt

SEQ ID NO: 593 - DNA, 50 bp, Artificial Sequence
  Description of Artificial Sequence/note = Synthetic Construct
aaaacaancacnangagccgggggnggggaatcagtatccagagttgcta

SEQ ID NO: 594 - DNA, 50 bp, Artificial Sequence
  Description of Artificial Sequence/note = Synthetic Construct
cacacagacagcagattagggctaacctggcaaggatacagcttgtctgc

SEQ ID NO: 595 - DNA, 50 bp, Artificial Sequence
  Description of Artificial Sequence/note = Synthetic Construct
tctctttgtcttgtgtctttatttattacaatctctcgtctccgcacacg

SEQ ID NO: 596 - DNA, 50 bp, Artificial Sequence
  Description of Artificial Sequence/note = Synthetic Construct
aaccacaacatnagaggacccancactcctcctaccaccaaaacaaaacc

SEQ ID NO: 597 - DNA, 50 bp, Artificial Sequence
  Description of Artificial Sequence/note = Synthetic Construct
agaggctcatagaaatggcacttactaaaacctcccttaactatcctcca

SEQ ID NO: 598 - DNA, 50 bp, Artificial Sequence
  Description of Artificial Sequence/note = Synthetic Construct
cngatcctcccctcnagttgagccttgagatgagactgcagtcctggctg

SEQ ID NO: 599 - DNA, 50 bp, Artificial Sequence
  Description of Artificial Sequence/note = Synthetic Construct
tttggacccccaaaattctactggcaggaagcaggctgagaaaactactc

SEQ ID NO: 600 - DNA, 50 bp, Artificial Sequence
  Description of Artificial Sequence/note = Synthetic Construct
cagaggctcataaaaacggcacttactaaaacctcccttaactatcctcc

SEQ ID NO: 601 - DNA, 50 bp, Artificial Sequence
  Description of Artificial Sequence/note = Synthetic Construct
ttccctcccttgtccaggtgtgcgctcaccattgctccatctgtgagggt

SEQ ID NO: 602 - DNA, 50 bp, Artificial Sequence
  Description of Artificial Sequence/note = Synthetic Construct
ctaaagacactttgtgctcagacctagaaatcttctcaattggctgccat

SEQ ID NO: 603 - DNA, 50 bp, Artificial Sequence
  Description of Artificial Sequence/note = Synthetic Construct
ctggaaggcctatgcacctaataatagaacctcatgtatcttccgctact

SEQ ID NO: 604 - DNA, 50 bp, Artificial Sequence
  Description of Artificial Sequence/note = Synthetic Construct
aattaaccaaggcttttaaaattccttggccaaaagctcttccattggtt

SEQ ID NO: 605 - DNA, 50 bp, Artificial Sequence
  Description of Artificial Sequence/note = Synthetic Construct
catttcccgtttgccccaagaatactcttgtctctaatcctaatgtaaca

SEQ ID NO: 606 - DNA, 50 bp, Artificial Sequence
  Description of Artificial Sequence/note = Synthetic Construct
cccaggtggtttggcatttgattagaatgattgggctgccccaggtgtgt

SEQ ID NO: 607 - DNA, 50 bp, Artificial Sequence
  Description of Artificial Sequence/note = Synthetic Construct
aggatctggtccagacaggataaagtgaagaaacnrgcaggaaccagcag

SEQ ID NO: 608 - DNA, 50 bp, Artificial Sequence
  Description of Artificial Sequence/note = Synthetic Construct
cacngctccacacctgrcttnnccttggcaggnntggatcnaggnccttg

SEQ ID NO: 609 - DNA, 50 bp, Artificial Sequence
  Description of Artificial Sequence/note = Synthetic Construct
tgctttgcaataaaagcttcttgcctttcgcttcattctgactcatccct

SEQ ID NO: 610 - DNA, 50 bp, Artificial Sequence
  Description of Artificial Sequence/note = Synthetic Construct
aggagcatcttttgttctaatatttggtctttgaccctagttcctgacac

SEQ ID NO: 611 - DNA, 50 bp, Artificial Sequence
  Description of Artificial Sequence/note = Synthetic Construct
ccaacctcaccctttgtgtccatgctccttaattttcttggttgtgagac

SEQ ID NO: 612 - DNA, 50 bp, Artificial Sequence
  Description of Artificial Sequence/note = Synthetic Construct
tctgtttgcgggagaagtttctgactttacctggagctgagtcaakttag

SEQ ID NO: 613 - DNA, 50 bp, Artificial Sequence
  Description of Artificial Sequence/note = Synthetic Construct
aatctcatgtcaaaaaaacactagctgaacacaagctaaggaacagagac

SEQ ID NO: 614 - DNA, 50 bp, Artificial Sequence
  Description of Artificial Sequence/note = Synthetic Construct
ttgacactcactttcggttttgtgtattggcttcgtgacaccaaacaggg

SEQ ID NO: 615 - DNA, 50 bp, Artificial Sequence
  Description of Artificial Sequence/note = Synthetic Construct
gggaggagaccacccctcatattgtcttatgcccaatttctgcctccaaa

SEQ ID NO: 616 - DNA, 50 bp, Artificial Sequence
  Description of Artificial Sequence/note = Synthetic Construct
caccagaaggaagaaactccggacacatctgaacatctgaaggaacaaac

SEQ ID NO: 617 - DNA, 50 bp, Artificial Sequence
  Description of Artificial Sequence/note = Synthetic Construct
cactcctgaagtcagcgagaccacgaacccaccgggaggaacaaacaact

SEQ ID NO: 618 - DNA, 50 bp, Artificial Sequence
  Description of Artificial Sequence/note = Synthetic Construct
gtaagagagaattcctcctgcctgactgcctttgaactgggacatcggtc

SEQ ID NO: 619 - DNA, 50 bp, Artificial Sequence
  Description of Artificial Sequence/note = Synthetic Construct
taacaacatgtttttgctgcagataatcagccagagcctgtttctctrct

SEQ ID NO: 620 - DNA, 50 bp, Artificial Sequence
  Description of Artificial Sequence/note = Synthetic Construct
gaagtgacagccttgtgtgtgatctttctgccctccccaagtttgcattt

SEQ ID NO: 621 - DNA, 50 bp, Artificial Sequence
  Description of Artificial Sequence/note = Synthetic Construct
tcttgctgctaaaactgcatacaacagccacccagccaagaggaattaat

SEQ ID NO: 622 - DNA, 50 bp, Artificial Sequence
  Description of Artificial Sequence/note = Synthetic Construct
cccagctgccatgctaaaagaagctcaggctagactattggatgatgaga

SEQ ID NO: 623 - DNA, 50 bp, Artificial Sequence
  Description of Artificial Sequence/note = Synthetic Construct
gctgagaaaacttttgcctgagtgctggtttcactttgcggcaccaagca

SEQ ID NO: 624 - DNA, 50 bp, Artificial Sequence
  Description of Artificial Sequence/note = Synthetic Construct
cagaaactcaaaagaatgcaaccatttgtctctcacctacctgtgacctg

SEQ ID NO: 625 - DNA, 50 bp, Artificial Sequence
  Description of Artificial Sequence/note = Synthetic Construct
ctctagtatagcatcacatgacagatagcaggccctgaaagaaatcaaag

SEQ ID NO: 626 - DNA, 50 bp, Artificial Sequence
  Description of Artificial Sequence/note = Synthetic Construct
cntctctctcctgccgccttgtgaagaaggtgcttgcttcccctttgcct

SEQ ID NO: 627 - DNA, 50 bp, Artificial Sequence
  Description of Artificial Sequence/note = Synthetic Construct
cctccgtatgctgagcgccggtcccctgggcccactgttctttctctata

SEQ ID NO: 628 - DNA, 50 bp, Artificial Sequence
  Description of Artificial Sequence/note = Synthetic Construct
ttgagtatcccttatccaaaatgcttgggaccagaagtgtttcggatttc

SEQ ID NO: 629 - DNA, 50 bp, Artificial Sequence
  Description of Artificial Sequence/note = Synthetic Construct
gtgactccacatgttaatggtcttattcaagctaagcagcatctactatc

SEQ ID NO: 630 - DNA, 50 bp, Artificial Sequence
  Description of Artificial Sequence/note = Synthetic Construct
cgttgcaacgtgcacagttcatgctaaggatccgtgcgatgcactctgat

SEQ ID NO: 631 - DNA, 50 bp, Artificial Sequence
  Description of Artificial Sequence/note = Synthetic Construct
ngtcnattgtttgactttcacacattcgacttccatacacgttttcagga

SEQ ID NO: 632 - DNA, 50 bp, Artificial Sequence
  Description of Artificial Sequence/note = Synthetic Construct
tactgaatcagaatctgcgttttaacaagatccccaggtgattcatatgc

SEQ ID NO: 633 - DNA, 50 bp, Artificial Sequence
  Description of Artificial Sequence/note = Synthetic Construct
ttggccanaaaacttttnttgaatcttctcattgggaaaattgggagatc

SEQ ID NO: 634 - DNA, 50 bp, Artificial Sequence
  Description of Artificial Sequence/note = Synthetic Construct
ttcacgtgcactgattggacaataaacaaatacgtaagtacctcttctct

SEQ ID NO: 635 - DNA, 50 bp, Artificial Sequence
  Description of Artificial Sequence/note = Synthetic Construct
acttagaaaatttcgaggaaggcactccaaagcacggggtcccctgaggc

SEQ ID NO: 636 - DNA, 50 bp, Artificial Sequence
  Description of Artificial Sequence/note = Synthetic Construct
acgcatcaccttgcattgcttcccatccttccctgcctcacttccctttt

SEQ ID NO: 637 - DNA, 50 bp, Artificial Sequence
  Description of Artificial Sequence/note = Synthetic Construct
cgaagccaaacgatcatacacaacatacaccacagtcataccctcaaggg

SEQ ID NO: 638 - DNA, 50 bp, Artificial Sequence
  Description of Artificial Sequence/note = Synthetic Construct
agtagcgctgtcatcaatccaacctagattagataagttaacaagcaaga

SEQ ID NO: 639 - DNA, 50 bp, Artificial Sequence
  Description of Artificial Sequence/note = Synthetic Construct
cgccatgattgtgaggcctccccagccatgtggaactgtgagtccattaa

SEQ ID NO: 640 - DNA, 50 bp, Artificial Sequence
  Description of Artificial Sequence/note = Synthetic Construct
atgattgtaagtttcctgaggcctccccagaagccgagcagatgccagca

SEQ ID NO: 641 - DNA, 50 bp, Artificial Sequence
  Description of Artificial Sequence/note = Synthetic Construct
atgcggcccctcgaccttggacttcccagcctccagaactgtaagaaata

SEQ ID NO: 642 - DNA, 50 bp, Artificial Sequence
  Description of Artificial Sequence/note = Synthetic Construct
gccgtctacgaaccagggaatgagccctcaccagaaactgaatctgccgg

SEQ ID NO: 643 - DNA, 50 bp, Artificial Sequence
  Description of Artificial Sequence/note = Synthetic Construct
gccatctacaagccaaggagagaggcctcagaagaaaccaaccctgccga

SEQ ID NO: 644 - DNA, 50 bp, Artificial Sequence
  Description of Artificial Sequence/note = Synthetic Construct
catggaacagattctccctcacagccctcagaaggaaccaaccctgccga

SEQ ID NO: 645 - DNA, 50 bp, Artificial Sequence
  Description of Artificial Sequence/note = Synthetic Construct
tagcccagtgagacccatttcggacttctgacctccagaactgtaagata

SEQ ID NO: 646 - DNA, 50 bp, Artificial Sequence
  Description of Artificial Sequence/note = Synthetic Construct
ttgtgagaccctgaagcagaggacccagctaagctgtgcccggactcctg

SEQ ID NO: 647 - DNA, 50 bp, Artificial Sequence
  Description of Artificial Sequence/note = Synthetic Construct
catcttgactgcaacctcatgagagaccctgagccagaaccacccagcta

SEQ ID NO: 648 - DNA, 50 bp, Artificial Sequence
  Description of Artificial Sequence/note = Synthetic Construct
gttcttcagttttgggactcggactggctctccttgctcctcagcttgca

SEQ ID NO: 649 - DNA, 50 bp, Artificial Sequence
  Description of Artificial Sequence/note = Synthetic Construct
tcacgtgagccaattcccctaataaatcycytctatccatcctattggtt

SEQ ID NO: 650 - DNA, 50 bp, Artificial Sequence
  Description of Artificial Sequence/note = Synthetic Construct
ccacaatcgcgtgagccaattccttaaaataaatctctctctacacacac

SEQ ID NO: 651 - DNA, 50 bp, Artificial Sequence
  Description of Artificial Sequence/note = Synthetic Construct
tctgcctgcctgatngtcttcgaactggaatatcagctctgcggattttg

SEQ ID NO: 652 - DNA, 50 bp, Artificial Sequence
  Description of Artificial Sequence/note = Synthetic Construct
taaaascaagctgtrccccgaccaccttgggcacatgtcgtcaggacctc

SEQ ID NO: 653 - DNA, 50 bp, Artificial Sequence
  Description of Artificial Sequence/note = Synthetic Construct
ctaaaatgtataaaascaagctgtrccccgaccaccttgggcacatgtkg

SEQ ID NO: 654 - DNA, 50 bp, Artificial Sequence
  Description of Artificial Sequence/note = Synthetic Construct
attgaagccctcaaaatcatctttggagaaaggcacagaccacagatgtt

SEQ ID NO: 655 - DNA, 50 bp, Artificial Sequence; identical to SEQ ID NO: 81
  Description of Artificial Sequence/note = Synthetic Construct

SEQ ID NO: 656 - DNA, 50 bp, Artificial Sequence
  Description of Artificial Sequence/note = Synthetic Construct
cacggtcctaccgatatgtgatgtcaccccyggaggcccagctgtaaaat

SEQ ID NO: 657 - DNA, 50 bp, Artificial Sequence
  Description of Artificial Sequence/note = Synthetic Construct
ccggatrcccagctttaaaatttctctcttttgtactctgtccctttatt

SEQ ID NO: 658 - DNA, 50 bp, Artificial Sequence
  Description of Artificial Sequence/note = Synthetic Construct
ggtctttgggtcttcatttctgaaggctcccatgtcacgtaaaactttga

SEQ ID NO: 659 - DNA, 50 bp, Artificial Sequence; identical to SEQ ID NO: 95
  Description of Artificial Sequence/note = Synthetic Construct

SEQ ID NO: 660 - DNA, 50 bp, Artificial Sequence; identical to SEQ ID NO: 96
  Description of Artificial Sequence/note = Synthetic Construct

SEQ ID NO: 661 - DNA, 50 bp, Artificial Sequence
  Description of Artificial Sequence/note = Synthetic Construct
tgcgggatggccaccttgcaggctgtaaccctttataagaaataaagtct

SEQ ID NO: 662 - DNA, 50 bp, Artificial Sequence
  Description of Artificial Sequence/note = Synthetic Construct
tgccttttctccwattaatctgccttttgtsagttgatttttcagtgaam

SEQ ID NO: 663 - DNA, 50 bp, Artificial Sequence; identical to SEQ ID NO: 128
  Description of Artificial Sequence/note = Synthetic Construct

SEQ ID NO: 664 - DNA, 50 bp, Artificial Sequence; identical to SEQ ID NO: 150
  Description of Artificial Sequence/note = Synthetic Construct

SEQ ID NO: 665 - DNA, 50 bp, Artificial Sequence
  Description of Artificial Sequence/note = Synthetic Construct
gccggaaactctaagagggtagaggwaaaatttttccttcyctnccatgg

SEQ ID NO: 666 - DNA, 50 bp, Artificial Sequence
  Description of Artificial Sequence/note = Synthetic Construct
tttacactgtggaatcaccctgaattctttcttgcatgagatccaagaac

SEQ ID NO: 667 - DNA, 50 bp, Artificial Sequence
  Description of Artificial Sequence/note = Synthetic Construct
tgctctaaaacttgcctcggtctctttttctgccttatgcccctcagtcg

SEQ ID NO: 668 - DNA, 50 bp, Artificial Sequence; identical to SEQ ID NO: 159
  Description of Artificial Sequence/note = Synthetic Construct

SEQ ID NO: 669 - DNA, 50 bp, Artificial Sequence
  Description of Artificial Sequence/note = Synthetic Construct
gtgtatgccccaaattgcaattctgttcttcacatgttattcccaaataa

SEQ ID NO: 670 - DNA, 50 bp, Artificial Sequence
  Description of Artificial Sequence/note = Synthetic Construct
agccgcttcaataaaagttgctgtctaataccaccarctcgcccttgaat

SEQ ID NO: 671 - DNA, 50 bp, Artificial Sequence; identical to SEQ ID NO: 670
  Description of Artificial Sequence/note = Synthetic Construct

SEQ ID NO: 672 - DNA, 50 bp, Artificial Sequence
  Description of Artificial Sequence/note = Synthetic Construct
attctccctttaaaacgcccagtcacctctgcacaaatcgaagctgagct

SEQ ID NO: 673 - DNA, 50 bp, Artificial Sequence
  Description of Artificial Sequence/note = Synthetic Construct
cctcattctccctttaaaacgcccagtcacctctgcacaaattggaatgg

SEQ ID NO: 674 - DNA, 50 bp, Artificial Sequence
  Description of Artificial Sequence/note = Synthetic Construct
tagcagattgcctgtgatgcgcatcacattctggtttaatgcttattcaa

SEQ ID NO: 675 - DNA, 50 bp, Artificial Sequence
  Description of Artificial Sequence/note = Synthetic Construct
cctgtgagtcctcctagcgaatcaccgaacctgggggtggtcttgggaac

SEQ ID NO: 676 - DNA, 50 bp, Artificial Sequence; identical to SEQ ID NO: 167
  Description of Artificial Sequence/note = Synthetic Construct

SEQ ID NO: 677 - DNA, 50 bp, Artificial Sequence; identical to SEQ ID NO: 170
  Description of Artificial Sequence/note = Synthetic Construct

SEQ ID NO: 678 - DNA, 50 bp, Artificial Sequence; identical to SEQ ID NO: 171
  Description of Artificial Sequence/note = Synthetic Construct

SEQ ID NO: 679 - DNA, 50 bp, Artificial Sequence
  Description of Artificial Sequence/note = Synthetic Construct
gctgcaaccctttatgagaaataaagctctcctttccaaatttatgaacc

SEQ ID NO: 680 - DNA, 50 bp, Artificial Sequence; identical to SEQ ID NO: 173
  Description of Artificial Sequence/note = Synthetic Construct

SEQ ID NO: 681 - DNA, 50 bp, Artificial Sequence
  Description of Artificial Sequence/note = Synthetic Construct
aagcatgattaatacaakytgctctgtgatgaacggatgccaaatagwcg

SEQ ID NO: 682 - DNA, 50 bp, Artificial Sequence; identical to SEQ ID NO: 176
  Description of Artificial Sequence/note = Synthetic Construct

SEQ ID NO: 683 - DNA, 50 bp, Artificial Sequence
  Description of Artificial Sequence/note = Synthetic Construct
cttctagcgaatcactgaacctgagggtggtcttggggacccccgacaca

SEQ ID NO: 684 - DNA, 50 bp, Artificial Sequence
  Description of Artificial Sequence/note = Synthetic Construct
gcgtcttgactgcgccgataccacgtgggacagagawgaactrcccagct

SEQ ID NO: 685 - DNA, 50 bp, Artificial Sequence; identical to SEQ ID NO: 181
  Description of Artificial Sequence/note = Synthetic Construct

SEQ ID NO: 686 - DNA, 50 bp, Artificial Sequence; identical to SEQ ID NO: 182
  Description of Artificial Sequence/note = Synthetic Construct

SEQ ID NO: 687 - DNA, 50 bp, Artificial Sequence
  Description of Artificial Sequence/note = Synthetic Construct
ataaacttgctcttctcactgtactccgcaactcgccttgaattccttcc

SEQ ID NO: 688 - DNA, 50 bp, Artificial Sequence
  Description of Artificial Sequence/note = Synthetic Construct
ctctgcttttgttgcttcattctttccttgctttgtttgtgcgttttgtc

SEQ ID NO: 689 - DNA, 50 bp, Artificial Sequence; identical to SEQ ID NO: 688
  Description of Artificial Sequence/note = Synthetic Construct

SEQ ID NO: 690 - DNA, 50 bp, Artificial Sequence; identical to SEQ ID NO: 196
  Description of Artificial Sequence/note = Synthetic Construct

SEQ ID NO: 691 - DNA, 50 bp, Artificial Sequence; identical to SEQ ID NO: 197
  Description of Artificial Sequence/note = Synthetic Construct

SEQ ID NO: 692 - DNA, 50 bp, Artificial Sequence
  Description of Artificial Sequence/note = Synthetic Construct
acctccaaccttctctttgttctttggacataccgaagaccacctggtct

SEQ ID NO: 693 - DNA, 50 bp, Artificial Sequence
  Description of Artificial Sequence/note = Synthetic Construct
acaactgtcttggtaaattatttttacctcccgcgccaccggccccagat

SEQ ID NO: 694 - DNA, 50 bp, Artificial Sequence; identical to SEQ ID NO: 204
  Description of Artificial Sequence/note = Synthetic Construct

SEQ ID NO: 695 - DNA, 50 bp, Artificial Sequence; identical to SEQ ID NO: 219
  Description of Artificial Sequence/note = Synthetic Construct

SEQ ID NO: 696 - DNA, 50 bp, Artificial Sequence; identical to SEQ ID NO: 220
  Description of Artificial Sequence/note = Synthetic Construct

SEQ ID NO: 697 - DNA, 50 bp, Artificial Sequence; identical to SEQ ID NO: 221
  Description of Artificial Sequence/note = Synthetic Construct

SEQ ID NO: 698 - DNA, 50 bp, Artificial Sequence; identical to SEQ ID NO: 222
  Description of Artificial Sequence/note = Synthetic Construct

SEQ ID NO: 699 - DNA, 50 bp, Artificial Sequence
  Description of Artificial Sequence/note = Synthetic Construct
agccatttgggtgtggtgtagaactggaaactgtgtcaagggtgactgag

SEQ ID NO: 700 - DNA, 50 bp, Artificial Sequence
  Description of Artificial Sequence/note = Synthetic Construct
aaattcccacttgcccatgctgtattcggagttgagcccaatctctctcc

SEQ ID NO: 701 - DNA, 50 bp, Artificial Sequence
  Description of Artificial Sequence/note = Synthetic Construct
tccccacttgtccttgctgtattcggagttgagcccaatctctctcccct

SEQ ID NO: 702 - DNA, 50 bp, Artificial Sequence; identical to SEQ ID NO: 226
  Description of Artificial Sequence/note = Synthetic Construct

SEQ ID NO: 703 - DNA, 50 bp, Artificial Sequence
  Description of Artificial Sequence/note = Synthetic Construct
ttgtactctgtccctttatttctcaagccagccgacgcttagggaaaata

SEQ ID NO: 704 - DNA, 50 bp, Artificial Sequence
  Description of Artificial Sequence/note = Synthetic Construct
actatcttgtgtgtgtctattatttctcaacctgccgatccgcctaggag

SEQ ID NO: 705 - DNA, 50 bp, Artificial Sequence
  Description of Artificial Sequence/note = Synthetic Construct
cgcccaataaattctgctcctcacccttcaatgtgtccgcgwgcctaatc

SEQ ID NO: 706 - DNA, 50 bp, Artificial Sequence
  Description of Artificial Sequence/note = Synthetic Construct
gkgacaagaacccgggttttagctgaactaaggagcaaaatyctgcawca

SEQ ID NO: 707 - DNA, 50 bp, Artificial Sequence
  Description of Artificial Sequence/note = Synthetic Construct
gttcctgaggtcggagcgttctccctattgcaatagtctttttgaataaa

SEQ ID NO: 708 - DNA, 50 bp, Artificial Sequence; identical to SEQ ID NO: 252
  Description of Artificial Sequence/note = Synthetic Construct

SEQ ID NO: 709 - DNA, 50 bp, Artificial Sequence; identical to SEQ ID NO: 253
  Description of Artificial Sequence/note = Synthetic Construct

SEQ ID NO: 710 - DNA, 50 bp, Artificial Sequence
  Description of Artificial Sequence/note = Synthetic Construct
cttcctcatncaccytataaaagcctttccttcaagcccctccggcggag

SEQ ID NO: 711 - DNA, 50 bp, Artificial Sequence; identical to SEQ ID NO: 255
  Description of Artificial Sequence/note = Synthetic Construct

SEQ ID NO: 712 - DNA, 50 bp, Artificial Sequence; identical to SEQ ID NO: 277
  Description of Artificial Sequence/note = Synthetic Construct

SEQ ID NO: 713 - DNA, 50 bp, Artificial Sequence; identical to SEQ ID NO: 278
  Description of Artificial Sequence/note = Synthetic Construct

SEQ ID NO: 714 - DNA, 50 bp, Artificial Sequence
  Description of Artificial Sequence/note = Synthetic Construct
tgtgggacttcaccttgtgatcgtgtgagtcaatactccttaataaactc

SEQ ID NO: 715 - DNA, 50 bp, Artificial Sequence; identical to SEQ ID NO: 301
  Description of Artificial Sequence/note = Synthetic Construct

SEQ ID NO: 716 - DNA, 50 bp, Artificial Sequence
  Description of Artificial Sequence/note = Synthetic Construct
gccacagaggtttccggccagaaaagcgacaccccaaggatcccatgaca

SEQ ID NO: 717 - DNA, 50 bp, Artificial Sequence
  Description of Artificial Sequence/note = Synthetic Construct
acactaaataaagctcttcttcgtcttcttcacccttcacttgtctgcgt

SEQ ID NO: 718 - DNA, 50 bp, Artificial Sequence
  Description of Artificial Sequence/note = Synthetic Construct
ttgargtctcccggttcgcgarctgtwctttctctyattgtatgcacaat

SEQ ID NO: 719 - DNA, 50 bp, Artificial Sequence; identical to SEQ ID NO: 323
  Description of Artificial Sequence/note = Synthetic Construct

SEQ ID NO: 720 - DNA, 50 bp, Artificial Sequence
  Description of Artificial Sequence/note = Synthetic Construct
agctacccctggacttttcagttacgtgaaccaataaattcccttttttg

SEQ ID NO: 721 - DNA, 50 bp, Artificial Sequence; identical to SEQ ID NO: 337
  Description of Artificial Sequence/note = Synthetic Construct

SEQ ID NO: 722 - DNA, 50 bp, Artificial Sequence
  Description of Artificial Sequence/note = Synthetic Construct
tttctgactcatccttgaattccttctcgcgatggtgtcaagagcctgga

SEQ ID NO: 723 - DNA, 50 bp, Artificial Sequence; identical to SEQ ID NO: 343
  Description of Artificial Sequence/note = Synthetic Construct

SEQ ID NO: 724 - DNA, 50 bp, Artificial Sequence
  Description of Artificial Sequence/note = Synthetic Construct
tgatttcagccttgtgagaccctgagcagaggacccagctaagccgtgcc

SEQ ID NO: 725 - DNA, 50 bp, Artificial Sequence
  Description of Artificial Sequence/note = Synthetic Construct
agccactgtacattttggggtttatttgttacagcagctagcgttacctt

SEQ ID NO: 726 - DNA, 50 bp, Artificial Sequence; identical to SEQ ID NO: 380
  Description of Artificial Sequence/note = Synthetic Construct

SEQ ID NO: 727 - DNA, 50 bp, Artificial Sequence
  Description of Artificial Sequence/note = Synthetic Construct
ttgatttcggccttgtgagaccctgagcagagaacccagccgagcccacc

SEQ ID NO: 728 - DNA, 50 bp, Artificial Sequence
  Description of Artificial Sequence/note = Synthetic Construct
tgcccaaattgcagattcgtgagcaaaataaatgattgttgttgttttaa

SEQ ID NO: 729 - DNA, 50 bp, Artificial Sequence
  Description of Artificial Sequence/note = Synthetic Construct
ctcagctttgcttgatcaacaggttttnttttctggtggtctttttgggg

SEQ ID NO: 730 - DNA, 50 bp, Artificial Sequence
  Description of Artificial Sequence/note = Synthetic Construct
tggtgctcycccttaccacagtaagcaataaactcagctttgtcttatca

SEQ ID NO: 731 - DNA, 50 bp, Artificial Sequence
  Description of Artificial Sequence/note = Synthetic Construct
gagagaccctgagccagaaccacccagctaagctgctcccgaattcctga

SEQ ID NO: 732 - DNA, 50 bp, Artificial Sequence
  Description of Artificial Sequence/note = Synthetic Construct
ggctgtgtctccctggtttgcaaactgttcactggaataaactctcctcc

SEQ ID NO: 733 - DNA, 50 bp, Artificial Sequence
  Description of Artificial Sequence/note = Synthetic Construct
ccctgctgtgccctgtccgaattcctgacccacagaatccgtgagcataa

SEQ ID NO: 734 - DNA, 50 bp, Artificial Sequence
  Description of Artificial Sequence/note = Synthetic Construct
agatgctcgcaccatgctttttgtccagccagcagaaytatgagccaaat

SEQ ID NO: 735 - DNA, 50 bp, Artificial Sequence
  Description of Artificial Sequence/note = Synthetic Construct
agccttcaagtcttcccagctgaggccccagacatcatggagcagagaca

SEQ ID NO: 736 - DNA, 50 bp, Artificial Sequence
  Description of Artificial Sequence/note = Synthetic Construct
tgcccttgaacttcccagcctgcagaaccatgagctaaataaacctcttt

SEQ ID NO: 737 - DNA, 50 bp, Artificial Sequence
  Description of Artificial Sequence/note = Synthetic Construct
gcctccagagggagcatggccctgctgacaccttkgatttcagcccagtg

SEQ ID NO: 738 - DNA, 50 bp, Artificial Sequence
  Description of Artificial Sequence/note = Synthetic Construct
gatgacgcagcaagaaggccctcaccagatgccggcnccwtgatcttgga

SEQ ID NO: 739 - DNA, 50 bp, Artificial Sequence
  Description of Artificial Sequence/note = Synthetic Construct
tctcgctttaataaattcctgctttcgctgcttcgttcctgtgtttcatt

SEQ ID NO: 740 - DNA, 50 bp, Artificial Sequence
  Description of Artificial Sequence/note = Synthetic Construct
tggtgtgagagcagaggaaaaacacggtttgagagagttttcccgaaaca

SEQ ID NO: 741 - DNA, 50 bp, Artificial Sequence
  Description of Artificial Sequence/note = Synthetic Construct
tctgtcttttgttacaggggtctattccaactaagaacttatgagggttg

SEQ ID NO: 742 - DNA, 50 bp, Artificial Sequence
  Description of Artificial Sequence/note = Synthetic Construct
tatctggatcgaccacattgaggaactgggaggaggcggagaactggaaa

SEQ ID NO: 743 - DNA, 50 bp, Artificial Sequence; identical to SEQ ID NO: 448
  Description of Artificial Sequence/note = Synthetic Construct

SEQ ID NO: 744 - DNA, 50 bp, Artificial Sequence
  Description of Artificial Sequence/note = Synthetic Construct
ctcattttcctcttgccgccgccatgtaagaagtgcctttcgcctcccgc

SEQ ID NO: 745 - DNA, 50 bp, Artificial Sequence
  Description of Artificial Sequence/note = Synthetic Construct
atgtgaagaaggacgtgtttgcttccccttccgccatgattgtaagtttc

SEQ ID NO: 746 - DNA, 50 bp, Artificial Sequence
  Description of Artificial Sequence/note = Synthetic Construct
atgattgnaagcttcctgaggcctcaccagaagccgagcagatgccggcg

SEQ ID NO: 747 - DNA, 50 bp, Artificial Sequence
  Description of Artificial Sequence/note = Synthetic Construct
gccatgcttcttgtacagcctgcagaaccgtgagccaaataaacctcttt

SEQ ID NO: 748 - DNA, 50 bp, Artificial Sequence
  Description of Artificial Sequence/note = Synthetic Construct
ctgtggagtgtactttcgcttcaataaatctgtgctttcgttactncgtt

SEQ ID NO: 749 - DNA, 50 bp, Artificial Sequence
  Description of Artificial Sequence/note = Synthetic Construct
tgggtggcaccacagttccgagaaatcttcacctttttccaggaatcttc

SEQ ID NO: 750 - DNA, 50 bp, Artificial Sequence; identical to SEQ ID NO: 564
  Description of Artificial Sequence/note = Synthetic Construct

SEQ ID NO: 751 - DNA, 50 bp, Artificial Sequence
  Description of Artificial Sequence/note = Synthetic Construct
tccttttacccctccctcaaagtgctttgctctcagcttctgccagaggc

SEQ ID NO: 752 - DNA, 50 bp, Artificial Sequence
  Description of Artificial Sequence/note = Synthetic Construct
ttgttacaggggtctgtcccagctaagaactatgaagggtagagagaaaa

SEQ ID NO: 753 - DNA, 50 bp, Artificial Sequence
  Description of Artificial Sequence/note = Synthetic Construct
gatatgccgcyggtaactcagggtaactcggatctcttccaccggtaaca

SEQ ID NO: 754 - DNA, 50 bp, Artificial Sequence; identical to SEQ ID NO: 582
  Description of Artificial Sequence/note = Synthetic Construct

SEQ ID NO: 755 - DNA, 50 bp, Artificial Sequence
  Description of Artificial Sequence/note = Synthetic Construct
catcttggaagcagagascaggccctcaccagacaccaaacctgctggna

SEQ ID NO: 756 - DNA, 50 bp, Artificial Sequence
  Description of Artificial Sequence/note = Synthetic Construct
cttgtgagaccctgagcagaggacccagctaagctgtgcccagactcctg

SEQ ID NO: 757 - DNA, 50 bp, Artificial Sequence
  Description of Artificial Sequence/note = Synthetic Construct
tcacgggccatggtcactcatatttggctcagaataaatctcttcaaata

SEQ ID NO: 758 - DNA, 50 bp, Artificial Sequence; identical to SEQ ID NO: 592
  Description of Artificial Sequence/note = Synthetic Construct

SEQ ID NO: 759 - DNA, 50 bp, Artificial Sequence
  Description of Artificial Sequence/note = Synthetic Construct
acaccttgattgcagccttgtgagagaccctgagccagaagacccaacta

SEQ ID NO: 760 - DNA, 50 bp, Artificial Sequence
  Description of Artificial Sequence/note = Synthetic Construct
cttctcagcctccataatcaagtgagccaattcccctaataaatcccttc

SEQ ID NO: 761 - DNA, 50 bp, Artificial Sequence
  Description of Artificial Sequence/note = Synthetic Construct
gagcagtaccgttcaataaaagattgctgtctaacaccactggctcaccc

SEQ ID NO: 762 - DNA, 50 bp, Artificial Sequence
  Description of Artificial Sequence/note = Synthetic Construct
ctcaggcaaagghaccachgghcacagaggtttctggccagaaaagbgac

SEQ ID NO: 763 - DNA, 50 bp, Artificial Sequence; identical to SEQ ID NO: 609
  Description of Artificial Sequence/note = Synthetic Construct

SEQ ID NO: 764 - DNA, 50 bp, Artificial Sequence
  Description of Artificial Sequence/note = Synthetic Construct
tgtgggatctgatgctaactccagggtagatagtgtcagaattgaattaa

SEQ ID NO: 765 - DNA, 50 bp, Artificial Sequence
  Description of Artificial Sequence/note = Synthetic Construct
cctgggtctccagcttgccaactcaccctgcagatcttgggacttctcag

SEQ ID NO: 766 - DNA, 50 bp, Artificial Sequence
  Description of Artificial Sequence/note = Synthetic Construct
taaatatgtgggtcaaactctgtttgtggctctcagctctgaaggctgtt

SEQ ID NO: 767 - DNA, 50 bp, Artificial Sequence
  Description of Artificial Sequence/note = Synthetic Construct
tacaccatgtggagcagaagaaccacccagctgagcccagccaacacaga

SEQ ID NO: 768 - DNA, 50 bp, Artificial Sequence
  Description of Artificial Sequence/note = Synthetic Construct
aaaaccaagctgtgctctgaccaccttgggcacatgtcgtcaggacctcc

SEQ ID NO: 769 - DNA, 50 bp, Artificial Sequence
  Description of Artificial Sequence/note = Synthetic Construct
tcanaggccatggtcactcatatttggctcagaataaatctcttcaaata

SEQ ID NO: 770 - DNA, 50 bp, Artificial Sequence
  Description of Artificial Sequence/note = Synthetic Construct
tgcttgcttcccctttgccttctgccatgattgtaagtttcctgaggcct

(19) United States
(12) Patent Application Publication
(10) Pub. No.: US 2006/0177825 A1
(43) Pub. Date: Aug. 10, 2006

(54) Global analysis of transposable elements as molecular markers of the developmental potential of stem cells

(76) Inventor: John F. McDonald, Arnoldsville, GA (US)

Correspondence Address: NEEDLE & ROSENBERG, P.C., SUITE 1000, 999 PEACHTREE STREET, ATLANTA, GA, 30309-3915, US

(21) Appl. No.: 10/554,759

(22) PCT Filed: Apr. 29, 2004
(86) PCT No.: PCT/US04/13667

§ 371 Date: Jan. 19, 2006

Related U.S. Application Data
(60) Provisional application No. 60/466,801, filed on Apr. 29, 2003.

(51) Int. Cl. C12Q 1/68; C12N 5/08; C12N 15/87

(52) U.S. Cl. 435/6; 435/366; 435/455

(57) ABSTRACT

This invention relates to the determination of expression patterns, DNA methylation patterns and chromatin properties of families of transposable elements in order to determine, classify and characterize the potential of stem cells to differentiate into germ layers including various types of somatic cell lineages.

This application claims priority to U.S. provisional application Ser. No. 60/466,801, filed Apr. 29, 2003, which is herein incorporated by this reference in its entirety.

### FIELD OF THE INVENTION

This invention relates to the determination of expression patterns, DNA methylation patterns and chromatin properties of families of transposable elements in order to determine, classify and characterize the potential of stem cells to differentiate into germ layers including various types of somatic cell lineages.

### BACKGROUND

The fertilized eggs (oocytes) of human and other multi-cellular animals have the potential to divide and give rise to progeny cells of the great variety of specialized cell types that comprise the fully developed organism. Cells that possess this full developmental potential are referred to as pluripotent (totipotent) stem cells. In addition to fertilized oocytes, cells isolated from primordial germ cells (PGCs) (e.g See Matsui et al. 1992 Derivation of pluripotential embryonic stem cells from murine primordial germ cells in culture. Cell 70: 841-847; Shamblott et al 1998 Derivation of pluripotent stem cells from cultured human-primordial germ cells Proc Natl Acad Sci., USA 95: 13726-13731), from early staged embryos (e.g. blastocists) (e.g, Evans and Kaufman 1981 Establishment in culture of pluripotential cells from mouse embryos Nature 292: 154-156.; Amit et al. 2000 Clonally derived human embryonic stem cell lines maintain pluripotency and proliferative potential for prolonged periods of culture. Dev Biol 227: 271-278) and from embryonic carcinomas (EC) (e.g Pierce 1967 Teratocarcinoma: a model for a developmental concept of cancer. Curr Topiccs Dev Biol 2: 223-246.), have also been shown to be pluripotent, i.e., to have the potential to divide and give rise to progeny cells of a great variety of specialized cell types. As embryos develop and their cells become determined to give rise to specialized cell types (e.g., neural cells, liver cells, etc.), they typically lose their pluripotency. The molecular genetic basis of pluripotency and the progressive loss of pluripotency as cells become determined to develop into specialized cell lineages, is a complex process associated with progressive changes in the chromatin status of chromosomes. The chromosomes of pluripotent stem cells are in a generally open configuration (euchromatin) due in part to the fact that most of the DNA comprising these chromosomes is hypomethylated (i.e., not methylated or displaying substantially reduced levels of methylation relative to differentiated cells) (Tada and Tada 2001 Toti-/pluripotential stem cells and epigenetic modifications Cell Struc and Func 26: 149-160). In contrast, the chromosomes of differentiated cells that have lost their pluripotency are typically condensed (heterochromatic) at numerous chromosomal locations due, in part, to the fact that the DNA comprising the condensed chromosomal regions are hypermethylated (Razin and Kafri 1994 DNA methylation from embryo to adult. Prog Nucleic Acid Res Mol Biol 48: 53-81). Gene sequences contained within heterochromatic, hypermethylated DNA are typically transcriptionally silent while genes contained within euchromatic, hypomethylated DNA may be transcriptionally active.

Recent studies have shown that when the nuclei (cellular organelle that contains chromosomes) isolated from even fully differentiated cells are transplanted into an unfertilized oocyte, the nuclei can become reprogrammed from the fully differentiated state to a fully pluripotent state. The molecular basis of this reprogramming is associated with hypomethylation of the DNA of the differentiated nuclei, a general opening of the chromatin structure and a general increase in gene transcription. Thus, the loss of pluripotency can be reacquired by factors contained in unfertilized oocytes.

The human genome comprises numerous families of transposable elements, such as retroelements, i.e., LIs (long interspersed nuclear elements), SINES (short interspersed nuclear elements) and LTR (long terminal repeat) elements, e.g. HERVs (human endogenous retroviruses) and DNA elements, i.e. Charlie- and Tigger groups (see Smit (1999) Interspersed repeats and other mementos of transposable elements in mammalian genomes. Current Opinion in Genetics & Development, 9: 657-663) that are widely distributed throughout the genome. To date, over 50 families of retroviral elements have been identified and the members of these families make up greater than 43% of the genome (See Li et al. (2001) Evolutionary analysis of the human genome. Nature, 409 (6822): 847-9). Each family can include hundreds to thousands of retroelements and the expression of these retroelement genes is known to be suppressed in differentiated cells due to hypermethylation (Yoder et al 1997 Cytosine methylation and the ecology of intragenomic parasites. Trends Genet 13: 335-340). In pluripotent stem cells retroelements are hypomethylated and the expression of retroelement genes is activated (Tada and Tada 2001). The present invention provides methods of determining patterns of transposable element expression and transposable element DNA methylation as well as methods for determining the chromatin status of transposable elements within the genome such that these patterns can be used as molecular markers of the developmental status of cells.

The present invention provides methods of determining patterns of transposable element expression, transposable element methylation and chromatin status of transposable elements within the genome such that these patterns can be used to classify and assess the developmental potential of a cell. All of the methods of the present invention can be utilized to analyze full-length transposable element sequences or fragments thereof. These transposable elements include retrolements and fragments thereof as well as DNA elements and fragments thereof from mammalian species. Thus, the present invention provides methods of determining patterns of retroelement expression, retroelement methylation and chromatin status of retroelements within the genome such that these patterns can be used to characterize the developmental potential of a cell. Also provided are methods of determining DNA element expression, DNA element methylation and chromatin state of DNA elements within the genome such that these patterns can be used to characterize the developmental potential of a cell.

### SUMMARY OF THE INVENTION

The present invention provides a method of determining an expression pattern of one or more families of transposable elements in a stem cell comprising determining expression of one or more families of transposable elements.

The present invention provides a method of assigning an expression pattern of transposable elements to the level of developmental potential of a cell comprising: a) determining expression of one or more families of transposable elements; and b) assigning the expression pattern obtained from step a) to the level of developmental potential of a cell.

Also provided by the present invention is a method of determining the developmental potential of a stem cell comprising: a) determining expression of one or more families of transposable elements in a stem cell to obtain an expression pattern;b) matching the expression pattern of step a) with a known expression pattern for a cell at different stages of developmental potential ranging from a fully pluripotent stem cell to a fully differentiated cell and; c) determining the developmental potential of the stem cell based on matching the expression pattern of a) with a known expression pattern for a cell at a specific developmental stage.

Further provided is a method of identifying a cellular differentiation induction factor comprising: a) determining expression of one or more families of transposable elements in a stem cell to obtain a first expression pattern; b) administering a putative induction factor to the cell; c) determining expression of one or more families of transposable elements in the cell after administration of the putative induction factor to obtain a second expression pattern; and d) comparing the second expression pattern with the first expression pattern such that if transposable elements are differentially expressed in the second expression pattern as compared to the first expression pattern, the induction factor is a cellular differentiation induction factor.

Also provided by the present invention is a method of identifying a factor that increases the developmental potential of a cell comprising: a) determining expression of one or more families of transposable elements in a cell to obtain a first expression pattern; b) administering a putative factor that increases developmental potential to the cell; c) determining expression of one or more families of transposable elements in the cell after administration of the putative factor to obtain a second expression pattern; and d) comparing the second expression pattern with the first expression pattern such that if transposable elements are differentially expressed in the second expression pattern as compared to the first expression pattern, the factor is effective in increasing the developmental potential of the cell.

Also provided by the present invention is a method of assigning a methylation pattern of transposable elements to the level of developmental potential of a cell comprising: a) determining methylation of one or more families of transposable elements; and b) assigning the methylation pattern obtained from step a) to the level of developmental potential of a cell.

Also provided by the present invention is a method of determining the developmental potential of a stem cell comprising: a) determining methylation of one or more families of transposable elements in a stem cell to obtain a methylation pattern; b) matching the methyation pattern of step a) with a known methylation pattern for a cell at different stages of developmental potential ranging from a fully pluripotent stem cell to a fully differentiated cell and; c) determining the developmental potential of the stem cell based on matching the methylation pattern of a) with a known methylation pattern for a cell at a specific developmental stage.

Further provided by the present invention is a method of identifying a cellular differentiation induction factor comprising: a) determining methylation of one or more families of transposable elements in a stem cell to obtain a first methylation pattern; b) administering a putative induction factor to the cell; c) determining methylation of one or more families of transposable elements in the cell after administration of the putative induction factor to obtain a second methylation pattern; and d) comparing the second methylation pattern with the first methylation pattern such that if there is a change in the second methylation pattern as compared to the first methylation pattern, the induction factor is a cellular differentiation induction factor.

Also provided is a method of identifying a factor that increases the developmental potential of a cell comprising: a) determining methylation of one or more families of transposable elements in a differentiated cell to obtain a first expression pattern; b) administering a putative factor that increases developmental potential to the cell; c) determining expression of one or more families of transposable elements in the cell after administration of the putative factor to obtain a second methylation pattern; and d) comparing the second methylation pattern with the first methylation pattern such that if there is a change in the second methylation pattern as compared to the first methylation pattern, the factor is effective in increasing the developmental potential of the cell.

Further provided is a method of assigning a chromatin status pattern of transposable elements to the level of developmental potential of a cell comprising: a) determining chromatin status of one or more families of transposable elements; and b) assigning the chromatin status pattern obtained from step a) to the level of developmental potential of a cell.

The present invention also provides a method of determining the developmental potential of a stem cell comprising: a) determining chromatin status of one or more families of transposable elements in a stem cell to obtain a chromatin status pattern; b) matching the chromatin status pattern of step a) with a known chromatin status pattern for a cell at different stages of developmental potential ranging from a fully pluripotent stem cell to a fully differentiated cell and; c) determining the developmental potential of the stem cell based on matching the chromatin status pattern of a) with a known chromatin status pattern for a cell at a specific developmental stage.

Also provided is a method of identifying a cellular differentiation induction factor comprising: a) determining chromatins status of one or more families of transposable elements in a stem cell to obtain a first chromatin status pattern; b) administering a putative induction factor to the cell; c) determining the chromatin status of one or more families of transposable elements in the cell after administration of the putative induction factor to obtain a second chromatin status pattern; and d) comparing the second chromatin status pattern with the first chromatin status pattern such that if there is a change in the second chromatin status pattern as compared to the first chromatin status pattern, the induction factor is a cellular differentiation induction factor.

Further provided is a method of identifying a factor that increases the developmental potential of a cell comprising: a) determining chromatin status of one or more families of transposable elements in a differentiated cell to obtain a first chromatin status pattern; b) administering a putative factor that increases developmental potential to the cell; c) determining expression of one or more families of transposable elements in the cell after administration of the putative factor to obtain a second chromatin status pattern; and d) comparing the second chromatin status pattern with the first chromatin status pattern such that if there is a change in the second chromatin status pattern as compared to the first chromatin status pattern, the factor is effective in increasing the developmental potential of the cell.

### DETAILED DESCRIPTION OF THE INVENTION

The present invention may be understood more readily by reference to the following detailed description of the preferred embodiments of the invention and the Examples included therein.

Before methods are disclosed and described, it is to be understood that this invention is not limited to specific methods, as such may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a nucleic acid” includes multiple copies of the nucleic acid and can also include more than one particular species of nucleic acid molecule. Similarly, reference to “a cell” includes one or more cells, including populations of cells.

Analysis of Expression Patterns

The present invention provides a method of determining an expression pattern of one or more families of transposable elements in a stem cell comprising determining expression of one or more families of transposable elements.

As used herein a “sample” can be of any type of stem cell from any organism and can be, but is not limited to, pluripotent stem cells derived from fertilized oocytes, from primordial germ cells (PGCs), from early staged embryos (e.g. blastocysts) and from embryonic carcinomas (EC). It is further contemplated that the biological sample of this invention can also be whole cells or cell organelles (e.g., nuclei). The cells may be part of a living tissue or growing in cell culture according to standard protocols widely available in the art.

As used here a “sample” can also be any determined and/or differentiated cell of a specialized type from any organism and can be, but is not limited to, differentiated brain or other neural cells, hepatic or liver cells, muscle cells, skin cells, connective tissue cells, etc. It is further contemplated that the biological sample of this invention can also be whole cells or cell organelles (e.g., nuclei). The cells may be part of a living tissue or growing in cell culture according to standard protocols widely available in the art.

The sample can be derived from a tissue or from an established cultured cell line. As utilized herein, the “cells” of the methods described herein can be derived from any animal. In a preferred embodiment, the organism of the present invention is a human. In addition, determination of expression patterns, methylation patterns and chromatin status is also contemplated for non-human animals which can include, but are not limited to, cats, dogs, birds, horses, cows, goats, sheep, pigs, guinea pigs, hamsters, gerbils, mice and rabbits.

The present invention also provides for the analysis of a sample comprising pluripotent stem cells or differentiated cells from a particular tissue or cell culture. The patterns obtained from differentiated cells can be compared to the expression patterns, methylation patterns and/or chromatin status patterns for pluripotent stem cells in order to access the differences between pluripotent cells and those that have lost their pluripotency, e.g. those that are differentiated.

The term “fully pluripotent” or “totipotent” when used herein refers to or describes the molecular or physiological status of a cell that is typically characterized by the potential to grow and differentiate into any specialized cell type. The term “pluripotency,” when used herein refers to or describes the molecular or physiological status of a cell that is typically characterized by the potential to grow and differentiate into specific cell subtypes, such as neural cells, muscle cells, hepatic cells, skin cells etc. Examples of fully pluripotent cells include but are not limited to fertilized oocytes, pluripotent stem cells isolated from primordial germ cells (PGCs), from early staged embryos (e.g. blastocists) and from embryonic carcinomas (EC).

There are numerous transposable element families that can be analyzed by the methods of the present invention, including, but not limited to, retroelement families and DNA element families. The retroelement families that can be analyzed utilizing the methods of this invention include but are not limited to, endogenous retroviruses (ERVs), short interspersed nuclear elements (SINEs), long interspersed nuclear elements (LINEs), the vertebrate long terminal repeat (LTR)-containing elements, and the poly(A) retrotransposons. The DNA element families that can be analyzed by the methods of the present invention include, but are not limited to the Mariner/Tci superfamily (e.g. human Mariner, Tigger, Marna, Golem, Zombi), hAT (hobo/Activator/Tam3) superfamily, TTAA superfamily (e.g. Looper), MITEs (e.g. MER85), MuDR superfamily (e.g. Ricksha), T2-family (e.g. Kanga 2) and others. Any combination of retroelement families and the members of these retroelement families can be analyzed by the methods of the present invention to determine a pattern of expression, a retroelement methylation pattern and/or a retroelement chromatin status pattern. For example, one of skill in the art could analyze the expression of ERVs as well as the expression of SIMEs or one of skill in the art could analyze the expression of SINEs, LINEs and ERVs. As stated above, any combination of families and members of transposable element families may be analyzed to provide an expression pattern, chromatin status pattern and/or a methylation pattern. Therefore, combinations of retroelement families and DNA element families can also be also analyzed by the methods of the present invention. A publicly available database, RepBase Update, contains consensus sequences of genomic repeats from different organisms that can be utilized to design the oligonucleotides utilized in the methods of the present invention. This database can be accessed at www.girinst.org. This database was utilized to identify consensus sequences for numerous retroelements which were then used to design oligonucleotide probes for the microarrays of the present invention.

Files were obtained from RepBase Update containing human-specific repeats (consensus sequences for transposon families). Selected RepBase files were then input into the OligoArray program, a publicly available software tool for microarray oligo-design at http://berry.engin.umich.edu/oligoarray and the design algorithm was run. The BLAST algorithm at http://www.ncbi.nlm.nih.gov/BLAST/(Altschul S F, Gish W, Miller W, Myers E W, Lipman D J Basic local alignment search tool. in J Mol Biol 1990 Oct 5;215(3):403-10)) was then utilized to verify compatibility of oligonucleotides in the OligoArray output file with transposon sequences in the human genome sequence (http://www.ncbi.nlh.nih.gov/genome/guide/human/). Selection of appropriate oligonucleotides was based on several criteria such as, the quality of match/specificity, technical parameters and the broad representation of transposable element families. Utilizing this approach, numerous oligonucleotides were designed based on these consensus sequences. The identifiers of retroelement consensus sequences and their corresponding oligonucleotide sequences which can utilized in the methods described herein, are listed in Table 1. Similar analyses can be performed to obtain consensus sequences for non-retroelement transposable element sequences.

TABLE 1

| | | |
|---|---|---|
| FLA | GAGTTCGAGACCAGCCTGGGCAACATAGCGAGACCCCGTCTCTAAAAAAA | SEQ ID NO: 1 |
| FLAM_A | GGAGTTCGAGACCAGCCTGGGCAACATAGCGAGACCCCGTCTCTAAAAAA | SEQ ID NO: 2 |
| FLAM_C | GGAGTTCGAGACCAGCCTGGGCAACATAGCGAGACCCCGTCTCTAAAAAA | SEQ ID NO: 3 |
| AluJo | GAGGCAGGAGGATCGCTTGAGCCCAGGAGTTCGAGGCTGCAGTGAGCTAT | SEQ ID NO: 4 |
| AluJb | GGAGTTCGAGACCAGCCTGGGCAACATGGTGAAACCCCGTCTCTACAAAA | SEQ ID NO: 5 |
| AluSc | TCACGAGGTCAAGAGATCGAGACCATCCTGGCCAACATGGTGAAACCCCG | SEQ ID NO: 6 |
| AluSg | CCAACATGGTGAAACCCCGTCTCTACTAAAAATACAAAAATTAGCCGGGC | SEQ ID NO: 7 |
| AluSp | CCAGCCTGACCAACATGGAGAAACCCCGTCTCTACTAAAAATACAAAAAT | SEQ ID NO: 8 |
| AluSq | CAACATGGTGAAACCCCGTCTCTACTAAAAATACAAAAATTAGCCGGGCG | SEQ ID NO: 9 |
| AluSx | CCAACATGGTGAAACCCCGTCTCTACTAAAAATACAAAAATTAGCCGGGC | SEQ ID NO: 10 |
| AluSz | CCAACATGGTGAAACCCCGTCTCTACTAAAAATACAAAAATTAGCCGGGC | SEQ ID NO: 11 |
| AluY | GAGATCGAGACCATCCTGGCTAACACGGTGAAACCCCGTCTCTACTAAAA | SEQ ID NO: 12 |
| AluYa5 | CGGGCGGATCACGAGGTCAGGAGATCGAGACCATCCCGGCTAAAACGGTG | SEQ ID NO: 13 |
| AluYa8 | GAAACCCCGTCTCTACTAAAACTACAAAAAATAGCCGGGCGTAGTGGCGG | SEQ ID NO: 14 |
| AluYb8 | AGACCATCCTGGCTAACAAGGTGAAACCCCGTCTCTACTAAAAATACAAA | SEQ ID NO: 15 |
| AluYb9 | AGACCATCCTGGCTAACAAGGTGAAACCCCGTCTCTACTAAAAATACAAA | SEQ ID NO: 16 |
| AluYc1 | GAGATCGAGACCATCCTGGCTAACACGGTGAAACCCCGTCTCTACTAAAA | SEQ ID NO: 17 |
| AluYc2 | GAGATCGAGACCATCCTGGCTAACAAGGTGAAACCCCGTCTCTACTAAAA | SEQ ID NO: 18 |
| AluYd3a1 | CGCCTGTAGTCCCAGCTACTCGGAGAGGCTGAGGCAGGAGAATGGCGTGA | SEQ ID NO: 19 |
| AluYe | ACCATCCTGGCTAACACGGTGAAACCCCGTCTCTACTAAAAATACAAAAA | SEQ ID NO: 20 |
| LTR26B | ATGGATTTGAGGTTTCCTCCCATCTCCTCATTCGGCGGCCCTACGATTAA | SEQ ID NO: 21 |
| LTR26C | ACGGATTTGAGGTTTCCTCCCATCTCCTCATTCGGCAGCCCTACGATTAA | SEQ ID NO: 22 |
| LTR26D | GGCGTATTGACTTGCTGTGTGCATCGGGCAATGAACCTATTACGGTTACA | SEQ ID NO: 23 |
| AluYa1 | GAGATCGAGACCATCCTGGCTAACACGGTGAAACCCCGTCTCTACTAAAA | SEQ ID NO: 24 |
| AIuYa4 | CGGGCGGATCACGAGGTCAGGAGATCGAGACCATCCCGGCTAAAACGGTG | SEQ ID NO: 25 |
| AluYb3a1 | GAGATCGAGACCATCCTGGCTAACACGGTGAAACCCCGTCTCTACTAAAA | SEQ ID NO: 26 |
| AluYb3a2 | GAGATCGAGACCATCCTGGCTAACACGGTGAAACCCCGTCTCTACTAAAA | SEQ ID NO: 27 |
| AluYe5 | ACCATCCTGGCTAACACGGTGAAACCCCGTCTCTACTAAAAATACAAAAA | SEQ ID NO: 28 |
| AluYf1 | GAGATCGAGACCATCCTGGCTAACACGGTGAAACCCCGTCTCTACTAAAA | SEQ ID NO: 29 |
| AluYg6 | GAGATCGAGACCATCCTGGCTAACACGGTGAAACCCCGTCTCTACTAAAA | SEQ ID NO: 30 |
| AluYh9 | GAGATCGAGACCATCCTGGCTAACGCGGTGAAACCCCGCCTCTACTAAAA | SEQ ID NO: 31 |
| AluYl6 | AGATCGAGACCATCCTGGCTAACACGGTGAAACCCCGTCTCTACTAAAAA | SEQ ID NO: 32 |
| AluYbc3a | AGATCGAGACCATCCTGGCTAACACGGTGAAACCCCGTCTCTACTAAAAA | SEQ ID NO: 33 |
| AluYe2 | GACCATCCTGGCTAACACGGTGAAACCCCGTCTCTACTAAAAATACAAAA | SEQ ID NO: 34 |
| AluYf2 | GATCGAGACCATCCTGGCTAACACAGTGAAACCCCGTCTCTACTAAAAAA | SEQ ID NO: 35 |
| ALU | GAGGCAGGAGGATCGCTTGAGCCCAGGAGTTCGAGGCTGCAGTGAGCTAT | SEQ ID NO: 36 |
| MIR | GGCTCTGCCACTTACTAGCTGTGTGACCTTGGGCAAGTTACTTAACCTCT | SEQ ID NO: 37 |
| L1PA2 | ATCACATGGACACAGGAAGGGGAATATCACACTCTGGGGACTGTGGTGGG | SEQ ID NO: 38 |
| L1PA7 | CCTGTCGGGGGGTGGGGGGCTAGGGGAGGGATAGCATTAGGAGAAATACC | SEQ ID NO: 39 |
| L1PA11 | TGGGCTTAATACCTAGGTGATGGGATGATCTGTGCAGCAAACCACCATGG | SEQ ID NO: 40 |
| L1PA15 | TCGGGTACTATGCTTATTACCTGGGTGACGAAATAATCTGTACACCAAAC | SEQ ID NO: 41 |
| L1PB1 | ATCTCAGAAATCACCACTAAAGAACTTATTCATGTAACCAAACACCACCT | SEQ ID NO: 42 |
| L1PB3 | AAGTGGGAGCTAAGCTATGGGTACGCAAAGGCATACAGAGTGGTATAATG | SEQ ID NO: 43 |
| L1MA2 | GGGAAGGGTAGTGGGGGGTTGGTGGGGAGGTGGGGATGGTTAATGGGTAC | SEQ ID NO: 44 |
| L1MA5 | ATAGGGAGAGGTTGGTTAATGGATACAAAATTACAGCTAGATAGGAGGAA | SEQ ID NO: 45 |
| L1MA9 | AGATCTTAAGTGTTCTCACCACACAAAAAAAAATGGTAACTATGTGAGGT | SEQ ID NO: 46 |
| THE1B | CTGCACAWGCTCTCTTGCCTGCCGCCATGTAAGACGTGMCTTTGCTCCTC | SEQ ID NO: 47 |
| MSTA | TCCCCTTGGTGCTGTCCTCGTGATAGTGAGTGAGTTCTCGTGAGATCTGG | SEQ ID NO: 48 |
| MSTC | GATTAATGGATTAATGGGTTATCATGGGAGTGGGACTGGTGGCTTTATAA | SEQ ID NO: 49 |
| MLT1A | TGAGGACACAGTGAGAAGGCGCCGTCTACGAACCAGGGAATGAGCCCTCA | SEQ ID NO: 50 |
| MLT1B | GGAGAAGACGGCCATCTACAAGCCAAGGAGAGAGGCCTCAGAAGAAACCA | SEQ ID NO: 51 |
| MLT1C | CCAGCAAACCACCAGAAGCTAGGGGAGAGGCATGGAACAGATTCTCCCTC | SEQ ID NO: 52 |
| MLT1D | GGTCAGAGTCAGAGAAGGAGATGTGACGACGGAAGCAGAGGTCGGAGTGA | SEQ ID NO: 53 |
| MLT1E | GATTCCGTCTTGNCGNCANTCTTGCTGAGAGNCTCTCTTGCTGGCTTTGA | SEQ ID NO: 54 |
| MLT1F | TGTAGTCCCCTCCCACATTGAATAGGGCTGACCTGTGTGACCAATAGAAT | SEQ ID NO: 55 |
| THE1BR | CAAGAGGTGACTTGGGTGCTGTTAAAGGCATTCAGTTTTAAAAGGGAAGC | SEQ ID NO: 56 |
| MSTAR | TCTTTTTGATTTTACAGGCTCATAGGTGGAAGGAACTTGCCTTGTCTCAG | SEQ ID NO: 57 |
| MLT1R | AGCCTGATCATGTAACAGAAANNNCAATAGCGTTCTCTGGAAAGAANACC | SEQ ID NO: 58 |
| MLT2A1 | GGGTGTTGCCAAAGGAGGTTAACATTGGACTCAGTGGGCTGGGGAGAGGC | SEQ ID NO: 59 |
| MLT2B2 | TTCCAGATGAGATTAGCATTTGAATCAGCGGACTGAGTAAAGAAGATTGC | SEQ ID NO: 60 |
| MLT2C2 | CTCAAGACTGCAACGTGGAAATCCTGCTGNTTTWCCAGCCTCCAAGCCTT | SEQ ID NO: 61 |
| MLT2D | GGCTAGGCTATGGTGTCCAGACGTTTGGTCAAACATTAGTCTGGGTGTTT | SEQ ID NO: 62 |
| LTR2 | CAATGCTCCCAGCTGATTAAAGCCTCTTCCTTCATAGAACCGGTGTCTAA | SEQ ID NO: 63 |
| LTR3 | GCAAGGAGCCCCCTGACCCCTTCTTCCAAACATACTCTTTTGTCTTTGTC | SEQ ID NO: 64 |
| LTR4 | ATCCTCCTGTCCCACCCATTGGTCTCTCCTGTCCCTTGATTCCTGCAACA | SEQ ID NO: 65 |
| LTR5 | ACTCAGAGGCTGGTGGGATCCTCCATATGCTGAACGTTGGTTCCCCGGGC | SEQ ID NO: 66 |
| LTR11 | AACTCCGTCACTGTAATCCCAATGTAAAGCAAGAATTCCAAACCAGGAAA | SEQ ID NO: 67 |
| LTR12 | GCTTCATTCTTGAAGTCAGCGAGACCAAGAACCCACCGGAAGGAACCAAT | SEQ ID NO: 68 |
| LTR13 | CTTGTGTCTTTATTTCTACACTCTCTCGTCTCCGCACACGGGGAGAAAAA | SEQ ID NO: 69 |
| MER1A | AAGCTTCATCTGTAKTTACAGCCGCTCCCCATCACTCGCATTACCGCCTG | SEQ ID NO: 70 |
| MER1B | TGATCTGAGGTGGAACAGTTTCATCCCGAAACCATCCCCGCCCCCCGGTC | SEQ ID NO: 71 |
| MER2 | AAAATCCACGGATGCTCAAGTCCCTGATATAAAATGGCGTAGTATTTGCA | SEQ ID NO: 72 |
| MER3 | ATGTGGCTAYTGAGCACTTGAAATGTGGYTAGTGCGACTGAGGAACTGAA | SEQ ID NO: 73 |
| MER4A | GGACCTCAAGATCTTTACCCTAAAACAGTTCTGYTGAVYTTCACCTTGGC | SEQ ID NO: 74 |
| MER4B | TTGGTCTCCGCAACCCCTTATNTCATAACCCGGACATTCCTTTCCATTGA | SEQ ID NO: 75 |
| MER4C | CCTCCCTCTTTCCCCTCCAGCCCGCTTTTCCCCTTTAAATATTGAAGCCC | SEQ ID NO: 76 |
| MER5A | GTCCCCGGACCAGCAGCATCAGCATCACCTGGGAACTTGTTAGAAATGCA | SEQ ID NO: 77 |
| MER5B | TCAGTATTTTTTAAARCTCYYCAGGTGATTCCAATGTGCAGCCAAGGTTG | SEQ ID NO: 78 |
| MER6 | AAGTCGCAGTTTCCAAGAACCTATCGACGACGTTAAGTGAGGACTTACTG | SEQ ID NO: 79 |
| MER8 | AAAAATCCGCGTATAAGTGGACCCACGCAGTTCAAACCCGTGTTGTTCAA | SEQ ID NO: 80 |
| MER9 | GCTGTGAGACCCCTGATTTCCCACTTCACACCTCTATATTTCTGTGTGTG | SEQ ID NO: 81 |
| MER11A | TGATTTTGCCCTTGTCCTGTTTCCTCAGAAGCATGTGATCTTTGTTCTCC | SEQ ID NO: 82 |
| MER11B | ACTTGCTGGTTTTTGCGGCTTGTGGGGCATCACGGAACCTACCGACATGT | SEQ ID NO: 83 |
| MER20 | CCCCACAACAAAGAATTATCCGGCCCAAAATGTCGATAGTGCCAAGGTTG | SEQ ID NO: 84 |
| MER21 | SAGCAGAGGTAAAACATGGTTTGAGAGAGGTTTTYCTGMAAYAGRAGGGC | SEQ ID NO: 85 |
| MER21B | CGGTCAGAAGCACAGGTNACAACCTGGNGCTTGCGACTGGCATCTGAAGT | SEQ ID NO: 86 |
| MER22 | TGAGTCTCCCCAAAAGTGGAGCCCTTGTGATGACGAGCACAGGTCCGCCT | SEQ ID NO: 87 |
| MER28 | AAGACGANGAGGATGAAGACCTTTATGATGATCCACTTCCACTTAATGAA | SEQ ID NO: 88 |
| MER30 | TTTTAAGAAAGTTTACGAATTTGTGTTGGGCCGCATTCAAAGCCATCCTG | SEQ ID NO: 89 |
| MER35 | GATGAAAAGGGGATCCTGTGCAGAAACCACACTACCCATCAGAGAAGCAA | SEQ ID NO: 90 |
| MER39 | GGCAGGTCATAGAAACTAGAACTCCTCTCCCCCAAAGCAAGCCATAAAAC | SEQ ID NO: 91 |
| MER44A | AGGGTTCGGTACTATCCGCGGTTTCAGGCATCCACTGGGGGTCTTGGAAC | SEQ ID NO: 92 |
| MER44C | CGCACCTCAAACTGCAAAAGTTACGGCCACAGTGCGTGATAAGTGCTTAG | SEQ ID NO: 93 |
| MER45 | GAAATTCTTAATAATTTTTGAACAAGGGGCCCCGCATTTTCATTTTGCAC | SEQ ID NO: 94 |
| MER48 | TGTTGTTGTGGACGCGCTCTCGGGGTTSGAACCGAYACAAGARCCTTACA | SEQ ID NO: 95 |
| LOR1 | TCTTCCTTGGCAATAMTYRTTGTCTCAGTGATTGGCTTTCTGTGCAGTGA | SEQ ID NO: 96 |
| SVA | GGGGAAAGGTGGGGAAAAGATTGAGAAATCGGATGGTTGCCGTGTCTGTG | SEQ ID NO: 97 |
| ALR | GTGGAGATTTCAGCCGCTTTGAGGTCAATGGTAGAATAGGAAATATCTTC | SEQ ID NO: 98 |
| MSR1 | GGAGTCAAGACCCCCCAGCCCCTCCTCCCTCAGACTCATGAGTCCAGACC | SEQ ID NO: 99 |
| TAR1 | ACTCATGGAGGGTTAGGGTTCAGGTTCGGGTTCGGGTTCGGGTTCGGGTT | SEQ ID NO: 100 |
| CER | GGTTCTGAGTGTTTGTCCCTCACATAGGATTCCAGAACACTGCTGCTGGG | SEQ ID NO: 101 |
| BSR | TCACAATGCCCCTGTAGGCAGAGCCTAGACAAGAGTTACATCACCTGGGT | SEQ ID NO: 102 |
| HSATII | GGGTCCATTCGATGATGATCACACTGGATTTCATTCCATAATTCTATTCG | SEQ ID NO: 103 |
| HSATI | CCACTGTCTGTGCTGTGTCTTTCAAAGGTCAGAAGAGATTGNACCTTTGT | SEQ ID NO: 104 |
| R66 | TGCRTTTACAAACCTTTAGCTAGACACAGAGCGCTGATTGGTGCGTTTTT | SEQ ID NO: 105 |
| SN5 | CCTGACTCCTGAGTCACGTTACTGTCCCACTATACGTTAAGAGGAGGGAA | SEQ ID NO: 106 |
| HIR | AATATCAGGAACACCGGCATGTGCACTTAGGACCATGTTTTAATTTTTCA | SEQ ID NO: 107 |
| GGAAT | GGAATGGAATGGAATGGAATGGAATGGAATGGAATGGAATGGAATGGAAT | SEQ ID NO: 108 |
| KER | GGATGAGGCAGGAAAGACAGCTGAGGGTCAGAACCCAGGCAGGTCCAATG | SEQ ID NO: 109 |
| TIGGER1 | ACTCGCTGAAGGCTCAGATGATCGTTAGCATTTTTTAGCAATAAAGTATT | SEQ ID NO: 110 |
| TIGGER2 | TAAAGTTACACCGAGTGTGCCTGCCTCTCCTGCCTCCCCTTCCACCTCCT | SEQ ID NO: 111 |
| GSAT | GGGACTCAGGAGGATGTTGAGGGAGACAGAGGGGTGAAGCGTTGAGACGA | SEQ ID NO: 112 |
| GSATX | CAGGCGGCCAGNCTTTCAGGGGGAGGATGAAGTAGGCCTGGGACAAAAGC | SEQ ID NO: 113 |
| HERVL | AGGACTCTACTTCTAATAGTATGGAGAACACTGATAGTCCTTGGCATGAA | SEQ ID NO: 114 |
| HERVK | CCCTGTCACTTGGGTTAAGACCATTGGAAGTACATCGATTATAAATCTCA | SEQ ID NO: 115 |
| HERVR | AACCCAACAGTATCAGGTGCTCAGAACCGATGAAGAAGCTCAAGATTGAG | SEQ ID NO: 116 |
| HRES1 | TGGTTAATGTGTAACAAGGAGGCAGTAGGCCCCAGGTGTCCAGCCAGAGG | SEQ ID NO: 117 |
| HERVE | AAAAGTGAGGACGAGAGTAAGAACTCCCACTAAAAGTGAAAATTCTCAAA | SEQ ID NO: 118 |
| HERVH | CATACCACCCCCCAAAAATTTTCACTGCCCCAACACTTCAACACTATTTT | SEQ ID NO: 119 |
| HERVI | TTGTAGGATGCTGTGTCATACCCTGTGCCCTAGGATTAATACAAAAGCTC | SEQ ID NO: 120 |
| LTR14 | GCCTCCACTCTTTATGAACTCTTAACCTGTCTCTTCTCATTCCTTTGTCA | SEQ ID NO: 121 |
| HERVKC4 | CCGGATCATTCACAGAGTTCAATTCAATTAACAGTTTAAGCCCCCAAAAA | SEQ ID NO: 122 |
| MER4I | AGAGATCAGACGAAACCTGAGACCAGAGACTCATTTTCTTCTAAAATGCT | SEQ ID NO: 123 |
| MER49 | ACATGCATGTTTGTTCAATACGCATGCGTCAGGACCACCTTCATGAATAT | SEQ ID NO: 124 |
| MER4D | CAACCCCCCTTATCTTAACTCAAGCTGACTTCAACTCTTCAGGCAGAGCT | SEQ ID NO: 125 |
| MER39B | GCCCTCCTGTCTCTCAGTCCCATTCTCCCCCGAGGCTAGCCATAGAAACT | SEQ ID NO: 126 |
| IN25 | TCTTGGAGAAGGGATCCTTGTTCCCCNCTGGCNCTGGTANNCCACTGCAG | SEQ ID NO: 127 |
| MER61 | AAGCCTAAWTTTTCGTGGCCGTGTGACAAGGACCCCGTCTTTAGCTGAAC | SEQ ID NO: 128 |
| HERV3 | CAACCCTTGCCAAATGAAGAGAACTGCCTTCNCATGAAGAATTAANTAGT | SEQ ID NO: 129 |
| HERV9 | GCACAGAGCCATACAACTAATACCCCTACTTATAGGGTTAGGAATGGCTA | SEQ ID NO: 130 |
| HERVS71 | AAACTGGACTAATGTCCTTGTCCCAACAGGTAGATGCTGATTTAAATAAC | SEQ ID NO: 131 |
| HSMAR1 | CACTTCTTCAAGCATCTCGACAACTTTTTGCAGGGAAAACGCTTCCACAA | SEQ ID NO: 132 |
| HSMAR2 | TGGTATCATCGCTTACAAAAGTGTCTTGAACTTGATGGAGCTTATGTTGA | SEQ ID NO: 133 |
| L1 | AAACAACCCCATCAAAAAGTGGGCAAAGGATATGAACAGACACTTCTCAA | SEQ ID NO: 134 |
| L1MA10 | GTGATGGTTTCACGGGTGTATGCATATGTCCAAACTCATCAAATTGTATA | SEQ ID NO: 135 |
| L1MB3 | TCAGTTTGGGAAGATGAAAAAGTTCTGGAGATGGATGGTGGTGATGGTTG | SEQ ID NO: 136 |
| L1MB7 | AGATAGTGGTGATGGTTGCACAACTCTGTGAATATACTAAAAACCACTGA | SEQ ID NO: 137 |
| L1MC2 | ATGTTAATAATAGGGGAAACTGTGTGNGGGNGGGGTGAGGGGGTATATGG | SEQ ID NO: 138 |
| L1MC3 | CTGTTGGAGTGGGAGGTTACAGATAAGCAAGGGGAGGAGGCTAGAATGAT | SEQ ID NO: 139 |
| L1MC4 | TATTTAGGGGTAANGGGGCATCATGTCTGCAACTTACTCTCAAATGGTTC | SEQ ID NO: 140 |
| L1MD1 | GCAGGAGGGAAGTGGGTGTGGCTATAAAAGGGCAACATGAGGGATCCTTG | SEQ ID NO: 141 |
| L1MD2 | GNGNGGGGGAAGGGAGGTGGGTGTGGCTATAAAAGGGCAGCACGAGGGAT | SEQ ID NO: 142 |
| L1ME2 | AGTGGTTGCCTCTGGGGAGGGTGANTGACTGGAAAGGGGCATGAGGGAAC | SEQ ID NO: 143 |
| L1ME3A | GGCAAAACTAATCTATGSTGTTAGAAGTCAGGATAGTGGTTACCCTTGGG | SEQ ID NO: 144 |
| LSAU | GGTGTTGGGAGAGCCTCAGCCGGAATTTCGTGGACGGACAAGGGCACAGA | SEQ ID NO: 145 |
| LTR1 | CTAGAGGTTTGAGCAGCGGGGCACTGAAGAAGCGAGCCACACCCCCATCG | SEQ ID NO: 146 |
| LTR15 | ATCCTCCTCAACCCCATCGGTCTCTCTGATTCCTAAATCATCCCCAAACA | SEQ ID NO: 147 |
| LTR8 | TTTCTCTATTGCAATTCCCCTGTCTTGATGAATCGGCTCTGTCTAGGCAG | SEQ ID NO: 148 |
| LTR9 | TAAACTCCTCGTGTGTGTCCGTGTCCTAAATTTTCCTGGCGCGNGACGAC | SEQ ID NO: 149 |
| MER31 | CCTGTACCTATCGCAATGGTCCTGAATAAAGTCTGCCTTACCGTGCTTTA | SEQ ID NO: 150 |
| MER34 | GCCCAAACCCCTTTGTCTTGTCACGTTTTCACAATTTACTACTCTTTGTC | SEQ ID NO: 151 |
| MER41A | GCAACGTCAGGAAGTTACCCTATATGGTCTAAAAAGGGGAGGCATGAATA | SEQ ID NO: 152 |
| MER41B | TGCCATGGCAACGTCAGGAAGTTACCCTATATGGTCTAAAAAGGGGAGGA | SEQ ID NO: 153 |
| MER41C | TAGCAGAGCACATCTCCCCCGTAATGTTCTTTGGCTTTGTTATCCTATAT | SEQ ID NO: 154 |
| MER50 | TGGCCCTCTTCCAAGTGTACTTCGCTTCCTTTCGTTCCTGCTCTAAAACT | SEQ ID NO: 155 |
| MER63A | TTCAAGCTACCAACGTGATGTCACTGAATGSGGAGTTGGGAAAAGATATA | SEQ ID NO: 156 |
| MER63B | ATGTCACTGAATGSGGAGTTGGGAAGAGATGCACAGTAGCACACYATTAT | SEQ ID NO: 157 |
| MER63C | ACAATGTAACGGCTACAGACACGACACACTTTTAAGTTTAATCTGCATTA | SEQ ID NO: 158 |
| MER65A | GAATATGCACATAGTTTACTATGGCACGCGTATTCCCATTGCAATGCTCT | SEQ ID NO: 159 |
| MER65B | ACATTTGCCTGACAACTGTCTCACRAACCTAGCTACTGCAAGAGCCTACT | SEQ ID NO: 160 |
| MER66A | AGACTAGCTGAAACAGGGCCAGGGCAAAAGCACCTCTCCATAAGACACAC | SEQ ID NO: 161 |
| MER66B | CTTGAACACCAGACCAAATTGAAGACTAGCTGAAACAGGGCCAGGGCAAA | SEQ ID NO: 162 |
| MER67A | GCCTCAACCTCGGCCTATAAAGACTTGAACAAACACTAACATAGTTTCTA | SEQ ID NO: 163 |
| MER67B | CACAGAACAACTCCATCCAAACCCCTGCACTAAGAGACTTGACCAAACTC | SEQ ID NO: 164 |
| MER67C | TCTTGAGAACATGTATGTAATGGGCTGTATCTGCTCGGCTATATAAAAGG | SEQ ID NO: 165 |
| MER68A | AACCCTGGGCACTGAGTCTCTAATGAGCTTCCCTGGTAGACAACATTTCA | SEQ ID NO: 166 |
| MER68B | TTCCCTTTGCTGATCTTGCCGTGTATCCTTACNRTGTCGCTGTAATAAAT | SEQ ID NO: 167 |
| MER69A | CCCCCAAATTGTATAAGCTTCAGGCCCCACAAAACCTGGATCTGCCCCTG | SEQ ID NO: 168 |
| MER69B | TTACAAAATCATTGTCATATGAAGAGGCGATCAAAGAGTATGCAGCCAAA | SEQ ID NO: 169 |
| MER70A | TGTTCTGTCTCACCGGACTCAGACAAGTTGGTAACCAGTGCACAGTGAAC | SEQ ID NO: 170 |
| MER70B | TCNGACCCCTATTCCTGGTGGTTGGCATAGTGATGATCTTTGCTATTCTC | SEQ ID NO: 171 |
| MER72 | GGCATGAAGCTCAATTGCACATGTGCATGTTTCTCCTTTCATAAATATTC | SEQ ID NO: 172 |
| MER73 | GGTGACGGGGTACGACTGGGTTTCAAACAACTTATGTCAGGCCTAAAAAT | SEQ ID NO: 173 |
| MER74 | GGGGGTATGGGCTCTGGATTGGTTGGTTTGCATATGAAAGGCGCGCTCCC | SEQ ID NO: 174 |
| MER75 | TGGCCGAAGATTCATTTGATGAATCCGATTTTTCCGAAATAGACGATTCT | SEQ ID NO: 175 |
| MER76 | TGTTGCCTTAATCGGCTNCTCTGACACCCGGCAGCTCAGCTCTCTCTCCA | SEQ ID NO: 176 |
| MER77 | GGTGAGCTTCCCTGGTTGGCAATACTCTNTGCATGTTGTCACACATCGTT | SEQ ID NO: 177 |
| MER80 | CCATAGGCTTCACCAGACTGCCAAAGGGGCCCATGGCACAAAAAAGGTTA | SEQ ID NO: 178 |
| MER82 | NTGCAAATGACCGNGAAAGTGCTNCAAGTATTGATTTTGGGGTTACAAAT | SEQ ID NO: 179 |
| MLT1G | CACAAATTCTTTGACACTCTTCCCATCGAGGAGTGGGGTCCGTNTCCTCT | SEQ ID NO: 180 |
| PABL_A | AATAAAAACTCTCTTCCTCCCCAGTTCATCTGCATCTCGTTATTGGGCCA | SEQ ID NO: 181 |
| PABL_B | CCAGTTCATCTGCATCTCGTTATTGGGCCACGAGAATAAGCAGCCCGACC | SEQ ID NO: 182 |
| MER57I | GCAGTTATGGGGGATACTCGGCTCTTTGCACATTTGGATNAGAGAAGCAT | SEQ ID NO: 183 |
| MER65I | CCTGGATAAATTCCCCTGGGGAACTTGAGGCCCCATATACACGAAATTAC | SEQ ID NO: 184 |
| MER41I | TTTGTTGGGAACTCAGTTACAAATAACCCTCACCATACCAGTACTTTCTG | SEQ ID NO: 185 |
| PTR5 | CATGCTTAAGGAGCCCTTCAGCCTGCCACTGCACTGTGGGAACACTGGCC | SEQ ID NO: 188 |
| LIM2_5 | CGCCTCCTCCACAAAGAAGAACCAAAATAGCGAGTAGATAATCACACTTT | SEQ ID NO: 187 |
| LTR10A | TGCTCCATCTGCGAGACGCACCCTTCTATAGAAGTAAAATTGCCTTGCTG | SEQ ID NO: 188 |
| LTR10B | GCTGAGAGACCCTTTGTCCTTTGGCTCAGTGTTGGTTCTTCTTTGCAGCA | SEQ ID NO: 189 |
| LTR10C | CAGTGTACTCTCATGGCAAAACTGCTGGTGAGTGTACCCTTTCTGCAGAA | SEQ ID NO: 190 |
| LTR16A | CTGCATTGCAGCCCAACTTCTCCCTCTGCCCAATCCTGCTTCCTTCCCTT | SEQ ID NO: 191 |
| LTR17 | CCAAGAACCCCAGGTCAGAGAACACGAGGCTTGCCACCATCTTGGAAGTG | SEQ ID NO: 192 |
| MER41D | GCACGTAGGCACAGCTTAGTTTAGTCTTTACATAGACAAGACTCCTATAT | SEQ ID NO: 193 |
| MER51A | TCCGCAACCAATCAGACGTTTGCATAGGAGTGTAACTTTGTAACTTCACT | SEQ ID NO: 194 |
| MER51B | CTTTACTTCGTCCTCTTCATTTACATAGGGCGTACCCCAAGTAACCAATG | SEQ ID NO: 195 |
| MER57A | ATCTTCTACCACATGGCTGCACTGGAGTCTCTGAACCTACTCTGGTTCTG | SEQ ID NO: 198 |
| MER57B | TATAAATTTGTTCCGACCACGAGGCATCCCTGGAGTCTCTCTGAATCTGC | SEQ ID NO: 197 |
| MER65C | CAACCCTGGCTGCTGAAACTGCCTGTTGTAACCTGAAACCAGTTTTATCT | SEQ ID NO: 198 |
| MER83 | TCTGCAGCCCAAGAACCATCCTATAAAATCTCCAGCAAGCCTTTGTCTCC | SEQ ID NO: 199 |
| MER84 | CATAAATGCTCCTAAGGAAAAATCCACCGCGGCGCGCTCAGTCCTCTCTT | SEQ ID NO: 200 |
| HERV16 | TTGACTATGATGTGTAGGAGGGGTAGGGCTGCTTTAGTAAAATGAGTAAG | SEQ ID NO: 201 |
| HERV17 | GAAGGCACCCCTCCCGAGGAAATCTCAACTGCACGACCCCTACTACGCCC | SEQ ID NO: 202 |
| PMER1 | GTTCTCAACCTTCCTAATGCCGCGGCCCTTTAATACAGTTCCTGTGGGTC | SEQ ID NO: 203 |
| MER54 | TGAAAGATACACTGTAAACACCCACAACCAMCTTCCCTGGAGCCCCATCA | SEQ ID NO: 204 |
| LTR18A | TGTACATACGGCTTGCGCCCAGGCTCACTCGCGCCCAGAGAGAGAGTAAA | SEQ ID NO: 205 |
| LTR18B | ATGAGAGAGCTGCTGAATAAAACCATATTTCACCTGCCTACGGCCCCCCG | SEQ ID NO: 206 |
| LTR19A | AGAGAGTGCTCCTGACTGAAATCGGCCAGAAGCCCCTCTCAGGTTTATTC | SEQ ID NO: 207 |
| LTR19B | GACTGKWGAGCCGCTTTTCGTGTTTCTTTCCTCTTTCTTTAATTCTTACA | SEQ ID NO: 208 |
| LTR20 | AATAAATTCTGCTCYACCTCACCCTTCAATGTGTCTGCATGCCTAATTCT | SEQ ID NO: 209 |
| LTR16C | GTAACTNGCTTGATAACGCACCCTTTATTGGCTTCCTTCCCTTCCCTGTC | SEQ ID NO: 210 |
| LTR21A | CTGCTTYCCTTGACTGTKAWGGGGGCAGCCGRCAGGTTAATAAARGCTTG | SEQ ID NO: 211 |
| LTR21B | CAATAAAGCTTGCTTGCCTGACTTTGGGTCTCYTCATCCTTTCTCTCGGC | SEQ ID NO: 212 |
| MER85 | TTGAGCAGTAGGATATAAATAACTCCCACATGCTTAGCGTTCCAATAATG | SEQ ID NO: 213 |
| LTR22 | GTGCYAGCTGNTTAGGGCCAGCWGCWGTKACAAACCTYYCTTGGWGTSTG | SEQ ID NO: 214 |
| LTR23 | CCTTTAAAAACCACTTGTAACTGCTGCTAATTGGAGTGTATATTCAGGGC | SEQ ID NO: 215 |
| LTR24 | AAACCTTAACTTCTCCACTTTGGAACGCTGACCCCATTCCTTTGGAGTCT | SEQ ID NO: 216 |
| HERV23 | GTCCTGTCCCCCCAACCATGTGAGATAGAGCCATCTGGGAATGAGCTTTA | SEQ ID NO: 217 |
| HERV18 | AGCGGGAATATTAGTGGTGAGTTGTTGCTCCCTGTATTGTTGCTGTGGCC | SEQ ID NO: 218 |
| MER87 | ACTTACTGGCTGTCGWGCGGTGAGCAGTACCAGCTTTGGATTCAGTTACA | SEQ ID NO: 219 |
| MER74A | AATGGCAGTCGTCTCCTGATCTGTTGGCCTTACCATACCTGAATAATAAT | SEQ ID NO: 220 |
| MER74B | CTTTTCAATGGCAGTCGTCTCCTGATCTGTTGGCCTTACCATACCTSAAT | SEQ ID NO: 221 |
| MER88 | AGGGGAACTTGTGGCAGGGACCAGCCTTATCACACTGGTGCACCTGGTCA | SEQ ID NO: 222 |
| MER54B | GAGCCCAGTCTGCTAGGCGGGAGAGATGCCTCTAAGTTCTTATCTCTGGC | SEQ ID NO: 223 |
| MER31A | GGCTCCTGAACCTTCTCCTAGGCCCATCTGTGCACTTCCTTGTAAAATCC | SEQ ID NO: 224 |
| MER31B | GCCCTGTCCTTGGCCTGCWTAGCCCAGTTTTAGCAAGAATCCTGCTAAGT | SEQ ID NO: 225 |
| MER67D | ATCCACCTGCCTTTTGTTTCAGNGGAGTTGAGTTCAANCTCTAACCCCTA | SEQ ID NO: 226 |
| MER31I | GATGATTCAGCTGGTCCTTAATGAACAAAAGGCMACCCAACAAGAAAATG | SEQ ID NO: 227 |
| CHARLIE1 | TTCCACATTGCAACTAACCTTTAAGAAACTACCACTTGTCGAGTTTTGGT | SEQ ID NO: 228 |
| CHARLIE1A | CACCGCAACTAACCTTTAAGAAACTACCACTTGTTGAGTTTTGGTGTAGT | SEQ ID NO: 229 |
| CHARLIE1B | CAGTGGAGTTTTCCAGAGGCTACATGACGTGTGATGTCGCAACAGATTGA | SEQ ID NO: 230 |
| CHARLIE2 | TAAAATTCTGTGGGGGAAGTGGAATGGAAATACGAGTTCAAGGAGAAAAA | SEQ ID NO: 231 |
| MER30B | CAATCTTTTGGCTTCCCTGGGCCACATTGGAAGAAGAATTGTCTTGGGCC | SEQ ID NO: 232 |
| MER45B | CCGCATACGAGTTAAATGCTCTTATATTTGCATTTAAAACTGGCATTGCA | SEQ ID NO: 233 |
| MER45C | GCGAGTATCCCCGTGCCCGAGGGAGCGTGACATTAAATAGCAAATAAAAA | SEQ ID NO: 234 |
| LTR25 | CTCTCCGCTGRCAGAGAGCTTTCTTCTTTCACTTATTAAACTTTCACTCC | SEQ ID NO: 235 |
| LTR26 | TCTCAGTGTAATTGGTCTGTTACTGCGCAGTGGGCATATGAACCTGTTGG | SEQ ID NO: 236 |
| HERVK9I | ATCCCGACTCCTGCGAGAAGTAGCTCACCGTGACAAAGCTGCCTTTGCTT | SEQ ID NO: 237 |
| HERVH48I | TCTCTCAAGAATACCCCAAAAATTAAGTTTTTCTTTTTCCAAGGTGCCCA | SEQ ID NO: 238 |
| MER11C | CCTGTGATCTCGCCCTGCCTCCACTTGCCTTGTGATATTCTATTACCYTG | SEQ ID NO: 239 |
| MER11D | TTCATCCCCATGTGACCATCTCACCTCATAATCAAATGACCCTAAATCCC | SEQ ID NO: 240 |
| LTR10D | GGCGACTGGCCAAGGAGAAGCACCCCTCTGCGCAGAAGTAAAATTGCTTT | SEQ ID NO: 241 |
| LTR14A | CCACACTCGCGATGGCCCCCTGGTCCCACTTTCTCTCTCAAACTGTCTTT | SEQ ID NO: 242 |
| LTR14B | TTTGCAGCCTCCATACTTAGCGTTGGCCCCCTGGACCCACTTTCTCTCTC | SEQ ID NO: 243 |
| LTR27 | GTGGGACAAGAACTTGGGAATCAGTGCACAAGCCAGACTTGGCCTGGGAA | SEQ ID NO: 244 |
| LTR28 | ATTGATCCCCACCCTTCACCTATTTTACATATACCCACCCTTTCCTAATT | SEQ ID NO: 245 |
| LTR29 | TTAATCAATCTGCCTTNTGTCAGTGATTTTTCAGCGAACCTTCAGGGGGC | SEQ ID NO: 246 |
| LTR30 | CTTTTTTTCTCTCTTGGTCCGATCCGTGTCTCTCWCTCGCCGCGGGCWGC | SEQ ID NO: 247 |
| LTR31 | TTTCTCTTTTGCAAAACCCATCGTCACAGTGATTGRCTTACTGCGCGCGG | SEQ ID NO: 248 |
| MER61B | ACCCTTTCCTGACTGATTCTCTCTGAATAATGCCCACCTGCGCACTGGGA | SEQ ID NO: 249 |
| MER61C | CCGACCCGCCCCACAAGTGTTTACATCAGATGCTTTTGTGCAGATGAGGG | SEQ ID NO: 250 |
| MER92A | CGCTTGCCCACTGTCYCCTTTCTACTGGTTCTGCTTAYCYCTCCCTATAA | SEQ ID NO: 251 |
| MER92B | TTCTGCCTGAACTTTGAGATGCTTGCAGATCTTATGGTCAGAGCGTTCTC | SEQ ID NO: 252 |
| MER92C | TATCTACCCCTTCCTATAAAAGTCCAAGGCAAAACCACCCTGCCGAGACA | SEQ ID NO: 253 |
| MER93 | GCCCTGGGTTCCTACGTAAGCAAACCGAAACCTAACTCAGNCGTTTCTTA | SEQ ID NO: 254 |
| MLT1H | CACAGATGCATGAGGGAGCCCAGCCGAGACCAGAAGAACCACCCAGCTGA | SEQ ID NO: 255 |
| LIP_MA2 | GAACCCAGAAACAAATCCATACATYTACAGCGAACTCATTTTCGACAAAG | SEQ ID NO: 256 |
| LTR32 | ATGTAAGTCCCCAATAAACCCTATGTCTCATTTGCTGGCTCTGGGTCTCT | SEQ ID NO: 257 |
| GOLEM | GCACAACGACGAAATCGCCTAACGACGCATTTCTCAGAACGTATCCCCGT | SEQ ID NO: 258 |
| ZOMBI | TAGTGACACCTTTGCTTTCTGATGGTTCAATGTACACAAACTTTGTTTCA | SEQ ID NO: 259 |
| ZOMBI_A | CGGATTTTCAGATTTGGGATGCTCAACCGGTAAGTATAATGCAAATATTC | SEQ ID NO: 260 |
| ZOMBI_B | NCTGCCAGNCAACNACAGNTTGTGCACCTNGNTGGCARAGANACTGACAC | SEQ ID NO: 261 |
| LTR33 | CGCTGTTGCTAGCCCCGGGGTGCTTCACCATCCCTTGTTGGTTTCCCTTA | SEQ ID NO: 262 |
| L1PA12_5 | AAGTCAGCTTCAAATAAAGACCCTGCACAAAGCCTCGGCCCGGTGAAAAC | SEQ ID NO: 263 |
| L1PA16_5 | GACAGCCANACAATAGACAGCCTGTCAATAGANATAGCCACACAATAATA | SEQ ID NO: 264 |
| L1PBA_5 | AAGAATCTGAACAGCAGCCCTTGAGTCCCAGATCTTCCCTCTGACATAGT | SEQ ID NO: 265 |
| L1PBB_5 | AATCTACCCACCTGCTTTAGCCACARCTGGTKYYTACCCAKGGAYACCTC | SEQ ID NO: 266 |
| L1M3A_5 | AAGAAACATAWTCACATTCAARGGAGTCCCAATATGGCTATCAGCAGATT | SEQ ID NO: 267 |
| L1M3B_5 | AGTGGMAATCTCATCAGCCCAGGGATCTRACAGGAGAAGGTCTTCCTCCC | SEQ ID NO: 268 |
| L1M3C_5 | YACATCMATAGAAAAGGTCTGAGAGAGYCCCAGAATCCCTAGCCAGGCTG | SEQ ID NO: 269 |
| L1M3D_5 | GTCGCGCTACGCTGATANGATTNANCATACCCTANATGCTCGGCGACTGC | SEQ ID NO: 270 |
| L1MB6_5 | CACTCAGTGCGAAMAGCATTATACCTGGGGGCATTTGTTGAAAACAWTTA | SEQ ID NO: 271 |
| L1MCA_5 | TGAAAGTGGACTTGGATTAGTTGTAAATGTATATTGCAAACTCTAGGGCA | SEQ ID NO: 272 |
| L1MCB_5 | CTGACACCTACAGCTACAGCAAACAGTAAACACAGTCTAACTCTTAGCCA | SEQ ID NO: 273 |
| L1MEA_5 | ACCACAGCCACTGGAAAGAGTGGGGAAAATCCCGGAAAGGAGAGAGCCAG | SEQ ID NO: 274 |
| L1MEC_5 | ACAAAAATATCCAGCACCCAACAAGGTAAAATTCACAATGTCTGGCATCC | SEQ ID NO: 275 |
| L1ME_ORF2 | TCGTGACCTTGGGYTAGGCAAWGATTTCTTAGATATGACACMAAAAGCAC | SEQ ID NO: 276 |
| MER89 | AAGCTCTGAATAAATAGCCTTTGCTTGTTCTCATTTGGKTGGTCTTCATT | SEQ ID NO: 277 |
| MER90 | CCTCGCTGCARCGAGCAATAAACCCAACTTGTTCAACCACAGGTGTGTTC | SEQ ID NO: 278 |
| CHARLIE3 | ACAGCAACCAAAACGAGATTACGGAGTAGACTGGACATAAGCAACACACT | SEQ ID NO: 279 |
| MER91_B | ATAATGACAATTTTCCAACAGATGGCAGTAAAGTGTCTTGAGGAAGGGGC | SEQ ID NO: 280 |
| HARLEQUIN | CCTGTACTTCTTCAAATGATAAAAAGCTTCATCGCTACCTTAGTTCACCA | SEQ ID NO: 281 |
| CHESHIRE | TGCCTTCCAAGCAATGAATATGCTCAATTNAAATCATATGCTCGTGATTG | SEQ ID NO: 282 |
| GOLEM_A | GAAATTGCCTAATGACGCATTTCTCAGAACGTATCCCCGTCGTTAAGCGA | SEQ ID NO: 283 |
| GOLEM_B | TCCTGCAAGCTCCATTCATGGTAAGTGCYCTATACAGGTGTACCATTTTT | SEQ ID NO: 264 |
| LTR34 | TGTGTCTGTGGCTCGCGTTTTTCCCGGACATGCCCTAAAGCTGGCTTAAT | SEQ ID NO: 285 |
| LTR35 | CGTGTTATTTCYATTACATGGRGAGCCCAGGAACCTGTGGTCNNTAAACA | SEQ ID NO: 286 |
| LTR36 | CCTGTACTTCTTCCCCCTAAGCTAGCTTTGGAATAAAAAGTCACTTTCTT | SEQ ID NO: 287 |
| MLT2A2 | CAGACTGAAGGCTGCACTGTYGGCTTCCCTACTTTTGAGGTTTTGGGACT | SEQ ID NO: 286 |
| HAL1 | GNAGGGATGGGGACTGCTTTTCGTNATAAGCCTTGTAGNACTATTTGACT | SEQ ID NO: 289 |
| MER66I | CTGGGCCCCTTAGATCAGGTATCCAGAGATTTTTACTCCTCCGGTGCTAG | SEQ ID NO: 290 |
| LTR37A | TTCCTTCCCCCACTGTGGAAAAAGCCAGTTTTGCNTCYATTTGCAAATTC | SEQ ID NO: 291 |
| LTR37B | GGGAATGTACCTNTGTTGACTTTGCTATTTACTATTTGATTAGGGCCCAG | SEQ ID NO: 292 |
| CHARLIE5 | ACGTTTTCTCACCGATATCACACTGCATATGAACAAGCTAAATTTGAAGC | SEQ ID NO: 293 |
| TIGGER5 | TTAAGGTAGGCTAGGCTAAGCTATGATGTTCGGTAGGTTAGGTGTATTAA | SEQ ID NO: 294 |
| TIGGER5_A | GGTTTCTACTGAATGTGTATCGCTTTCGCACCATCGTAAAGTTGAAAAAT | SEQ ID NO: 295 |
| TIGGER5_B | GTTTACCCTCGTGATCGCGCGGCTGACTGGGARCTGCGGYTCACTGYCGC | SEQ ID NO: 296 |
| LTR38 | ATCTCCCATCTGCTAGCATTTGATTAATAAAGCTGCTTTCCTTTCACCAC | SEQ ID NO: 297 |
| LOOPER | ATGACAGTTGATGAGCAGTTAGTTGCATTCAAAGGATATTGCCCATTTCG | SEQ ID NO: 298 |
| HERVK22I | GCGCCTGACAGACCTGTTGCTGCACACATCTGTACTCTTCAATCAACAAA | SEQ ID NO: 299 |
| MER51I | ACCACCCCTGGTCATTAAGGAGCTACCCTGTCTCCATTAGAHAGAGCAGG | SEQ ID NO: 300 |
| MLT1I | GAGCAGAGCCCCAGCCGACCCGCGATGGACATGTAGCATGAGCAAGAAAT | SEQ ID NO: 301 |
| LTR41 | AGGGGTAGTGGCTGCTCCTTATATCTGCTATTCCTATATTCTTTAGAGTT | SEQ ID NO: 302 |
| MER52A | CAATAAAGCTCCTCTTCGCCTTGCTCACCCTCCACTTGTCCGCGTACCTC | SEQ ID NO: 303 |
| MER52B | TCTCCTCTGAGCTGTTCTATCGCTCAATAAAGCTCCTCTTCATCTTGCTC | SEQ ID NO: 304 |
| MER52C | AGGATGGCCAGAGGACAAAGRGGGCAGAGAGACAATGGGACWGGATGACC | SEQ ID NO: 305 |
| MER94 | GCCTGGGACAGTCCTGGTTTATRCCTGTTGTCCTGGCGTAATTATTAATA | SEQ ID NO: 306 |
| CHARLIE6 | GAGGGGNAACCACACAAAAAGAGNAGGCTAATAAGTTGGCCAAAATAAGC | SEQ ID NO: 307 |
| LTR39 | TTTCTCCCGCTGCAAAATCTCGGTGTSGATGTTTGGTTTTACTGCGCCGG | SEQ ID NO: 308 |
| LTR40A | TCTCTGACCCAGGAGTCTCGTGTCTTCTGCCAGCATCCATGAAACTGTGG | SEQ ID NO: 309 |
| LTR40B | TCTCTGACCCAGGAGTCTCATGTCTTCTGCCAGCATCCATGAAACTGTGG | SEQ ID NO: 310 |
| HERVL_40 | TGCTTGGATGTCCTGTTGATAGTAGCCTTAATTAAATGCTNTATGAGACA | SEQ ID NO: 311 |
| LTR9B | GTGTCGTTTTATCTAAATCGGCGCGAGGACCAAGGACCCTGGTGTTCCTC | SEQ ID NO: 312 |
| HUERS-P3 | CTCCAAATGGTGCTGCAGACCGAACCACACATAGACACGCCATTCTTCCA | SEQ ID NO: 313 |
| HUERS-P3B | GAGATSAAATCAAAATCATTGACAGGCTCAGGGAAAATGCCGGCTTCAGC | SEQ ID NO: 314 |
| HUERS-P2 | TAGACACAGGNAAGAGACCTGGGAAGCTTNAGTAGCCACCGTGTAAGCCC | SEQ ID NO: 315 |
| LTR20B | TTCGCTCCAACCTCACCCTTTGTGTCCATGCTCCTTAATTTTCTTGGTCG | SEQ ID NO: 316 |
| HERVG25 | CTRAGRACCCTTAAACCAGCCTCRRGARAARTCCTAACTGCTGTTNCCTA | SEQ ID NO: 317 |
| LTR42 | CTTCTTTCTTTGGAATCCCAACTGGCCCCATCTCAGGANGGTTTGGGGYA | SEQ ID NO: 318 |
| LTR43 | TTCYTTTGCAATAAATTRCTCTATGCTGCATCTCCTTTGCTGTGTGTCTC | SEQ ID NO: 319 |
| LTR44 | GTGTGTCTTCCCAGGTCAATCCTCACATTTGGCTTCCAATAAACCTTTAT | SEQ ID NO: 320 |
| MER95 | GTCTCCCGGTTCGCGARCTGTWCTTTCTCTYATTGTATGCACAATAAACT | SEQ ID NO: 321 |
| L1MC5 | TAAATGACACCATRGGGATGCAATCAGCAAAATCCAGACTGTGGGAAACT | SEQ ID NO: 322 |
| MLT1J | ATGGAGCAGAGCTGCCATACCAGCCCTGGACTGCCTACCTCTAGACTTCT | SEQ ID NO: 323 |
| HERVFH21 | CAAGACATGATGCTACTCCAAGAATACCGACGGCTCCAGGAACAGCAGTC | SEQ ID NO: 324 |
| ZOMBI_C | AAACTCATTTGGCAGCAAAACCTGACCTGAACTGATATGAGGCTATTTAT | SEQ ID NO: 325 |
| MER96 | AATTTAAGGAGGCACTCACTCTCAGGGTCGTGCAAGTGCAGGGTCGGCAT | SEQ ID NO: 326 |
| LTR45 | GCCCACCTCCTGTCTCCTTGCTGGCCGGTTTTGCAATAAAGCCTTTCTTT | SEQ ID NO: 327 |
| LTR46 | TCTGGCATTAAGCTGGTCCCCCACYTYYRCAGGTTTTNTGCTGGATATAA | SEQ ID NO: 328 |
| MER99 | GCTTTCAACTTGATGTCAGTGGATTCCTTCGAATCAGTAATGTCTCTATG | SEQ ID NO: 329 |
| RICKSHA | AATACGGTTCGTCTGCTCATAACTGTTATACCCGTGCGACTGTCATTAGT | SEQ ID NO: 330 |
| MER96B | CTCAGGCTCCAGTATGAGTNGACACTGCACAGTTRCTGATCCTGTATTTA | SEQ ID NO: 331 |
| MLT1K | TCTTGCCACCACGNGGAGAGAGCCTGCCTGAGAATGAAGCCAACACAGAG | SEQ ID NO: 332 |
| HERVK3I | CCCTTGGACCAGTCTAAAGCACCACATTAACATCTTATATGTAGTCCTTG | SEQ ID NO: 333 |
| LTR22A | CGCTGCATACCTGTGTCTGAGTACTCATTTCATCCATCGGTCGGCCAGGG | SEQ ID NO: 334 |
| LTR47A | ACACAGACGTGGCTTCTGTTTGTAAGTCCCTATTAAATGTTTCTTTCTGA | SEQ ID NO: 335 |
| LTR47B | TCCTTCTGCGTTTGGGGGTCATTTTGCATATACGGCCCTTTCACGAAACA | SEQ ID NO: 338 |
| MER101 | TTCGTTTTACACCGAAGGCTGCATCTCCCCGGTTTGCAAACTGTTCACTG | SEQ ID NO: 337 |
| LTR48 | CAGTTCATTTCAGCAAACCTTCAGAGGGGACAGAGGGGAAGCTTTCCTTT | SEQ ID NO: 338 |
| LTR48B | TAATCATTCTCCTCTGTGATTCCCCCATGCTATGCACGTTAAAATAAATT | SEQ ID NO: 339 |
| LTR49 | TGCCTTTTGTCAGTTGATTTTTCAGCGAACCTTCAGAGGGCGAAGGGGAA | SEQ ID NO: 340 |
| LTR8A | CTCTTTCTTTATTGCAATGCCATGGTCTTTGTCTGTGCAGCGGGCAGGAA | SEQ ID NO: 341 |
| MER41E | GTAGAAGCCCCAAACCCYMTTGGCGCAACTCWCTCTCTTGAGTATGCCCG | SEQ ID NO: 342 |
| MLT2E | TCCCCCCTCCAGACCTTCACTTCCCCAGCTCCTCCCACAATTGTATAAGG | SEQ ID NO: 343 |
| LTR50 | TCTCTGTTAAAATAACTGGTGTGGTTTCTGTCTTCTCCTGACTGGACCCT | SEQ ID NO: 344 |
| LTR51 | TCTTTGAAGAGAGAGCGCCTTTGGTCTATGCCAGAGACTATCTCTTCCCA | SEQ ID NO: 345 |
| MER103 | GTGCATTGTGAATCTCCAAGAGGGGAAATATAGTATGCAGTRTTTCCCAA | SEQ ID NO: 346 |
| MER104 | TTAACATCTCTGAAATCGGGATGCATCTTACAATCGATGGCATGTCATAG | SEQ ID NO: 347 |
| CHESHIRE_A | ACAACGGCAGAGTTGAGTAGTTGCGACAGAGACCGTATGGCCCGCAAAGC | SEQ ID NO: 348 |
| CHESHIRE_B | ACAACGGCAGAGTTGAGTAGTTGCGACAGAGACCGTATGGCCCGCAAAGC | SEQ ID NO: 349 |
| HUERS-P1 | ATCTGCTCTTCGCCTTGCCCAGAGACCCCACTGTGAATTACCATTTGGAG | SEQ ID NO: 350 |
| LTR45B | GTATTGGCTTCGCATCAGGCAGCAGNNAGCCCATTGATTGCTTRGTAACA | SEQ ID NO: 351 |
| LTR52 | ATACCCTCTTGGTGTGTGTGTGGCATCATCAGTCTTAACATCCAAACCAA | SEQ ID NO: 352 |
| MER105 | GCCCTAAGGCATCCATTGTATGTAATGAATTAACTTCTCTCCTATGCATC | SEQ ID NO: 353 |
| LTR53 | CATCTGTCCAGTGTTGGGTGTCATGTGTTTARCCATCCCCATAACCCTAG | SEQ ID NO: 354 |
| LTR54 | TATAAAGCCAACCTCCTCTGCTCAGCTCATYGGAACACTCATTCTATTTT | SEQ ID NO: 355 |
| MER106 | TGTGGTATTAAAATTTCATGGNGGGGGGGGGTGATTAGGAAAAAAATGTC | SEQ ID NO: 356 |
| MER107 | TTCTACCTTATCACTAGAGACAGAAACTAAAACCATGGCTTCAGGCTGCT | SEQ ID NO: 357 |
| MER44B | ACTTAATAATGGCCCCAAAGCGCAAGAGTAGTGATGCTGGCATATTGTTA | SEQ ID NO: 358 |
| MER61I | CTACTGACAGCAGGGGAGATAGGGCATACGTGGGTAGAGCGGATAATTCC | SEQ ID NO: 359 |
| HERVL68 | CCCTGGAAGGCTTTCAGGTCAGCTTCAACTTACTGGCCAGAGTTGTGCTG | SEQ ID NO: 360 |
| MER83B | CCTCTTTGCAGACAGCCCCTTCTCTGCTGTGCTGCCCGTTGCAACCTTGC | SEQ ID NO: 361 |
| MER83C | GCACGTAGCCCCCTCCAGTACAACCCTATAAAACTTCCCTCCAGCCCCTG | SEQ ID NO: 362 |
| MLT1L | GAAAGAACCTGGGTCCTTGATGATATCGTTGAGCCGCTGAATTAACCAAC | SEQ ID NO: 363 |
| MLT2F | ATCAGACGCARAGACAACAGCCTTACAGAGACTGCTTAACCAGCTCCCAC | SEQ ID NO: 364 |
| LTR55 | TCATATCTTTTTCCTTGATCAGCCCCCAAATCCCTTRAACCCCCTTCACA | SEQ ID NO: 365 |
| LTR56 | CTCTTTTTTGCCTTTAAAAATCCACTTGTAACTGCTGCTAATTGGAGTGT | SEQ ID NO: 366 |
| LTR57 | GAGTGCCCTGTATGTAAGTCCTAATAAACTCATCTACTTATCAAGCTGGA | SEQ ID NO: 367 |
| LTR58 | AGCCGCAAGCCTATTAAACCTTGCCTGAGAAAATCGGTTTGGCCTGGTGT | SEQ ID NO: 368 |
| LTR59 | ATTTTTCCTRGRTGTGCCCTCAAGCTGGCTCAGTAAACCTCGATGNTTTG | SEQ ID NO: 369 |
| MER4BI | CTGANAGGATAAAGATACCTCGTGACAAAGCCTCCTGGGTATAATACTCC | SEQ ID NO: 370 |
| MER50I | AAAATGGCTTCCCTGGGTTCTTCCCTTTTTAGGCCCACTTGTTAGTCTCC | SEQ ID NO: 371 |
| LOR1I | TCCAATTACAGGTGTGACGTTTTCATTCCTCATCATTATCCCACAACGCC | SEQ ID NO: 372 |
| LTR26E | TCGGTGTATTGACTTGCCGCGCATCGGGCAACAAACCTATTACGGTCACA | SEQ ID NO: 373 |
| LTR16A1 | CTGCCCTATCCTGCTTCCCTCACTCCCTTACAAGTTTCTCCTGAGAGCAC | SEQ ID NO: 374 |
| LTR24B | TCTTTGGAATCTGTGYTTCCNGGGTGGNCCATCNTCAAACTTTGCACTTG | SEQ ID NO: 375 |
| LTR16D | CCCGCTCCTGCTCCCTCCCCTTTTATCTTTCACAGGNTTTCCCCTAATAA | SEQ ID NO: 376 |
| LTR60 | CTTCAARAAAAATCYGACATCATAAAAACCCCGTGCAGACTCTCAGGGCT | SEQ ID NO: 377 |
| MLT1E1 | GTAGGCAGAATTCTAAGATGGCCCCCAAGATTCCCACCCCCTGGTGTACA | SEQ ID NO: 378 |
| MLT1J1 | TAGCCAACGGAATGTAAGCAGAAGTGATGTGCGCCACTTCCAGGCCTGGC | SEQ ID NO: 379 |
| MLT1J2 | CCTGAGTCACTACNTGGAGGAGAGCCACCCACACCCGACCAGAACCCNCA | SEQ ID NO: 380 |
| LTR1B | TCRGCTRGGGRCRGTCAGAGARGAGNTCAGCCGCTGGAYNGCCAAACTCC | SEQ ID NO: 381 |
| MER109 | TGTCCRTCATTNCTGGCATNGTCAGGACTAGGTAMGGTCTCGDCCAACTG | SEQ ID NO: 382 |
| MLT1E2 | GCCCCCCAAAGATGTCCATGCCCTAATCCCTGGAACCTGTGAATATGTTA | SEQ ID NO: 383 |
| LTR22B | CACTGGCTGGTCGGCAACTGTTTACAGCACTCTCCTGGGAGTCTGTAAGC | SEQ ID NO: 384 |
| MLT1G1 | TTTCCAAAGATGGCCGCAACAATATCTCCCATCCCACATGCTCTTCTTAC | SEQ ID NO: 385 |
| L1MCC_5 | GCCCATTTCCAGGCATAAATACTATTTACCTCAGTCTCTACTGTTCTTCT | SEQ ID NO: 386 |
| MER110 | CTCGCCTCACTGTGCCCACCAATCCAAAGCTATTATGTCATAAACTCTGC | SEQ ID NO: 387 |
| HERVK11I | CAAAGAATCCTGCGTCAAAATCGAGAGAACGAACAAGCCTTCATCGCCAT | SEQ ID NO: 388 |
| HERVK14I | AATAAAAAGGCTGGACAAGATATATGGTGGAGGGATGCACATACAAAGAG | SEQ ID NO: 389 |
| HERVK13I | CAGGCGTCTCCACGGAGTCCAATGAAAAACTCGAAGCCAGCGACAAGCAA | SEQ ID NO: 390 |
| HERVK14CI | CTCATAGCTCCTATAATGCCATTGAACACCAGTGAGAGACGATTAGACGT | SEQ ID NO: 391 |
| LTR14C | ACCGCCACTGCTACACATCTTATCGAATGACTCACGAGTTCTCCTTCACT | SEQ ID NO: 392 |
| LTR61 | ATCCACTGAGCTGGTGCGTACCTTAAAATAAATAACAATCCTCCTGTATT | SEQ ID NO: 393 |
| HERV49I | CTCAATTTGTTTTCTCCCCTCCTTTGCCTATCTCTATCTAACAACCTCTA | SEQ ID NO: 394 |
| HERV15I | ATAGAGGCAGTAGTAACCCGAAACACTACCATGCTATTGACGGCATTAAC | SEQ ID NO: 395 |
| LTR62 | CAAANATGTGTGGACCTGGITATCTCTGAGCTTGCRCTGCTCACGACACA | SEQ ID NO: 396 |
| LTR64 | GGCTATAGGCNTYCCTCAGTCTACAGTCCTCAGTAAGACTTCTGAATAAA | SEQ ID NO: 397 |
| MER112 | CCAGACCAGTGGCTTTCAAACTTTTTTTGACTATGACCCACAGTAAGAAA | SEQ ID NO: 398 |
| MER113 | AAGCACCAAACTGAGACTTTCTCCTTGATGTAATCAGAAGGATTGAAAGA | SEQ ID NO: 399 |
| MER110A | TTACCCAATCCTAATCAAGCCCCTACATTGAAAGACCTGCCTTAAATCAG | SEQ ID NO: 400 |
| LTR33A | CTTCTTGCTGTTGCTAATCTCTGGGTTGCCTCACCATTGNTTCCCTGTTT | SEQ ID NO: 401 |
| MLT1F1 | CCCCGGCCGACATCTTGACTGCAACCTCATGAGAGACCCTGAGCCAGAAC | SEQ ID NO: 402 |
| SATR1 | ACACCCCCCCCSTACVCCCACMCCCCCTGTGATATTGTTCGTAATATCCA | SEQ ID NO: 403 |
| MER115 | TTTAAATATTTAGACATATGGTATGTGGGCCTCCATTTGTACTCTTGCCC | SEQ ID NO: 404 |
| MER117 | GCACAGGAGGGGGAAGTAGCAGCANATATGCTATGTATTTGCCATCCCTG | SEQ ID NO: 405 |
| MER20B | TAGGTGCAAGCATCTGACTACTTCATTATGTCTTCTAGTGTAGTCATGCC | SEQ ID NO: 406 |
| LTR65 | TCCATGGTTCCTCTGGTGTGCAGTCTCCCTCATTGCAATAAGTCAATAAA | SEQ ID NO: 407 |
| LTR38B | TGAAGYGGTTGCTTTGGATAGGAATCYGGCCRCTTCCCCATTACTAGTTT | SEQ ID NO: 408 |
| CR1_HS | GGATTGACAGCAGATCAMGGGAAGTGATTATACCCCTTTACAATGCCTTG | SEQ ID NO: 409 |
| L1ME4 | GTGGGATGGACAGGGATGGGAGGGACTGACTTTTCACTGTATACCTTTTT | SEQ ID NO: 410 |
| MLT1H1 | TGGACCCTCCAGACCAGCCCATCTGCCAGCTGAATACCACTGAGTGACCT | SEQ ID NO: 411 |
| LTR2B | GGGACAGAAATTGTGCACTCGGGGAGCTCGGATTTTAAGGCAGTAGCTTG | SEQ ID NO: 412 |
| MER101B | CCAGAAACCACCTCCCCACAAGCCCACTAGAAACAAACATCTGACAGAGA | SEQ ID NO: 413 |
| MER45R | TAGCCNATAAAATACTCTTAACAGCTCCAGNAACAGTTGCATCAGCAGAA | SEQ ID NO: 414 |
| MLT1G2 | TTTAAAACATGGCCGCAAATTCTTTGACACTCCTCTCATTGAGANGTGGG | SEQ ID NO: 415 |
| MSTA1 | CTTGCTTCCTCTCTCACCATGTGATCTCTGCACACGCTGGCTCCCCTTCC | SEQ ID NO: 416 |
| LTR6A | GAATTCGTCTCAAAGTGTGGCGTTTCTCTATAACTCGCTCGGTTACAACA | SEQ ID NO: 417 |
| L3 | GGTCTGGAAACCATGTCATATGAGGAACGGTTGAAGGAACTGGGGATGTT | SEQ ID NO: 418 |
| LTR66 | TGCCATTTACGTGGGATAAAGCTTGTTTACCCTTAAAGGTATTGTGTGTG | SEQ ID NO: 419 |
| PRIMA41 | ACCTTTTGTCGGAACTCGGAGTTATGAACGACCCTCACCATACCGATGCT | SEQ ID NO: 420 |
| MARNA | TATNGCCTCCCAAGGTGACTACTTTGAAGGGGACAACACTCATTTGGATG | SEQ ID NO: 421 |
| MER119 | TTACTGAGACACTAAGGGCGCCGTGAACCGAGAAAGTTTGGGAACCTCTG | SEQ ID NO: 422 |
| LTR67 | GTTCTCCAGCCCTCCCGGAGATTCTGTGAGCTACCCAATATCCTTTAATA | SEQ ID NO: 423 |
| L1M3DE_5 | CGGGCNGATTGGTGAGATCCNTCTCCTACACGAGGCCAGTCTGACAAGAC | SEQ ID NO: 424 |
| RICKSHA_0 | CTCTTATGGACTATCTGCGTGCAATTGCCCATAATCTATCCCTGTAATAT | SEQ ID NO: 425 |
| MER4E | AGGGGTCTGGGGAGTCATGCCCTACAAACCATAAATTCTCATCAGATGGG | SEQ ID NO: 426 |
| MER104A | ACCTTTCGCGTTTCAGTTAACAAACCATTTAAGGACCATTTGAGGAAGGA | SEQ ID NO: 427 |
| LTR40C | TGCTCATGCTGCTTGCTGTGYCATGAGTAATAAAGTCCTTTGTCTCTGAC | SEQ ID NO: 428 |
| LTR54B | TGCTCAAGCTACTTTACAAAAGCCAAACTGCTCTGCCATGCCCAGCGGAG | SEQ ID NO: 429 |
| MIR3 | GGAAGCAGTATGGTATAGTGGAAAGAACAACTGGACTAGGAGTCAGGAGA | SEQ ID NO: 430 |
| MLT1G3 | CCAGCTGTCAAGTCATCCCCAGCCTCTNNCAGYCMTCCCCAGCCTTCAAG | SEQ ID NO: 431 |
| MSTA2 | CCACTTCCCCTTTGACCTTCTCTGCCATGTTATGATGCAGCATGAAAGCC | SEQ ID NO: 432 |
| L1MD1_5 | TTTGAGAACTGAACTAAAGGATAGACCACTACCCAGGTCCCAGACTGGCC | SEQ ID NO: 433 |
| LTR10E | ARTGCTAATTTTTCTTTGCAGCACCGAGGAACAAGCATTCTGTTTCTAAA | SEQ ID NO: 434 |
| LTR24C | TCTCTGGAGTCTGTGTTTCCTGAATGGCCATTCCCAGCTTTTNACTTGAA | SEQ ID NO: 435 |
| MLT1C1 | TGGAGTGATGCAGCCATAAGCCAAGGAATGCCAGCAGCCAAGCCACCAGA | SEQ ID NO: 436 |
| MSTD | GTGGGTTTGTTATAAAAGNAAGTTCGGCCCCCTTTTGCTCTCTCNCTCTC | SEQ ID NO: 437 |
| LTR68 | ATCTTTACGTCATATACATTTCCATGTCTCAGGAGGCTAGGGCTTTTTAC | SEQ ID NO: 438 |
| L1MED_5 | TAAAAACCCAGTGGATAGGTNAAACAGCAGATTAGANACAGCTGAAGAGA | SEQ ID NO: 439 |
| L1ME5 | ACTGAAAGGAAATATACACCAAAATGTTAACAGTGGTTATCTCTGGGTGG | SEQ ID NO: 440 |
| TIGGER6A | TAGAAGAAATAGCTGACCGTGGGAATGTTGACACTGCCGCCATTTGAGAG | SEQ ID NO: 441 |
| MER51C | AGACCAAATCCTTCATCCAGATAAGGGGTAGCCAATAGAACCTCAAAAAG | SEQ ID NO: 442 |
| LTR6B | CCGGCTAAATAAACGGACTCTTAATTCGTCTCAAAGTGTGGCGTTTTCTC | SEQ ID NO: 443 |
| MER21A | TCCACAGTTCCTGGCTCATAACTCCCATAGCCCTTGTTACAGTCTTTTGT | SEQ ID NO: 444 |
| MER34B | CCACAAGTTGCTGCCCCTAGAGACTCAAAGTCCTTTTCCTTTGTCTTGTC | SEQ ID NO: 445 |
| LTR3B | AGTTTCTTTTGTCTTAAGTTTTCATTTCTGCGTTCGTCCCCCTTCGTTCA | SEQ ID NO: 446 |
| MER54A | AGGCGGTTGTATAAGGCAGATATCTGGATCGACCACATTGAGGAACTGGG | SEQ ID NO: 447 |
| MER74C | GCCTTTCATCTATCCGAGTGTCANTGTGTTGTGTCCCGCCATCAAAAGAA | SEQ ID NO: 448 |
| ERVL | AAGAGTAAACATCACTCAAGGACTTTACCTCCTCTTCTCGGGAAGGGGTT | SEQ ID NO: 449 |
| HERVL74 | AAATACCCCNAATAATTGATGTCAAAACTGACGTCAAGACANAAAGGGGT | SEQ ID NO: 450 |
| MER83AI | TAAGTCCCAACTCAGGGATTTAGGTCCACGTAACCTCCTGACCGACTAAC | SEQ ID NO: 451 |
| MER83BI | TCTCCGATGAGTTCTTTCCTCCAGCAAGATCCAATATCCTAAGTCCCACA | SEQ ID NO: 452 |
| MER84I | ATTTTCCCTTTCTTGAGACCCCAATAGGCAGCAGGTAGACATGAGCATGG | SEQ ID NO: 453 |
| LTR75 | TAATAAACTGTCTGAATCTAAAAGTGGCTCGTTGTATCTTTACCAGCCGA | SEQ ID NO: 454 |
| L1PA7_5 | CACCGAGCTAGCTGCAGGAGTTTTTTTTTTTCGTACCCCAGTGGCGCCTG | SEQ ID NO: 455 |
| L1PA13_5 | CTTTAGCCCTAGGGGAACTGTCGGACCTGAACTCTGCAGGGCGGTCTTGC | SEQ ID NO: 456 |
| L1M1_5 | AAGAAACAAATAACATACAATGGAGCTCCAATACGTCTGGCAGCAGACTT | SEQ ID NO: 457 |
| L1M2A_5 | CATGTCAGACCCGACACCAAGAGGGATCCCCTCGGCTAAGTCTCCCCATT | SEQ ID NO: 458 |
| L1M1B_5 | CCCATTCGGGACGGGCAGCGCTCTGATTGTTTACTAGAGCCGAGGCAAAC | SEQ ID NO: 459 |
| L1MB3_5 | AAAGGGGTGGGGATGGAGCTGTAAAGGAGCAGAGTTTTTGTATGTTATTG | SEQ ID NO: 460 |
| L1MDB_5 | CACAAAAGTAGGCCAGGACCTGCATGCTAAACCTAAACAGGGTGACTGCC | SEQ ID NO: 461 |
| L1HS | CACAGGAAGGGGAATATCACACTCTGGGGACTGTGGTGGGGTCGGGGGAG | SEQ ID NO: 462 |
| L1PA3 | AACACATGGACACAGGAAGGGGAACATCACACTCTGGGGACTGTTGTGGG | SEQ ID NO: 463 |
| L1PA4 | AACACATGGACACAGGAAGGGGAACATCACACACCGGGGCCTGTTGTGGG | SEQ ID NO: 464 |
| L1PA5 | GAACACTTGGACACAGGAAGGGGAACATCACACACCGGGGCCTGTTGTGG | SEQ ID NO: 465 |
| L1PA6 | GAGAAATACCTAATGTAAATGACGAGTTGATGGGTGCAGCAAACCAACAT | SEQ ID NO: 466 |
| L1PA8 | AGGACAAATACCTAATGCATGCGGGGCTTAAAACCTAGATGACGGGTTGA | SEQ ID NO: 467 |
| L1PA10 | ATAGCTAATGCATGCTGGGCTTAATACCTAGGTGATGGGTTGATAGGTGC | SEQ ID NO: 468 |
| L1PA12 | CTTAATACCTGGGTGATGAAATAATCTGTACAACAAACCCCCATGACACA | SEQ ID NO: 469 |
| L1PA13 | TACCTGGGTGATGAAATAATCTGTACAACAAACCCCCATGACACAAGTTT | SEQ ID NO: 470 |
| L1PA14 | GGGAGAGGAGCAGAAAAGATAACTATTGGGTACTGGGCTTAATACCTGGG | SEQ ID NO: 471 |
| L1PA16 | TGGGTGATGGGATCATTCGTACCCCAAACCTCAGCATCACGCAATATACC | SEQ ID NO: 472 |
| L1PB2 | ATCTCAGAAATCACCACTAAAGAACTTATCCATGTAACCAAAAACCACCT | SEQ ID NO: 473 |
| L1PB4 | KTACACTAAAAGCCCAGACTTCACCACTACGCAATATATCCATGTAACAA | SEQ ID NO: 474 |
| L1MA1 | ATTCTCCATGATGTGCTTATTTCACATTGCATGCCTGTATCAAAACATCT | SEQ ID NO: 475 |
| L1MA3 | GCTGGGAAGGGTAGTGGGGTGGGGGGGAAGTGGGGATGGTTAATGGGTAC | SEQ ID NO: 476 |
| L1MA4 | GGAGGGGGGGAATGAAGAGAGGTTGGTTAATGGGTACAAAAATACAGTTA | SEQ ID NO: 477 |
| L1MA4A | GAGGACTTGAAATGTTCCCAACACATAGAAATGATAAATACTCGAGGTGA | SEQ ID NO: 478 |
| L1MA5A | TGGGAAGGGTAGGGGGAAGGGGGGGATAGGGAGAGATTTGTTAAAGGATA | SEQ ID NO: 479 |
| L1MA6 | ATAGGAGGAATAAGTTCTGGTGTTCTATTGCACAGTAGGGTGACTATAGT | SEQ ID NO: 480 |
| L1MA7 | ATGGGGAGATGTTGGTCAAAGGGTACAAAGTTTCAGTTAGACAGGAGGAA | SEQ ID NO: 481 |
| L1MA8 | TGCTNATGGTCCCATGACTGGCCACTCTGTGAACACAGTAAACAAGTTTG | SEQ ID NO: 482 |
| L1MB1 | GAAATGGGGAGTTGCTGTTCAATGGGTATAAAGTTTCAGTTATGCAAGAT | SEQ ID NO: 482 |
| L1MB2 | GGGTATAGAGTTTCAGTTTTGCAAGATGAAAAAGTTCTGGAGATCGGTTG | SEQ ID NO: 484 |
| L1MB4 | TGGTGATGGTTGCACAACAMTGTGAATGTACTTAATGCCACTGAATTGTA | SEQ ID NO: 485 |
| L1MB5 | AGGGGGAATGGGGAGTGACTGCTTAATGGGTACGGGGTTTCCTTTTGGGG | SEQ ID NO: 486 |
| L1MB8 | GGAATGGGGAGTGACTGCTAATGGGTACGGGGTTTCTTTTGGGGGTGATG | SEQ ID NO: 487 |
| L1ME1 | GGTGGGGGNAGGGGATTGACTACAAAGGGGCATGAGGGAACTTTTTGGGG | SEQ ID NO: 488 |
| L1ME3 | ATAGTGGTTACCTTTGGGGAGGGTTATTGACTGGGAAGGGGCATGAGGGA | SEQ ID NO: 489 |
| L1ME4A | GACTGGAAGGAAATACACCAAAATGTTAACAGTGGTTATCTCTGGGTGGT | SEQ ID NO: 490 |
| L1MC1 | TTGATAGTGGGGGAGGCTGTGCATGTGTGGGGGCAGGGGGTATATGGGAA | SEQ ID NO: 491 |
| L1MD3 | ACCCATAACCCCAGTCTAATCATGAGAAAACATCAGACAAACCCAAATTG | SEQ ID NO: 492 |
| HAL1B | AGAGGAGAGGTGGAAGGAAGTATGAGAGTGCTAATNTCCTCATCTTTCAT | SEQ ID NO: 493 |
| L1MA9_5 | AGACCCAGGGTTCAGGCCTGTCCCAGTAGACCCCAGCACTAGGCTAGTCC | SEQ ID NO: 494 |
| L1MDA_5 | AAGAAGGAATCTTGGAACATCAGGAAGGAAGAAAGAACATAGTAAGAAGC | SEQ ID NO: 495 |
| L1MEB_5 | GGCAGAAACTGGAGGGGAGTCGACACCTGGAAGAAGGGAATWGCACGGAG | SEQ ID NO: 496 |
| TIGGER5A | TTAAGGTAGGCTAGGCTAAGCTATGATGTTCGGTAGGTTAGGTGTATTAA | SEQ ID NO: 497 |
| TIGGER6B | AGGCAACCCCATCAAGAACTTANGCGAAAAAAGATGTAGGATCACAAAGT | SEQ ID NO: 498 |
| TIGGER7 | TCGGATGGAACGCAGCATTAAAGTCACCCATATGATCAATGAAGGATTAC | SEQ ID NO: 499 |
| MER44D | CCTCACTTCATCTCATCACGTAGGCATTTATCATCTCACATCTATCACAA | SEQ ID NO: 500 |
| MER69C | ATCGACGAAGATAACATAAAACTCATAATACGCCACTACAACGAGGACAT | SEQ ID NO: 501 |
| MER106B | TATTTATGTTTGATCCTCAGTGCTTTGTGTGACTTGGGCTTTGAGAATTA | SEQ ID NO: 502 |
| CHARLIE2A | GATTGGTTTGACAATGAGGACTGGCTTTGCCAATTAGGTTATATGGCAGA | SEQ ID NO: 503 |
| CHARLIE2B | TTAATNCACCTTTTGTAAGCCCTATACTTACTAGTGGCCCAATACCTTCT | SEQ ID NO: 504 |
| CHARLIE7 | ACTTAGAACCAGACCTTCGAATCGCTGTATCACAAAGTGTTAAACCAAGA | SEQ ID NO: 505 |
| CHARLIE8 | ATTTATGTTACCTGCCTGGCCCCTGTAGGCATTTGAGTTTGCGACCCCTG | SEQ ID NO: 506 |
| CHARLIE8A | ATTTATGTTACCTGCCTGGCCCCTGTAGGCATTTGAGTTTGCGACCCCTG | SEQ ID NO: 507 |
| MER63D | ACAATGTAACGGCTACAGACACGACACACTTTTAAGTTTAATCTGCATTA | SEQ ID NO: 508 |
| MER97A | TGTTAAAAAATGATCCGCTCTGGGTGTCGAATACGCTAGGTACGCCACTG | SEQ ID NO: 509 |
| MER97B | CCAGTGGTATGNTTTWGTAGTTGCCTAAATTGTACCTTTTGCAGACGTTT | SEQ ID NO: 510 |
| MER97C | TGTTAAAAAATGATCCGCTCTGGGTGTCGAATACGCTAGGTACGCCACTG | SEQ ID NO: 511 |
| MER6B | GTTCTTGGAAACTGCGACTTTAAGCGAAACGACGTACAGCAGGTCCTCGA | SEQ ID NO: 512 |
| ZAPHOD | ATTGCCGGCCCATCAACAGAACACCCAGACATGTGCAATAATAATTAAAT | SEQ ID NO: 513 |
| TIGGER9 | GCCAGTCAGATTTCACGGCANTGCCAATGTTTCTGTCTGTACAGCGNTGT | SEQ ID NO: 514 |
| HERVL66I | CTCCTGTGCTTACCCTGTATCTGTAATCTATATCAACTATGCCTTCCCCA | SEQ ID NO: 515 |
| THE1A | TTTATCAGGGGTTTCCGCTTTTGCTTCTTCCTCATTTTCCTCTTGCCGCC | SEQ ID NO: 516 |
| THE1C | GTGTCCCCACCCAAATCTCATCTTGAATTGTAGTTCCCATAATCCCCACG | SEQ ID NO: 517 |
| MSTB | TGTTAGTTCACGCGAGATCTGGTTGTTTAAAAGAGTNTGGCACCTCCCCC | SEQ ID NO: 518 |
| MSTB1 | CTTCCTCTCTCGCCATGTGATCTCTGCACACGCCGGCTCCCCTTCACCTT | SEQ ID NO: 519 |
| MLT1AR | TCAGTCTGCTCCCTATCTTCGGCTGCCCGTTTAGNTGTGGCTCAAGTGGG | SEQ ID NO: 520 |
| MLT1CR | AAGGTGCGGCCTGGTTTCTCCTTGCTGCTTATAGTAAAATGCGAGAGGAA | SEQ ID NO: 521 |
| MER104B | CCTTTCGCGTTTCAGTTAACAAACCATTTAAGGACCATTTGAGGAAGGAA | SEQ ID NO: 522 |
| MER104C | TGAAGGCAGGAGAAATTGCCNAATCCCNCGGAATAGATGAAAGAAATTTC | SEQ ID NO: 523 |
| HSTC2 | TNATGTAGACTCCTTCGCAAGACTCCATCAGCGAACCATTTGACACTTTT | SEQ ID NO: 524 |
| L2A | ACGCTCTTCCCCCAGATATCCACGTGGCTSGCTCCYTCACCTCMTTCAGG | SEQ ID NO: 525 |
| L2B | CCTGCCACTCTGGGTTATMATTGTCTGTKNGCANGTCTGTCTCCCCCACT | SEQ ID NO: 526 |
| MER51D | TTTGTTTGGGACACCAAGAGCCTGGAACTGCACRGCACCAKCTGGTAACA | SEQ ID NO: 527 |
| MER5C | TGGACCAGTGCTAGTCTGCAAACTGTTTGTTACCAGTCCATGATAAGATA | SEQ ID NO: 528 |
| HERVK11DI | CCCGGTGCTGAAGTTTTAGACGGTATCTCTGAGGGGTTATCTAATCTCAA | SEQ ID NO: 529 |
| LTR69 | GAAAAGTCGCCCCTGGGGAAGCTGGTTAACTAGGACCACCCAAGACCCCC | SEQ ID NO: 530 |
| HERV30I | AAAAAAGGAGCTTGAACACTCAGAACCCTGAAATATGTTTAACCAATGGA | SEQ ID NO: 531 |
| HERV19I | CATAGCAGGAATAATGGTTACTAACAGAAAATAACACATGGGCCTTTCCA | SEQ ID NO: 532 |
| LTR19C | TCACTCTGTGTGTGTGTGTCCGCGACCTCGATCTCCTTGGCCGTGAGACC | SEQ ID NO: 533 |
| HERV46I | ACCCACTGCTTCAAAACCCAAACCCTGATTACAGCNCCCCTATTCGGCAG | SEQ ID NO: 534 |
| HERV52I | TNAATAAGACATGGCACATTTCAGTCATCCATCAAACATCAGGGGTGAAT | SEQ ID NO: 535 |
| MER89I | GCTTCTGCGCAGCCGCTCTCTCATCAGATGATCGCCATGATGATACAACA | SEQ ID NO: 536 |
| MER110I | GACAATGGTCTNTCCTTCAGNTCGGGNTGAAGAATGACCAAAGGAGAAAT | SEQ ID NO: 537 |
| MER21I | ATCCTTGTTTCGNGTAAGGGAATTCAGTGGTTGGAAANCAGGGAGTGGCC | SEQ ID NO: 538 |
| PABL_AI | GCGCTCAAAGGGTGAGTTAACTGGATCGTATGCCGGGAGCCTATTGTTTT | SEQ ID NO: 539 |
| PABL_BI | CTCGCGGTCCTGGCCATCCTTGNAGGCATGGGCATAACGTTATGTTGTGG | SEQ ID NO: 540 |
| MERS2AI | ACNCCCANGGGATTATCTACTCCCCTAAACAGCTATCTCTCTTCTAAAGT | SEQ ID NO: 541 |
| HERV57I | AGCCATGGCTATACGTTATAGACCTGTATAGTTCTTCCCCTCATACCCTA | SEQ ID NO: 542 |
| MER70I | GGGCATATGAAATGGACTAGCTTTGCTAAGGGGGATATCTGGGTTGGGGG | SEQ ID NO: 543 |
| HERV38I | CGGGATCGGTTTGGAGTGCTCCGTCTGCATCGGATCCGTCTGTGTTTGTG | SEQ ID NO: 544 |
| L1M2B_5 | CTTTCCCTACCCACTGCCACTACNYCTGACTCTGGGGCCAAAGCACATGC | SEQ ID NO: 545 |
| L1M2C_5 | ACACCCCAATGAACTGACACCAAGACCCATTTATACAAATAAGTTTTTCC | SEQ ID NO: 546 |
| HERVFH191 | CTGGAGCAGTCCTCCAAAATAGACGGGGATTAGATCTTATAACGGCTGAA | SEQ ID NO: 547 |
| HERV70_I | CTCAGTGGCAGATGGTAGAGGTCAAGAGAGGANGGACACTAGCAACCAGG | SEQ ID NO: 548 |
| LTR70 | TCTTTGCTCCCAGGTTAYAATCCTNAAGCTTGRCCCAAATAAACTGTCTA | SEQ ID NO: 549 |
| MER120 | AGATGTGGATACTCAAGATTTCTATTGGGGAAAACTGTGGTCCTTAGTAA | SEQ ID NO: 550 |
| REP522 | TGTATTGCTGGCAGCAGTGAGGTGGGTTAAGGGTGCTATCCGGGGCTGCA | SEQ ID NO: 551 |
| LTR71A | TTAAAAGTCTCGCTTCCACTGTTCTTCGTGTCTCTGAGTCCATTCTTTGG | SEQ ID NO: 552 |
| LTR71B | CATTAAAAGTCTCACTTTCGCTGTTCTCCGGGTCTCTGAGTCCATTCTTT | SEQ ID NO: 553 |
| LTR12B | CCCACCAGAAGGAAGAAACTCCGGACACATCTGAACATCTGAAGGAACAA | SEQ ID NO: 554 |
| MER121 | AGACACTTTTTTCCCCCTTAATTTTTAAACCCATGTGTATTTCAAGGGAA | SEQ ID NO: 555 |
| MER122 | TGCAGTTGGTGGCGACAGAGACTGTAGTGTGGCTGGAGTGGTAGGAAGGG | SEQ ID NO: 556 |
| LTR7A | AAAGCTTTATTGCTCACACAAAGCCTGTTTGGTGGTCTCTTCACACGGAC | SEQ ID NO: 557 |
| LTR7B | ACAGCCTTGTTGCTCACACAAAGCCTGTTTGGTGGTCTCTTCACACGGAC | SEQ ID NO: 558 |
| MER51E | GATTAGGCAGCAYACAGGCCACATCCTCACTCCTGTGATAACAAGACAGA | SEQ ID NO: 559 |
| MER41F | CAGGAGAATAGAAAATTCCAGGCAGCAGTTTCACATGACTAGCAAAAGGA | SEQ ID NO: 560 |
| LTR2C | AAGATAAATAGCCAGACAACCTTGGCACCACCACCYGGCCCTAGGAGTTA | SEQ ID NO: 561 |
| LTR38C | ACACCTCACTCTTGTTATTTTGGCTTCTTTCTACAAGCGGCAAGCAGCYG | SEQ ID NO: 562 |
| LTR72 | AACCTGTATTCTCATGGAGAGTCGTTTGTTACTCACCAGGYGAATRAACC | SEQ ID NO: 563 |
| MER65D | TAAAAGCTTCCCTTTACCCTCCCCTCTTCAGATGCATCTGTGGCTTGCCA | SEQ ID NO: 564 |
| ALR1 | TGAGGCCTTCGTTGGAAACGGGATTTCTTCATATAATGCTAGACAGAAGA | SEQ ID NO: 565 |
| LTR1C | GGTTCCAGCATTCATTCGCTCCGGTTCCCGCACTCACTCGCTTGCATGCT | SEQ ID NO: 566 |
| LTR45C | TCTCACAAGCAGAGGGAGTTTCAGCATTTCAGCAAGTTGTTTCTTTTCTT | SEQ ID NO: 567 |
| LTR76 | GATGTTAAGTCTGCTGGGTCTGAGTGCACTCAATAAAAGATCCTCCTGTT | SEQ ID NO: 568 |
| MER72B | TTTCACAATGCATCCCTTCCTAAAAACTGACCACCATCTCTGGACTGGTT | SEQ ID NO: 569 |
| ALR2 | GTGAAGGGATATTTGGGAGCTCATTGAGGCCTATGGTGAAAAAGAAAATA | SEQ ID NO: 570 |
| LTR1D | GTTCCAGCACTCATGCACTCCAGTTCCCACCTCGTTCACTCACATGCTCC | SEQ ID NO: 571 |
| MER34C | TCCTGGTCACCTCCCCATAACTGGCCTTCCCCACACCCTTCTTTCTTTGT | SEQ ID NO: 572 |
| MER50B | ACTCCCTAAACACACTGCGCGTGCTCAATTCCCAAGGGTAAGGAGGGCAC | SEQ ID NO: 573 |
| HERVP71A_1 | AATTGTGGCAGGAGTCTTAACAGCAGTGGGATGTTGTATTATCCCTTGTG | SEQ ID NO: 574 |
| LTR27B | TTTGCCCACCCTTTCCCGATTGATTCTTTCTGAATAATGCCTTTTAACCA | SEQ ID NO: 575 |
| LTR12C | CACCAGAAGGAAGAAACTCCGAACACATCCGAACATCAGAAGGAACAAAC | SEQ ID NO: 576 |
| LTR43B | CAGTCGGTGCTGTCTCACYYTTGAGCAGCCNYGCTCTGACTCAGCTGTCA | SEQ ID NO: 577 |
| LTR72B | CCCTTGTTAAATCCTCCTTGGTTGTGGTCATTGGACTGTCACCTGCCAAG | SEQ ID NO: 578 |
| LTR77 | GGGACAAGAACTCAGACCTTGCTAAACTAAGGAGTAAGAAGACTGCAACA | SEQ ID NO: 579 |
| L1PREC1 | GTCAAAGTGCTTCATTAAATGGGTCCTGTTCCCTGTGCCACCCAACTGGG | SEQ ID NO: 580 |
| MER2B | TCATTCACGTGGATTCAATGTAGTACTYGGTGTATGGCAAATTCAAGTTT | SEQ ID NO: 581 |
| MER93B | CTATAAAAGCCTCCCCCTTGCATTCCCTCGGTGGAGCTCCCGAACCACTT | SEQ ID NO: 582 |
| SATR2 | TGTACACCCTGTGATATTATTCGTAATATCCTAGGGGGATGTTACTCCTA | SEQ ID NO: 583 |
| GOLEM_C | GGGNAAATGANTGATATTCAGTAATGGTGCTGGGACATTTGGTTTTCCAT | SEQ ID NO: 584 |
| MLT1A | CCCCTCTAGAGGATGCAGCATWCAAGGYGCCATCTTGGAAGCAGAGASCA | SEQ ID NO: 585 |
| L1PREC2 | TGGCTGAACACTCCCAGTAACAGTGGCTCTGCGTTTCTCGGAGGTGGAGC | SEQ ID NO: 586 |
| BLACKJACK | CATCCAAACAAGCTGCGATATTCTACCCAACGATATAGAAGCTGTAGTTG | SEQ ID NO: 587 |
| L1M2A1_5 | GCCCACCCAACCCATCACAGCTTCCAGCAACACCAACATGGACTGCTTGG | SEQ ID NO: 588 |
| MLT1E1A | TGGAAGAGGATTCTAAGCCTCAGATGAGAACACAGCCCTAGCCAACACCT | SEQ ID NO: 589 |
| MER4E1 | TTCTTCCAGACCCTCCCAATCCTAAAGAGATTAACTAAGATCTGAATAGG | SEQ ID NO: 590 |
| PRIMA4_I | CGTGACCTCCTAGGATGAGCCTTCCTAGTGATGTGGGACCTAAAACTTCT | SEQ ID NO: 591 |
| PRIMA4_LTR | TTTAAATTTGGAGCCCTCAAAATCATCTTCGGAGAAAGGCATAGACCTGT | SEQ ID NO: 592 |
| L1M4B | AAAACAANCACNANGAGCCGGGGGNGGGGAATCAGTATCCAGAGTTGCTA | SEQ ID NO: 593 |
| L1PA14_5 | CACACAGACAGCAGATTAGGGCTAACCTGGCAAGGATACAGCTTGTCTGC | SEQ ID NO: 594 |
| LTR13A | TCTCTTTGTCTTGTGTCTTTATTTATTACAATCTCTCGTCTCCGCACACG | SEQ ID NO: 595 |
| HAL1C | AACCACAACATNAGAGGACCCANCACTCCTCCTACCACCAAAACAAAACC | SEQ ID NO: 596 |
| HERVIP10F | AGAGGCTCATAGAAATGGCACTTACTAAAACCTCCCTTAACTATCCTCCA | SEQ ID NO: 597 |
| MLT1F2 | CNGATCCTCCCCTCNAGTTGAGCCTTGAGATGAGACTGCAGTCCTGGCTG | SEQ ID NO: 598 |
| MLT1FR | TTTGGACCCCCAAAATTCTACTGGCAGGAAGCAGGCTGAGAAAACTACTC | SEQ ID NO: 599 |
| HERVIP10FH | CAGAGGCTCATAAAAACGGCACTTACTAAAACCTCCCTTAACTATCCTCC | SEQ ID NO: 600 |
| LTR10F | TTCCCTCCCTTGTCCAGGTGTGCGCTCACCATTGCTCCATCTGTGAGGGT | SEQ ID NO: 601 |
| MER34B_I | CTAAAGACACTTTGTGCTCAGACCTAGAAATCTTCTCAATTGGCTGCCAT | SEQ ID NO: 602 |
| MER57A_I | CTGGAAGGCCTATGCACCTAATAATAGAACCTCATGTATCTTCCGCTACT | SEQ ID NO: 603 |
| PRIMAX_I | AATTAACCAAGGCTTTTAAAATTCCTTGGCCAAAAGCTCTTCCATTGGTT | SEQ ID NO: 604 |
| MER75B | CATTTCCCGTTTGCCCCAAGAATACTCTTGTCTCTAATCCTAATGTAACA | SEQ ID NO: 605 |
| MLT2B3 | CCCAGGTGGTTTGGCATTTGATTAGAATGATTGGGCTGCCCCAGGTGTGT | SEQ ID NO: 606 |
| MER66C | AGGATCTGGTCCAGACAGGATAAAGTGAAGAAACNRGCAGGAACCAGCAG | SEQ ID NO: 607 |
| MER52D | CACNGCTCCACACCTGRCTTNNCCTTGGCAGGNNTGGATCNAGGNCCTTG | SEQ ID NO: 608 |
| MER41G | TGCTTTGCAATAAAAGCTTCTTGCCTTTCGCTTCATTCTGACTCATCCCT | SEQ ID NO: 609 |
| MER21C | AGGAGCATCTTTTGTTCTAATATTTGGTCTTTGACCCTAGTTCCTGACAC | SEQ ID NO: 610 |
| LTR20C | CCAACCTCACCCTTTGTGTCCATGCTCCTTAATTTTCTTGGTTGTGAGAC | SEQ ID NO: 611 |
| L1PBA1_5 | TCTGTTTGCGGGAGAAGTTTCTGACTTTACCTGGAGCTGAGTCAAKTTAG | SEQ ID NO: 612 |
| L1MB4_5 | AATCTCATGTCAAAAAAACACTAGCTGAACACAAGCTAAGGAACAGAGAC | SEQ ID NO: 613 |
| LTR73 | TTGACACTCACTTTCGGTTTTGTGTATTGGCTTCGTGACACCAAACAGGG | SEQ ID NO: 614 |
| HARLEQUINLTR | GGGAGGAGACCACCCCTCATATTGTCTTATGCCCAATTTCTGCCTCCAAA | SEQ ID NO: 615 |
| LTR12D | CACCAGAAGGAAGAAACTCCGGACACATCTGAACATCTGAAGGAACAAAC | SEQ ID NO: 616 |
| LTR12E | CACTCCTGAAGTCAGCGAGACCACGAACCCACCGGGAGGAACAAACAACT | SEQ ID NO: 617 |
| MLT2B4 | GTAAGAGAGAATTCCTCCTGCCTGACTGCCTTTGAACTGGGACATCGGTC | SEQ ID NO: 618 |
| MER9B | TAACAACATGTTTTTGCTCGCAGATAACAGCCAGAGCCTGTTTCTCTRCT | SEQ ID NO: 619 |
| SVA2 | GAAGTGACAGCCTTGTGTGTGATCTTTCTGCCCTCCCCAAGTTTGCATTT | SEQ ID NO: 620 |
| HERV39 | TCTTGCTGCTAAAACTGCATACAACAGCCACCCAGCCAAGAGGAATTAAT | SEQ ID NO: 621 |
| MLT1H2 | CCCAGCTGCCATGCTAAAAGAAGCTCAGGCTAGACTATTGGATGATGAGA | SEQ ID NO: 622 |
| LTR10G | GCTGAGAAAACTTTTGCCTGAGTGCTGGTTTCACTTTGCGGCACCAAGCA | SEQ ID NO: 623 |
| MER4A1 | CAGAAACTCAAAAGAATGCAAGGATTTGTCTCTCACCTACCTGTGACCTG | SEQ ID NO: 624 |
| MER4D1 | CTCTAGTATAGCATCACATGACAGATAGCAGGCCCTGAAAGAAATCAAAG | SEQ ID NO: 625 |
| THE1D | CNTCTCTCTCCTGCCGCCTTGTGAAGAAGGTGCTTGCTTCCCCTTTGCCT | SEQ ID NO: 626 |
| LTR5B | CCTCCGTATGCTGAGCGCCGGTCCCCTGGGCCCACTGTTCTTTCTCTATA | SEQ ID NO: 627 |
| MER46 | TTGAGTATCCCTTATCCAAAATGCTTGGGACCAGAAGTGTTTCGGATTTC | SEQ ID NO: 628 |
| CHARLIE4 | GTGACTCCACATGTTAATGGTCTTATTCAAGCTAAGCAGCATCTACTATC | SEQ ID NO: 629 |
| CHARLIE9 | CGTTGCAACGTGCACAGTTCATGCTAAGGATCCGTGCGATGCACTCTGAT | SEQ ID NO: 630 |
| TIGGER8 | NGTCNATTGTTTGACTTTCACACATTCGACTTCCATACACGTTTTCAGGA | SEQ ID NO: 631 |
| MER5A1 | TACTGAATCAGAATCTGCGTTTTAACAAGATCCCCAGGTGATTCATATGC | SEQ ID NO: 632 |
| KANGA2_A | TTGGCCANAAACTTTTNTTGAATCTTCTCATTGGGAAAAATTGGGAGATC | SEQ ID NO: 633 |
| FORDPREFECT | TTCACGTGCACTGATTGGACAATAAACAAATACGTAAGTACCTCTTCTCT | SEQ ID NO: 634 |
| FORDPREFECT_A | ACTTAGAAAATTTCGAGGAAGGCACTCCAAAGCACGGGGTCCCCTGAGGC | SEQ ID NO: 635 |
| LTR16E | ACGCATCACCTTGCATTGCTTCCCATCCTTCCCTGCCTCACTTCCCTTTT | SEQ ID NO: 636 |
| L1PA17_5 | CGAAGCCAAACGATCATACACAACATACACCACAGTCATACCCTCAAGGG | SEQ ID NO: 637 |
| CHARLIE10 | AGTAGCGCTGTCATCAATCCAACCTAGATTAGATAAGTTAACAAGCAAGA | SEQ ID NO: 638 |
| THE1B | CGCCATGATTGTGAGGCCTCCCCAGCCATGTGGAACTGTGAGTCCATTAA | SEQ ID NO: 639 |
| MSTA | ATGATTGTAAGTTTCCTGAGGCCTCCCCAGAAGCCGAGCAGATGCCAGCA | SEQ ID NO: 640 |
| MSTC | ATGCGGCCCCTCGACCTTGGACTTCCCAGCCTCCAGAACTGTAAGAAATA | SEQ ID NO: 641 |
| MLT1A | GCCGTCTACGAACCAGGGAATGAGCCCTCACCAGAAACTGAATCTGCCGG | SEQ ID NO: 642 |
| MLT1B | GCCATCTACAAGCCAAGGAGAGAGGCCTCAGAAGAAACCAACCCTGCCGA | SEQ ID NO: 643 |
| MLT1C | CATGGAACAGATTCTCCCTCACAGCCCTCAGAAGGAACCAACCCTGCCGA | SEQ ID NO: 644 |
| MLT1D | TAGCCCAGTGAGACCCATTTCGGACTTCTGACCTCCAGAACTGTAAGATA | SEQ ID NO: 645 |
| MLT1E | TTGTGAGACCCTGAAGCAGAGGACCCAGCTAAGCTGTGCCCGGACTCCTG | SEQ ID NO: 646 |
| MLT1F | CATCTTGACTGCAACCTCATGAGAGACCCTGAGCCAGAACCACCCAGCTA | SEQ ID NO: 647 |
| MLT2A1 | GTTCTTCAGTTTTGGGACTCGGACTGGCTCTCCTTGCTCCTCAGCTTGCA | SEQ ID NO: 648 |
| MLT2B2 | TCACGTGAGCCAATTCCCCTAATAAATCYCYTCTATCCATCCTATTGGTT | SEQ ID NO: 649 |
| MLT2C2 | CCACAATCGCGTGAGCCAATTCCTTAAAATAAATCTCTCTCTACACACAC | SEQ ID NO: 650 |
| MLT2D | TCTGCCTGCCTGATNGTCTTCGAACTGGAATATCAGCTCTGCGGATTTTG | SEQ ID NO: 651 |
| MER4A | TAAAASCAAGCTGTRCCCCGACCACCTTGGGCACATGTCGTCAGGACCTC | SEQ ID NO: 652 |
| MER4B | CTAAAATGTATAAAASCAAGCTGTRCCCCGACCACCTTGGGCACATGTKG | SEQ ID NO: 653 |
| MER4C | ATTGAAGCCCTCAAAATCATCTTTGGAGAAAGGCACAGACCACAGATGTT | SEQ ID NO: 654 |
| MER9 | GCTGTGAGACCCCTGATTCCCACTTCACACCTCTATATTTCGTGTGTGTG | SEQ ID NO: 655 |
| MER11A | CACGGTCCTACCGATATGTGATGTCACCCCYGGAGGCCCAGCTGTAAAAT | SEQ ID NO: 656 |
| MER11B | CCGGATRCCCAGCTTTAAAATTTCTCTCTTTTGTACTCTGTCCCTTTATT | SEQ ID NO: 657 |
| MER39 | GGTCTTTGGGTCTTCATTTCTGAAGGCTCCCATGTCACGTAAAACTTTGA | SEQ ID NO: 658 |
| MER48 | TGTTGTTGTGGACGCGCTCTCGGGGTTSGAACCGAYACAAGARCCTTACA | SEQ ID NO: 659 |
| LOR1 | TCTTCCTTGGCAATAMTYRTTGTCTCAGTGATTGGCTTTCTGTGCAGTGA | SEQ ID NO: 660 |
| MER49 | TGCGGGATGGCCACCTTGCAGGCTGTAACCCTTTATAAGAAATAAAGTCT | SEQ ID NO: 661 |
| MER39B | TGCCTTTTCTCCWATTAATCTGCCTTTTGTSAGTTGATTTTTCAGTGAAM | SEQ ID NO: 662 |
| MER61 | AAGCCTAAWTTTTCGTGGCCGTGTGACAAGGACCCCGTCTTTAGCTGAAC | SEQ ID NO: 663 |
| MER31 | CCTGTACCTATCGCAATGGTCCTGAATAAAGTCTGCCTTACCGTGCTTTA | SEQ ID NO: 664 |
| MER34 | GCCGGAAACTCTAAGAGGGTAGAGGWAAAATTTTTCCTTCYCTNCCATGG | SEQ ID NO: 665 |
| MER41C | TTTACACTGTGGAATCACCCTGAATTCTTTCTTGCATGAGATCCAAGAAC | SEQ ID NO: 666 |
| MER50 | TGCTCTAAAACTTGCCTCGGTCTCTTTTTCTGCCTTATGCCCCTCAGTCG | SEQ ID NO: 667 |
| MER65A | GAATATGCACATAGTTTACTATGGCACGCGTATTCCCATTGCAATGCTCT | SEQ ID NO: 668 |
| MER65B | GTGTATGCCCCAAATTGCAATTCTGTTCTTCACATGTTATTCCCAAATAA | SEQ ID NO: 669 |
| MER66A | AGCCGCTTCAATAAAAGTTGCTGTCTAATACCACCARCTCGCCCTTGAAT | SEQ ID NO: 670 |
| MER66B | GTGTATGCCCCAAATTGCAATTCTGTTCTTCACATGTTATTCCCAAATAA | SEQ ID NO: 671 |
| MER67A | ATTCTCCCTTTAAAACGCCCAGTCACCTCTGCACAAATCGAAGCTGAGCT | SEQ ID NO: 672 |
| MER67B | CCTCATTCTCCCTTTAAAACGCCCAGTCACCTCTGCACAAATTGGAATGG | SEQ ID NO: 673 |
| MER67C | TAGCAGATTGCCTGTGATGCGCATCACATTCTGGTTTAATGCTTATTCAA | SEQ ID NO: 674 |
| MER68A | CCTGTGAGTCCTCCTAGCGAATCACCGAACCTGGGGGTGGTCTTGGGAAC | SEQ ID NO: 675 |
| MER68B | TTCCCTTTGCTGATCTTGCCGTGTATCCTTACNRTGTCGCTGTAATAAAT | SEQ ID NO: 676 |
| MER70A | TGTTCTGTCTCACCGGACTCAGACAAGTTGGTAACCAGTGCACAGTGAAC | SEQ ID NO: 677 |
| MER70B | TCNGACCCCTATTCCTGGTGGTTGGCATAGTGATGATCTTTGCTATTCTC | SEQ ID NO: 678 |
| MER72 | GCTGCAACCCTTTATGAGAAATAAAGCTCTCCTTTCCAAATTTATGAACC | SEQ ID NO: 679 |
| MER73 | GGTGACGGGGTACGACTGGGTTTCAAACAACTTATGTCAGGCCTAAAAAT | SEQ ID NO: 680 |
| MER74 | AAGCATGATTAATACAAKYTGGTCTGTGATGAACGGATGCCAAATAGWCG | SEQ ID NO: 681 |
| MER76 | TGTTGCCTTAATCGGCTNCTCTGACACCCGGCAGCTCAGCTCTCTCTCCA | SEQ ID NO: 682 |
| MER77 | CTTCTAGCGAATCACTGAACCTGAGGGTGGTCTTGGGGACCCCCGACACA | SEQ ID NO: 683 |
| MLT1G | GCGTCTTGACTGCGCCGATACCACGTGGGACAGAGAWGAACTRCCCAGCT | SEQ ID NO: 884 |
| PABL_A | AATAAAAACTCTCTTCCTCCCCAGTTCATCTGCATCTCGTTATTGGGCCA | SEQ ID NO: 685 |
| PABL_B | CCAGTTCATCTGCATCTCGTTATTGGGCCACGAGAATAAGCAGCCCGACC | SEQ ID NO: 686 |
| MER41D | ATAAACTTGCTCTTCTCACTGTACTCCGCAACTCGCCTTGAATTCCTTCC | SEQ ID NO: 687 |
| MER51A | CTCTGCTTTTGTTGCTTCATTCTTTCCTTGCTTTGTTTGTGCGTTTTGTC | SEQ ID NO: 688 |
| MER51B | CTCTGCTTTTGTTGCTTCATTCTTTCCTTGCTTTGTTTGTGCGTTTTGTC | SEQ ID NO: 689 |
| MER57A | ATCTTCTACCACATGGCTGCACTGGAGTCTCTGAACCTACTCTGGTTCTG | SEQ ID NO: 690 |
| MER57B | TATAAATTTGTTCCGACCACGAGGCATCCCTGGAGTCTCTCTGAATCTGC | SEQ ID NO: 691 |
| MER65C | ACCTCCAACCTTCTCTTTGTTC1TTGGACATACCGAAGACCACCTGGTCT | SEQ ID NO: 692 |
| MER83 | ACAACTGTCTTGGTAAATTATTTTTACCTCCCGCGCCACCGGCCCCAGAT | SEQ ID NO: 693 |
| MER54 | TGAAAGATACACTGTAAACACCCACAACCAMCTTCCCTGGAGCCCCATCA | SEQ ID NO: 694 |
| MER87 | ACTTACTGGCTGTCGWGCGGTGAGCAGTACCAGCTTTGGATTCAGTTACA | SEQ ID NO: 695 |
| MER74A | AATGGCAGTCGTCTCCTGATCTGTTGGCCTTACCATACCTGAATAATAAT | SEQ ID NO: 696 |
| MER74B | CTTTTCAATGGCAGTCGTCTCCTGATCTGTTGGCCTTACCATACCTSAAT | SEQ ID NO: 697 |
| MER88 | AGGGGAACTTGTGGCAGGGACCAGCCTTATCACACTGGTGCACCTGGTCA | SEQ ID NO: 698 |
| MER54B | AGCCATTTGGGTGTGGTGTAGAACTGGAAACTGTGTCAAGGGTGACTGAG | SEQ ID NO: 699 |
| MER31A | AAATTCCCACTTGCCCATGCTGTATTCGGAGTTGAGCCCAATCTCTCTCC | SEQ ID NO: 700 |
| MER31B | TCCCCACTTGTCCTTGCTGTATTCGGAGTTGAGCCCAATCTCTCTCCCCT | SEQ ID NO: 701 |
| MER67D | ATCCACCTGCCTTTTGTTTCAGNGGAGTTGAGTTCAANCTCTAACCCCTA | SEQ ID NO: 702 |
| MER11C | TTGTACTCTGTCCCTTTATTTCTCAAGCCAGCCGACGCTTAGGGAAAATA | SEQ ID NO: 703 |
| MER11D | ACTATCTTGTGTGTGTCTATTATTTCTCAACCTGCCGATCCGCCTAGGAG | SEQ ID NO: 704 |
| MER61B | CGCCCAATAAATTCTGCTCCTCACCCTTCAATGTGTCCGCGWGCCTAATC | SEQ ID NO: 705 |
| MER61C | GKGACAAGAACCCGGGTTTTAGCTGAACTAAGGAGCAAAATYCTGCAWCA | SEQ ID NO: 706 |
| MER92A | GTTCCTGAGGTCGGAGCGTTCTCCCTATTGCAATAGTCTTTTTGAATAAA | SEQ ID NO: 707 |
| MER92B | TTCTGCCTGAACTTTGAGATGCTTGCAGATCTTATGGTCAGAGCGTTCTC | SEQ ID NO: 708 |
| MER92C | TATCTACCCCTTCCTATAAAAGTCCAAGGCAAAACCACCCTGCCGAGACA | SEQ ID NO: 709 |
| MER93 | CTTCCTCATNCACCYTATAAAAGCCTTTCCTTCAAGCCCCTCCGGCGGAG | SEQ ID NO: 710 |
| MLT1H | CACAGATGCATGAGGGAGCCCAGCCGAGACCAGAAGAACCACCCAGCTGA | SEQ ID NO: 711 |
| MER89 | AAGCTCTGAATAAATAGCCTTTGCTTGTTCTCATTTGGKTGGTCTTCATT | SEQ ID NO: 712 |
| MER90 | CCTCGCTGCARCGAGCAATAAACCCAACTTGTTCAACCACAGGTGTGTTC | SEQ ID NO: 713 |
| MLT2A2 | TGTGGGACTTCACCTTGTGATCGTGTGAGTCAATACTCCTTAATAAACTC | SEQ ID NO: 714 |
| MLT1I | GAGCAGAGCCCCAGCCGACCCGCGATGGACATGTAGCATGAGCAAGAAAT | SEQ ID NO: 715 |
| MER52B | GCCACAGAGGTTTCCGGCCAGAAAAGCGACACCCCAAGGATCCCATGACA | SEQ ID NO: 716 |
| MER52C | ACACTAAATAAAGCTCTTCTTCGTCTTCTTCACCCTTCACTTGTCTGCGT | SEQ ID NO: 717 |
| MER95 | TTGARGTCTCCCGGTTCGCGARCTGTWCTTTCTCTYATTGTATGCACAAT | SEQ ID NO: 718 |
| MLT1J | ATGGAGCAGAGCTGCCATACCAGCCCTGGACTGCCTACCTCTAGACTTCT | SEQ ID NO: 719 |
| MLT1K | AGCTACCCCTGGACTTTTCAGTTACGTGAACCAATAAATTCCCTTTTTTG | SEQ ID NO: 720 |
| MER101 | TTCGTTTTACACCGAAGGCTGCATCTCCCCGGTTTGCAAACTGTTCACTG | SEQ ID NO: 721 |
| MER41E | TTTCTGACTCATCCTTGAATTCCTTCTCGCGATGGTGTCAAGAGCCTGGA | SEQ ID NO: 722 |
| MLT2E | TCCCCCCTCCAGACCTTCACTTCCCCAGCTCCTCCCACAATTGTATAAGG | SEQ ID NO: 723 |
| MLT1E1 | TGATTTCAGCCTTGTGAGACCCTGAGCAGAGGACCCAGCTAAGCCGTGCC | SEQ ID NO: 724 |
| MLT1J1 | AGCCACTGTACATTTTGGGGTTTATTTGTTACAGCAGCTAGCGTTACCTT | SEQ ID NO: 725 |
| MLT1J2 | CCTGAGTCACTACNTGGAGGAGAGCCACCCACACCCGACCAGAACCCNCA | SEQ ID NO: 726 |
| MLT1E2 | TTGATTTCGGCCTTGTGAGACCCTGAGCAGAGAACCCAGCCGAGCCCACC | SEQ ID NO: 727 |
| MLT1G1 | TGCCCAAATTGCAGATTCGTGAGCAAAATAAATGATTGTTGTTGTTTTAA | SEQ ID NO: 728 |
| MER110 | CTCAGCTTTGCTTGATCAACAGGTTTTNTTTTCTGGTGGTCTTTTTGGGG | SEQ ID NO: 729 |
| MER110A | TGGTGCTCYCCCTTACCACAGTAAGCAATAAACTCAGCTTTGTCTTATCA | SEQ ID NO: 730 |
| MLT1F1 | GAGAGACCCTGAGCCAGAACCACCCAGCTAAGCTGCTCCCGAATTCCTGA | SEQ ID NO: 731 |
| MER101B | GGCTGTGTCTCCCTGGTTTGCAAACTGTTCACTGGAATAAACTCTCCTCC | SEQ ID NO: 732 |
| MLT1G2 | CCCTGCTGTGCCCTGTCCGAATTCCTGACCCACAGAATCCGTGAGCATAA | SEQ ID NO: 733 |
| MSTA1 | AGATGCTCGCACCATGCTTTTTGTCCAGCCAGCAGAAYTATGAGCCAAAT | SEQ ID NO: 734 |
| MLT1G3 | AGCCTTCAAGTCTTCCCAGCTGAGGCCCCAGACATCATGGAGCAGAGAGC | SEQ ID NO: 735 |
| MSTA2 | TGCCCTTGAACTTCCCAGCCTGCAGAACCATGAGCTAAATAAACCTCTTT | SEQ ID NO: 736 |
| MLT1C1 | GCCTCCAGAGGGAGCATGGCCCTGCTGACACCTTKGATTTCAGCCCAGTG | SEQ ID NO: 737 |
| MSTD | GATGACGCAGCAAGAAGGCCCTCACCAGATGCCGGCNCCWTGATCTTGGA | SEQ ID NO: 738 |
| MER51C | TCTCGCTTTAATAAATTCCTGCTTTCGCTGCTTCGTTCCTGTGTTTCATT | SEQ ID NO: 739 |
| MER21A | TGGTGTGAGAGCAGAGGAAAAACACGGTTTGAGAGAGTTTTCCCGAAACA | SEQ ID NO: 740 |
| MER34B | TCTGTCTTTTGTTACAGGGGTCTATTCCAACTAAGAACTTATGAGGGTTG | SEQ ID NO: 741 |
| MER54A | TATCTGGATCGACCACATTGAGGAACTGGGAGGAGGCGGAGAACTGGAAA | SEQ ID NO: 742 |
| MER74C | GCCTTTCATCTATCCGAGTGTCANTGTGTTGTGTCCCGCCATCAAAAGAA | SEQ ID NO: 743 |
| THE1A | CTCATTTTCCTCTTGCCGCCGCCATGTAAGAAGTGCCTTTCGCCTCCCGC | SEQ ID NO: 744 |
| THE1C | ATGTGAAGAAGGACGTGTTTGCTTCCCCTTCCGCCATGATTGTAAGTTTC | SEQ ID NO: 745 |
| MSTB | ATGATTGNAAGCTTCCTGAGGCCTCACCAGAAGCCGAGCAGATGCCGGCG | SEQ ID NO: 746 |
| MSTB1 | GCCATGCTTCTTGTACAGCCTGCAGAACCGTGAGCCAAATAAACCTCTTT | SEQ ID NO: 747 |
| MER51E | CTGTGGAGTGTACTTTCGCTTCAATAAATCTGTGCTTTCGTTACTNCGTT | SEQ ID NO: 748 |
| MER41F | TGGGTGGCACCACAGTTCCGAGAAATCTTCACCTTTTTCCAGGAATCTTC | SEQ ID NO: 749 |
| MER65D | TAAAAGCTTCCCTTTACCCTCCCCTCTTCAGATGCATCTGTGGCTTGCCA | SEQ ID NO: 750 |
| MER72B | TCCTTTTACCCCTCCCTCAAAGTGCTTTGCTCTCAGCTTCTGCCAGAGGC | SEQ ID NO: 751 |
| MER34C | TTGTTACAGGGGTCTGTCCCAGCTAAGAACTATGAAGGGTAGAGAGAAAA | SEQ ID NO: 752 |
| MER50B | GATATGCCGCYGGTAACTCAGGGTAACTCGGATCTCTTCCACCGGTAACA | SEQ ID NO: 753 |
| MER93B | CTATAAAAGCCTCCCCCTTGCATTCCCTCGGTGGAGCTCCCGAACCACTT | SEQ ID NO: 754 |
| MLT1A1 | CATCTTGGAAGCAGAGASCAGGCCCTCACCAGACACCAAACCTGCTGGNA | SEQ ID NO: 755 |
| MLT1E1A | CTTGTGAGACCCTGAGCAGAGGACCCAGCTAAGCTGTGCCCAGACTCCTG | SEQ ID NO: 756 |
| MER4E1 | TCACGGGCCATGGTCACTCATATTTGGCTCAGAATAAATCTCTTCAAATA | SEQ ID NO: 757 |
| PRIMA4_LTR | TTTAAATTTGGAGCCCTCAAAATCATCTTCGGAGAAAGGCATAGACCTGT | SEQ ID NO: 758 |
| MLT1F2 | ACACCTTGATTGCAGCCTTGTGAGAGACCCTGAGCCAGAAGACCCAACTA | SEQ ID NO: 759 |
| MLT2B3 | CTTCTCAGCCTCCATAATCAAGTGAGCCAATTCCCCTAATAAATCCCTTC | SEQ ID NO: 760 |
| MER66C | GAGCAGTACCGTTCAATAAAAGATTGCTGTCTAACACCACTGGCTCACCC | SEQ ID NO: 761 |
| MER52D | CTCAGGCAAAGGHACCACHGGHCACAGAGGTTTCTGGCCAGAAAAGBGAC | SEQ ID NO: 762 |
| MER41G | TGCTTTGCAATAAAAGCTTCTTGCCTTTCGCTTCATTCTGACTCATCCCT | SEQ ID NO: 763 |
| MER21C | TGTGGGATCTGATGCTAACTCCAGGGTAGATAGTGTCAGAATTGAATTAA | SEQ ID NO: 764 |
| MLT2B4 | CCTGGGTCTCCAGCTTGCCAACTCACCCTGCAGATCTTGGGACTTCTCAG | SEQ ID NO: 765 |
| MER9B | TAAATATGTGGGTCAAACTCTGTTTGTGGCTCTCAGCTCTGAAGGCTGTT | SEQ ID NO: 766 |
| MLT1H2 | TACACCATGTGGAGCAGAAGAACCACCCAGCTGAGCCCAGCCAACACAGA | SEQ ID NO: 767 |
| MER4A1 | AAAACCAAGCTGTGCTCTGACCACCTTGGGCACATGTCGTCAGGACCTCC | SEQ ID NO: 768 |
| MER4D1 | TCANAGGCCATGGTCACTCATATTTGGCTCAGAATAAATCTCTTCAAATA | SEQ ID NO: 769 |
| THE1D | TGCTTGCTTCCCCTTTGCCTTCTGCCATGATTGTAAGTTTCCTGAGGCCT | SEQ ID NO: 770 |

The expression and methylation patterns of the present invention can be evaluated by utilizing high-density arrays or microarrays. As defined herein, “microarray” can be a chip, a glass slide or a nylon membrane comprising different types of material, such as, but not limited to, nucleic acids, proteins or tissue sections. By utilizing microarray technology, a plurality of transposable element sequences from transposable element families can be analyzed simultaneously to obtain expression and/or methylation patterns. One of skill in the art can design a microarray chip or glass slide that contains the representative nucleic acid sequences of all of the members of a particular transposable element family or the nucleic acid sequences of select members of a particular transposable element family. A chip can also contain the nucleic acid sequences of selected transposable elements from one or more families. Array design will vary depending on the transposable element families and the sequences from these families being analyzed. One of skill in the art will know how to design or select a chip that contains the transposable element sequences associated with a cell at a particular stage of pluripotency. Such microarray chips can be obtained from commercial sources such as Affymetrix, or the microarray chips can be synthesized. Methods for synthesizing such chips containing nucleic acid sequences are known in the art. See, for example, U.S. Pat. No. 6,423,552, U.S. Pat. No. 6,355,432 and U.S. Pat. No. 6,420,169 which are hereby incorporated in their entireties by this reference.

The present invention also provides microarray slides or chips comprising transposable element sequences or fragments thereof from transposable element families.

As stated above, a microarray slide or chip can contain the representative nucleic acid sequences of all of the members of one or more transposable element families or the nucleic acid sequences of select members of one or more transposable element families. The present invention also provides for a kit comprising a microarray slide or chip of the present invention for determining the stage of pluripotency of a cell. Utilizing the methods of the present invention, a chip(s) or glass slide(s) that specifically detect a cell's stage or type of pluripotency can be synthesized. For example, if it is known that transposable element sequences from fifty families are expressed in a fully pluripotent stem cell, a chip that contains the necessary transposable element sequences from these fifty families can be synthesized, such that one of skill in the art can utilize a kit, containing this chip, for detecting and staging fully pluripotent stem cells. Similarly, utilizing the expression patterns of transposable element sequences characteristic of cells that are partially pluripotent (e.g., capable of differentiating into a type of brain or neural cell but not into liver cells), it is possible to manufacture a kit containing a chip comprising the transposable element sequences in order to diagnose and stage cells possessing this degree of developmental potential.

Microarray techniques would be known to one of skill in the art. For example, U.S. Pat. No. 6,410,229 and U.S. Pat. No. 6,344,316, both hereby incorporated by this reference, describe methods of monitoring expression by hybridization to high density nucleic acid arrays. For example, one skilled in the art would first produce fluorescent-labeled cDNAs from mRNAs isolated from stem cells. A mixture of the labeled cDNAs from the stem cells is added to an array of oligonucleotides representing a plurality of known transposable elements, as described above, under conditions that result in hybridization of the cDNA to complementary-sequence oligonucleotides in the array. The array is then examined by fluorescence under fluorescence excitation conditions in which transposable element polynucleotides in the array that are hybridized to cDNAs derived from the stem cells can be detected and quantified.

The expression patterns of the present invention can also be determined by assaying for mRNA transcribed from transposable elements, in situ hybridization and Northern blotting and assaying for proteins expressed from a mRNA. Particular protein products translated from mRNAs transcribed by transposable element genes can be detected by utilizing immunohistochemical techniques, ELISA, 2-D gels, mass spectrometry, Western blotting, and enzyme assays.

In the present invention, patterns of expression can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family are being analyzed. For example, the present invention provides for the determination of an expression pattern of one family of transposable elements in which one, two, three, four, five, ten, fifteen, twenty, twenty five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of a transposable element family are analyzed. The present invention also provides for the determination of an expression pattern of two families, wherein one, two, three, four, five, ten, fifteen, twenty, twenty five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an expression pattern of three families, wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an expression pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference expression pattern can be obtained for fully pluripotent stem cells, as well as for cells that have a lesser degree of developmental potential (reduced pluripotency). Therefore, the present invention provides a method of assigning an expression pattern of transposable elements to a fully pluripotent stem cell comprising: a) determining expression of one or more families of transposable elements in a fully pluripotent stem cell and assigning the expression pattern obtained from step a) to the cell.

The present invention also provides a method of assigning an expression pattern of transposable elements to a pluripotent stem cell comprising: a) determining expression of one or more families of transposable elements in a pluripotent stem cell and assigning the expression pattern obtained from step a) to the cell.

Also provided by the present invention is a method of assigning an expression pattern of transposable elements to a differentiated cell comprising: a) determining expression of one or more families of transposable elements in a differentiated cell and assigning the expression pattern obtained from step a) to the cell.

The present invention also provides a method of determining the developmental potential of a cell comprising: a) determining expression of one or more families of transposable elements in a cell to obtain an expression pattern; b) matching the expression pattern of step a) with a known expression pattern for a cell and c) determining the level of developmental potential of a cell based on matching of the expression pattern of a) with a known expression pattern for a cell with a specific level of developmental potential.

In the methods of the present invention, the expression pattern obtained from a sample of cells taken from a subject can be obtained from outside sources, such as a testing laboratory or a commercial source. Therefore, the step of obtaining the expression pattern can be performed by one skilled artisan and the step of comparing the expression pattern can be performed by a second skilled artisan. Thus, the present invention provides a method of determining the developmental potential of a cell comprising a) matching a test transposable element expression pattern of a cell with a known expression pattern for a cell at a specific stage of developmental potential; and b) determining the developmental potential of a cell based on matching of the test expression pattern of a cell with a known expression pattern for a cell at a specific stage of developmental potential.

For example, one of skill in the art can obtain a fertilized oocyte derived pluripotent stem cell and determine the expression pattern of one or more transposable element families. By determining which transposable elemnt families are expressed as well as which members of these transposable element families are expressed, one of skill in the art can assign this pattern to a fertilized oocyte derived pluripotent stem cell. This can be done for another stem cell with a more limited developmental potential than a fertilized oocyte, for example, a stem cell derived from a brain, such that a library of expression patterns are readily available not only to identify a cell with fully pluripotent or pluripotent potential but to determine the stage of pluripotency, i.e., level of developmental potential. Similarly, this can be done for stem cells derived from any tissue, or for oocytes in which a nucleus derived from a differentiated cell has been introduced to determine the degree to which that nucleus has reacquired pluripotency. By determining the expression patterns of transposable elements in cells with different stages of pluripotency, the skilled artisan can determine which transposable element families and which members of these families are markers of the developmental potential of cells.

Such libraries of expression patterns are useful for determining the developmental potential of stem cells. For example, a nucleus from a fully differentiated cell from a patient with Parkinson's disease can be transplanted into an enucleated oocyte. Once the expression patterns of putative stem cells descendent from this oocyte are determined according to the methods of the present invention, this expression pattern can be compared to a library of expression patterns to determine the level of pluripotency associated with the expression pattern. Once this is determined, a decision can be made with regard to the potential of these stem cells to regenerate appropriate neural cells if implanted in the patient's brain. The present methods will also be useful in evaluating the effectiveness of various treatments in stimulating stem cells to develop or, conversely, to monitor the effectiveness of treatments to stimulate determined and/or differentiated cells to regain pluripotency. For example, a sample of partially or fully differentiated neural cells could be treated in vitro with oocyte cellular extracts or other chemicals, small molecules, peptides, growth factors, etc. designed to reprogram differentiated cells to regain full or partial pluripotency. Expression patterns can be obtained from these treated cells and compared to expression patterns pre-established to be characteristic of pluripotent stem cells. Since the skilled artisan will have reference patterns for the fully differentiated cell, as well- as, reference patterns for a a fully pluripotent stem cell and stem cells of more limited pluripotency, changes in transposable element expression after treatment can be monitored to determine if the treatment results in a transposable element expression pattern which more closely resembles a fully pluripotent or pluripotent stem cell.

For example, if before treatment, certain families and members of these families are expressed, and after treatment, more families and/or members of these families are expressed, it can be said that this particular treatment is effective in increasing the developmental potential of the cell or in reprogramming the differentiated cell to become pluripotent. In some instances, effective treatments may involve decreasing the expression of certain transposable elements and increasing the expression of others. Therefore, once libraries of expression patterns are established from untreated differentiated cells, one of skill in the art will know whether or not treatment is effective in a particular cell lineage by comparing the expression pattern of a sample from samples of cells at different stages of treatment, with reference patterns established for the fully pluripotent stem cells. If a treatment is not successful in a particular cell lineage, the skilled artisan will recognize this by noting that the expression pattern is not changing as expected, and other dosages, or treatments can be employed.

Therefore, the present invention also provides a method of identifying a factor that increases the developmental potential of a cell comprising: a) determining expression of one or more families of transposable elements, in a cell to obtain a first expression pattern; b) administering a putative factor that increases developmental potential to the cells; c) determining expression of one or more families of transposable elements in a cell after administration of the factor to obtain a second expression pattern; and d) comparing the second expression pattern with the first expression pattern such that if the differences between the expression patterns can be correlated with an increase in developmental potential, the factor increases the developmental potential of the cell. The changes observed between expression patterns can vary depending on the type of differentiated cell.

In some instances, effective treatment of a cell, i.e., increasing the developmental potential of a cell, will result in fewer transposable elements being expressed in the second expression pattern as compared to the first expression pattern. In other instances, there may be more transposable elements expressed in the second expression pattern as compared to the first expression pattern.

The expression patterns of the present invention can also be used in combination with other diagnostic markers of genomic reprogramming, such as the loss of expression of genes known to be characteristically and specifically expressed in specific types of differentiated cells. The expression patterns of the present invention can also be used with methylation patterns and/or chromatin status patterns to assess the developmental potential of any type of cell.

Analysis of Methylation Patterns

The present invention also provides methods of assessing methylation status of transposable element sequences and its role in development. Thus, also provided by the present invention is a method of determining a methylation pattern of one or more families of transposable elements in a cell comprising determining methylation of one or more families of retroviral elements. By analyzing global methylation patterns of transposable elements, one of skill in the art can assign particular methylation patterns to the various stages of developmental potential of a cell. These methylation patterns can be utilized with the expression patterns and chromatin status patterns described herein to assess the developmental potential of a cell or cells.

In the present invention, methylation patterns can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family. For example, the present invention provides for the determination of a methylation pattern of one family of transposable elements in which one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of the transposable element family are analyzed. The present invention also provides for the determination of a methylation pattern of two families, wherein one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a methylation pattern of three families, wherein one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an methylation pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference methylation pattern can be obtained for fully pluripotent stem cells, as well as for. cells that have more limited developmental potential (reduced pluripotency). Therefore, the present invention provides a method of assigning a methylation pattern of transposable elements to a fully pluripotent stem cell comprising: a) determining methylation of one or more families of transposable elements in a fully pluripotent stem cell and assigning the expression pattern obtained from step a) to the cell.

The present invention also provides a method of assigning a methylation pattern of transposable elements to a pluripotent stem cell comprising: a) determining methylation of one or more families of transposable elements in a pluripotent stem cell and assigning the methylation pattern obtained from step a) to the cell.

Also provided by the present invention is a method of assigning a methylation pattern of transposable elements to a differentiated cell comprising: a) determining methylation of one or more families of transposable elements in a differentiated cell and assigning the methylation pattern obtained from step a) to the cell.

The present invention also provides a method of determining the developmental potential of a cell comprising: a) determining methylation of one or more families of transposable elements in a cell to obtain a methylation pattern; b) matching the methylation pattern of step a) with a known methylation pattern for a cell and c) determining the level of developmental potential of a cell based on matching of the expression pattern of a) with a known methylation pattern for a cell with a specific level of developmental potential.

In the methods of the present invention, the methylation pattern obtained from a sample cell taken from a subject can be obtained from outside sources, such as a testing laboratory or a commercial source. Therefore, the step of obtaining the methylation pattern can be performed by one skilled artisan and the step of comparing the methylation pattern can be performed by a second skilled artisan. Thus, the present invention provides a method of establishing the developmental potential of a cell or cells comprising: a), matching a test transposable element methylation pattern of a cell with a known methylation pattern for a cell with a specific level of developmental potential; and b) determining the level of developmental potential of the cell based on matching of the test methylation pattern with a known methylation pattern for a cell with a specific level of developmental potential.

For example, one of skill in the art can obtain a fertilized oocyte derived pluripotent stem cell and determine the methylation pattern of one or more transposable element families. By determining which transposable element families are methylated as well as which members of these transposable element families are methylated, one of skill in the art can assign this pattern to a fertilized oocyte derived pluripotent stem cell. This can be done for another stem cell with a more limited developmental potential than a fertilized oocyte, for example, a stem cell derived from a brain, such that a library of methylation patterns are readily available to not only to identify a cell with pluripotent potential but to determine the stage of pluripotency, i.e., level of developmental potential. Similarly, this can be done for stem cells derived from any tissue, or for oocytes in which a nucleus derived from a differentiated cell has been introduced to determine the degree to which that nucleus has reacquired pluripotency. By determining the methylation patterns of retrolements in cells with different stages of pluripotency, the skilled artisan can determine which transposable element families and which members of these families are markers of the level of pluripotency and developmental potential of cells.

Such libraries of methylation patterns are useful for determining the developmental potential of stem cells. For example, a nucleus from a filly differentiated cell from a patient with Parkinson's disease can be transplanted into an enucleated oocyte. Once the methylation pattern of putative stem cells descendent from this oocyte is determined according to the methods of the present invention, this methylation pattern can be compared to a library of methylation patterns to determine the level of pluripotency associated with the methylation pattern. Once this is determined, a decision can be made with regard to the potential of these stem cells to regenerate appropriate neural cells if implanted in the patient's brain. The present methods will also be useful in evaluating the effectiveness of various treatments in stimulating stem cells to develop or, conversely, to monitor the effectiveness of treatments to stimulate determined and/or differentiated cells to regain pluripotency. For example, a sample of partially or fully differentiated neural cells could be treated in vitro with oocyte cellular extracts or other chemicals, small molecules, peptides, growth factors etc. designed to reprogram differentiated cells or to increase pluripotency. Methylation patterns can be obtained from these treated cells and compared to methylation patterns pre-established to be characteristic of pluripotent stem cells. Since the skilled artisan will have reference patterns for the fully differentiated cell, as well as a fully pluripotent stem cell and stem cells of more limited pluripotency, changes in transposable element methylation after treatment can be monitored to determine if the treatment results in a transposable element methylation pattern that more closely resembles the methylation pattern for a pluripotent stem cell.

For example, if before treatment, certain families and members of these families are methylated, and after treatment, fewer families and/or members of these families are methylated, it can be said that this particular treatment is effective in increasing the developmental potential of the cell or in reprogramming the differentiated cell to become pluripotent. In some instances, effective treatments may involve decreasing the methylation of certain transposable elements and increasing the methylation of others. Therefore, once libraries of methylation patterns are established from untreated differentiated cells, one of skill in the art will know whether or not treatment is effective in a particular cell lineage by comparing the methylation pattern of a sample from samples of cells at different stages of treatment, with reference patterns established for the fully pluripotent stem cells. If a treatment is not successful in a particular cell lineage, the skilled artisan will recognize this by noting that the methylation pattern is not changing as expected, and other dosages, or treatments can be employed.

Therefore, the present invention also provides a method of identifying a factor that increases the developmental potential of a cell comprising: a) determining methylation of one or more families of transposable elements in a cell to obtain a first methylation pattern; b) administering a putative factor that increases developmental potential to the cells; c) determining methylation of one or more families of transposable elements in the cell after administration of the factor to obtain a second expression pattern; and d) comparing the second methylation pattern with the first methylation pattern such that if the differences between the methylation patterns can be correlated with an increase in developmental potential, the factor increases the developmental potential of the cell. The changes observed between expression patterns can vary depending on the type of differentiated cell.

In some instances, an effective treatment will result in fewer transposable elements being methylated in the second methylation pattern as compared to the first methylation pattern. In other instances, there may be more transposable elements methylated in the second pattern as compared to the first methylation pattern.

The methylation patterns of the present invention can also be used in combination with other diagnostic markers of genomic reprogramming, such as the loss of methylation of genes known to be characteristically and specifically expressed in specific types of differentiated cells (e.g the differentiated liver cell marker DDP IV-dipeptidyl peptidase-see Oh et al. 2000 Hepatocyte growth factor induces differentiation of adult rat bone marrow cells into a hepatocyte lineage in vitro. Biochem. Biophys. Res. Commun. 279: 500-504).

Methods of measuring methylation are known in the art and include, but are not limited to methylation-specific. PCR, methylation microarray analysis, use of a methyly binding column and ChIP (a chromatin immunoprecipitation approach) analysis. Methylation can also be monitored by digestion of nucleic acid sequences with methylation sensitive and non-sensitive restriction enzymes followed by Southern blotting or PCR analysis of the restriction products (See Takai et al. “Hypomethylation of LINE1 retrotransposon in human hepatocellular carcinomas, but not in surrounding liver cirrhosis” Jpn J. Clin. Oncol. 30(7) 306-309). One of skill in the art could also utilize methods in which genomic DNA is digested followed by PCR. (See, for example, Cartwright et al., “Analysis of Drosophila chromatin structure in vivo” Methods in Enzymology, Vol. 304)

Methylation-specific PCR (MSP) technology utilizes the fact that DNA in humans is methylated mainly at certain cytosines located 5′ to guanosine. This occurs especially in GC-rich regions, known as CpG islands. To distinguish the methylation state of a sequence, MSP relies on differential chemical modification of cytosine residues in DNA. Treament with sodium bisulfite converts unmethylated cytosine residues into uracil, leaving the methylated cytosines unchanged. This modification thus creates different DNA sequences for methylated and unmethylated DNA. PCR primers can then be designed so as to distinguish between these different sequences. Two sets of primers (and additional control sets of primers) are designed: one set with sequences annealing to unchanged (methylated in the genomic DNA) cytosines and the other set with sequences annealing to the altered (unmethylated in the genomic DNA) cytosines. A comparison of PCR results using the two sets of primers reveals the methylation state of a PCR product. If the primer set with the altered sequence gives a PCR product, then the indicated cytosine was unmethylated. If the primer set with the unchanged sequence gives a PCR product, then the cytosines were methylated and thus protected from alteration. Evron et al. (“Detection of breast cancer cells in ductal lavage fluid by methylation-specific PCR,” Lancet 2001, 357: 1335-1336) describes the use of MSP to detect breast cancer and is hereby incorporated in its entirety by this reference.

To use a microarray to study transposable element methylation, one of skill in the art would select for methylated and unmethylated DNA from total genomic DNA. The selectively isolated DNA is then hybridized to the transposable element array either directly or after amplification and patterns are compared between various cell types/tissue types as described earlier in the patent application.

There are several approaches for selecting methylated DNA. One method is chromatin immunoprecipitation (ChIP). Another method utilizes a column binding approach and a third option involves ligation of adapters to fragmented genomic DNA and methylation-specific restriction digestion of the ligation products followed by PCR amplification.

In all cases, the selected DNA fragments are labeled by incorporation of dNTPs coupled with fluorescent dyes (for example Cy3 or Cy5 coupled dNTPs) and hybridization to the microarray is performed according to standard protocols. One of skill in the art could utilize the BioPrime DNA labeling system from Life Technologies or other kits available for such labeling.

As stated above, microarray techniques would be known to one of skill in the art. For example, U.S. Pat. No. 6,410,229 and U.S. Pat. No. 6,344,316, both hereby incorporated by this reference, describe methods of hybridizing nucleic acids to high density nucleic acid arrays. For example, one skilled in the art would first produce fluorescent-labeled DNA isolated from the tissue of interest. A batch of labeled genomic/amplified genomic DNAs representing either one sample or a mixture of two samples from the tissue sources of interest is added to an array of oligonucleotides representing a plurality of known transposable elements, as described above, under conditions that result in hybridization of the DNAs to complementary-sequence oligonucleotides in the array. The array is then examined by fluorescence under fluorescence excitation conditions in which transposable element oligonucleotides in the array that are hybridized to genomic/amplified genomic DNAs derived from the tissue of interest can be detected and quantified.

ChIP technology involves in vivo formaldehyde cross-linking of DNA and associated proteins in intact cells, followed by selective immunoprecipitation of protein-DNA complexes with specific antibodies. Such an approach allows detection of any protein at its in vivo binding site directly. In particular, proteins that are not bound directly to DNA or that depend on other proteins for binding activity in vivo can be analyzed by this method. Since methylation involves methylation complexes that involve numerous proteins which interact with DNA, by utilizing ChIP technology, methylation complexes can be cross-linked to transposable element sequences to which they are bound and then an antibody specific to one of the proteins (i.e, one of the proteins involved in the methylation complex, such as methyltransferase or a protein having a methyl binding site, for example, MBD1) can be utilized to immunoprecipitate the methylation complex-DNA bound sequence. The complex can then be chemically released and the transposable element sequence to which it was bound can be identified. For references describing ChIP technology, see Orlando (“Mapping chromosomal proteins in vivo by formaldehyde crosslinked-chromatin immunoprecipitation,” TIBS 2000, 25:99-104) and Kuo et al. (“In Vivo Cross-Linking and Immunoprecipitation for Studying Dynamic Protein:DNA Associations in a Chromatin Environment,” 1999, 19: 425-433) both of which are incorporated in their entireties by this reference.

Formaldehyde crosslinking followed by chromatin immunoprecipitation is reviewed in Orlando 2000. By addition of formaldehyde to live tissue/cells, DNA and nearby proteins are cross-linked in vivo, followed by sonication of the tissue/cell suspension. The DNA is fragmented in the process. Antibodies recognizing methyl-binding proteins are added and the immune complexes are collected, thereby precipitating methylated DNA with associated proteins. DNA without methyl-binding proteins will be collected from the supernatant. The cross-linking step is then reversed for both fractions, followed by a DNA purification step. The isolated DNA can be ligated to linker oligonucleotides and amplified by PCR. Fluorescence labeling and hybridization is then performed as described above.

The column binding approach is used to select for methylated DNA after genomic DNA extraction. The column contains methyl-CpG-binding proteins, for example the methyl-binding domain of rat MeCP2, covalently linked to a histidine tag, then attached to a Ni-agarose matrix. Fragmented genomic DNA (digested with restriction enzymes, for example MseI) is run through the column. The column retains DNA containing methylated cytosines, unmethylated DNA is collected from the flow-through. Retained methylated DNA is recovered from the column. (Cross, S. H., Charlton, J. A., Nan, X. and Bird, A. P. (1994) Purification of CpG islands using a methylated DNA binding column. Nat Genet., 6, 236-244 and Brock, Huang, Chen and Johnson (2001) A novel technique for the identification of CpG islands exhibiting altered methylation patterns (ICEAMP). Nucleic Acids Research, l vol.29, no.24). The isolated DNA can be ligated to linker oligonucleotides and amplified by PCR. Fluorescence labeling and hybridization is then performed as described above.

Linker ligation/Methylation-specific restriction/PCR can also be utilized. The methods of the present invention can utilize a modified version of DMH (Differential Methylation Hybridization) (References: Huang et al. ‘Methylation profiling of CpG islands in human breast cancer cells’ Human Molecular Genetics 1999, Vol.8, No.3 and Yan et al. ‘Dissecting complex epigenetic alterations in breast cancer using CpG island microarrays’ Cancer Research 2001, 61, 8375-8380). Genomic DNA is digested with MseI. Then, the ends of the resulting fragments are ligated to linker oligonucleotides. Ligated fragments undergo restriction digestion with methylation-sensitive enzymes BstUI and/or HpaII, followed by PCR amplification of undigested fragments. Fluorescence labeling and hybridization is then performed as described above.

A COT-1 subtractive hybridization step can be utilized at some point before labeling the DNA to separate out the highly repetitive sequences from the sample (See Craig et al. ‘Removal of repetitive sequences from FISH probes using PCR-assisted affinity chromatography’ Human Genetics 1997, Vol. 100, 472-476).

Another technique, methylation-specific oligonucleotide (MSO) microarray, uses bisulfite-modified DNA as a template for PCR amplification, resulting in conversion of unmethylated cytosine, but not methylated cytosine, into thymine within CpG islands of interest. The amplified product, therefore, may contain a pool of DNA fragments with altered nucleotide sequences due to differential methylation status. A test sample is hybridized to a set of olignonucleotide arrays that discriminate between methylated and unmethylated cytosine at specific nucleotide positions, and quantitative differences in hybridization are determined by fluorescence analysis. For examples of methylation microarray techniques see Gitan et al. (“Methylation-specific oligonucleotide microarray: a new potential for high-throughput methylation analysis,” Genome Res. 2002, 12: 158-164.), Shi et al. (“Oligonucleotide-based microarray for DNA methylation analysis: Principles and applications,” J. Cell Biochem. 2003, 88: 138-143.), Yan et al. (“Applications of CpG island microarrays for high-throughput analysis of DNA methylation,” J. Nutr. 2002, 132: 2430S-2434S), Wei et al. (“Methylation microarray analysis of late-stage ovarian carcinomas distinguishes progression-free survival in patients and identifies candidate epigenetic markers,” Clin Cancer Res. 2002, 8: 2246-2252.), all of which are incorporated herein, in their entireties, by this reference.

Analysis of Chromatin Status

The present invention also provides methods of assessing the chromatin status of transposable element sequences and its role in the developmental potential of cells. These chromatin status patterns can be used in combination with transposable element expression patterns and/or methylation patterns described herein to assess the developmental potential of cells. One of the skill in the art would know how to assess chromatin status by methods standard in the art. See Orlando (“Mapping chromosomal proteins in vivo by formaldehyde crosslinked-chromatin immunoprecipitation,” TIBS 2000, 25:99-104) and Kuo et al. (“In Vivo Cross-Linking and Immunoprecipitation for Studying Dynamic Protein:DNA Associations in a Chromatin Environment,” 1999, 19: 425-433) both of which are incorporated in their entireties by this reference.

Thus, the present invention provides a method of assigning a chromatin status pattern of transposable elements to the level of developmental potential of a cell comprising: a) determining chromatin status of one or more families of transposable elements; and b) assigning the chromatin status pattern obtained from step a) to the level of developmental potential of a cell.

As utilized herein, “chromatin status” refers to the chromosomal structure or the chromosomal accessibility or the ability of restriction enzymes to access a transposable element sequence or a fragment thereof. Therefore, chromatin status patterns can contain sequences that are accessible to restriction enzymes and sequences that are not accessible to restriction enzymes.

The present invention also provides a method of determining the developmental potential of a stem cell comprising: a) determining chromatin status of one or more families of transposable elements in a stem cell to obtain a chromatin status pattern; b) matching the chromatin status pattern of step a) with a known chromatin status pattern for a cell at different stages of developmental potential ranging from a filly pluripotent stem cell to a fully differentiated cell and; c) determining the developmental potential of the stem cell based on matching the chromatin status pattern of a) with a known chromatin status pattern for a cell at a specific developmental stage.

In the present invention, chromatin status patterns can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family. For example, the present invention provides for the determination of a chromatin status pattern of one family of transposable elements in which one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of the transposable element family are analyzed. The present invention also provides for the determination of a chromatin status pattern of two families, wherein one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a methylation pattern of three families, wherein one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an chromatin status pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference chromatin status pattern can be obtained for fully pluripotent stem cells, as well as for cells that have more limited developmental potential (reduced pluripotency). Therefore, the present invention provides a method of assigning a chromatin status pattern of transposable elements to a fully pluripotent stem cell comprising: a) determining chromatin status of one or more families of transposable elements in a fully pluripotent stem cell and assigning the chromatin status pattern obtained from step a) to the cell.

The present invention also provides a method of assigning a chromatin status pattern of transposable elements to a pluripotent stem cell comprising: a) determining chromatin status of one or more families of transposable elements in a pluripotent stem cell and assigning the chromatin stauts pattern obtained from step a) to the cell.

Also provided by the present invention is a method of assigning a chromatin status pattern of transposable elements to a differentiated cell comprising: a) determining chromatin status of one or more families of transposable elements in a differentiated cell and assigning the chromatin status pattern obtained from step a) to the cell.

The present invention also provides a method of determining the developmental potential of a cell comprising: a) determining chromatin status of one or more families of transposable elements in a cell to obtain a chromatin status pattern; b) matching the chromatin status pattern of step a) with a known chromatin status pattern for a cell and c) determining the level of developmental potential of a cell based on matching of the expression pattern of a) with a known chromatin status pattern for a cell with a specific level of developmental potential.

In the methods of the present invention, the chromatin status pattern obtained from a sample cell taken from a subject can be obtained from outside sources, such as a testing laboratory or a commercial source. Therefore, the step of obtaining the chromatin status pattern can be performed by one skilled artisan and the step of comparing the chromatin status pattern can be performed by a second skilled artisan. Thus, the present invention provides a method of establishing the developmental potential of a cell or cells comprising: a) matching a test transposable element chromatin status pattern of a cell with a known chromatin status pattern for a cell with a specific level of developmental potential; and b) determining the level of developmental potential of the cell based on matching of the test chromatin status pattern with a known chromatin status pattern for a cell with a specific level of developmental potential.

For example, one of skill in the art can obtain a fertilized oocyte derived pluripotent stem cell and determine the chromatin status pattern of one or more transposable element families. By determining which transposable element families are methylated as well as which members of these transposable element families are methylated, one of skill in the art can assign this pattern to a fertilized oocyte derived pluripotent stem cell. This can be done for another stem cell with a more limited developmental potential than a fertilized oocyte, for example, a stem cell derived from a brain, such that a library of chromatin status patterns are readily available to not only to identify a cell with pluripotent potential but to determine the stage of pluripotency, i.e., level of developmental potential. Similarly, this can be done for stem cells derived from any tissue, or for oocytes in which a nucleus derived from a differentiated cell has been introduced to determine the degree to which that nucleus has reacquired pluripotency. By determining the chromatin status patterns of retrolements in cells with different stages of pluripotency, the skilled artisan can determine which transposable element families and which members of these families are markers of the level of pluripotency and developmental potential of cells.

Such libraries of chromatin status patterns are useful for determining the developmental potential of stem cells. For example, a nucleus from a fully differentiated cell from a patient with Parkinson's disease can be transplanted into an enucleated oocyte. Once the chromatin status pattern of putative stem cells descendent from this oocyte are determined according to the methods of the present invention, this chromatin status pattern can be compared to a library of chromatin status patterns to determine the level of pluripotency associated with the chromatin status pattern. Once this is determined, a decision can be made with regard to the potential of these stem cells to regenerate appropriate neural cells if implanted in the patient's brain. The present methods will also be useful in evaluating the effectiveness of various treatments in stimulating stem cells to develop or, conversely, to monitor the effectiveness of treatments to stimulate determined and/or differentiated cells to regain pluripotency. For example, a sample of partially or fully differentiated neural cells could be treated in vitro with oocyte cellular extracts or other chemicals, small molecules, peptides, growth factors etc. designed to reprogram differentiated cells or to increase pluripotency. Chromatin status patterns can be obtained from these treated cells and compared to chromatin status patterns pre-established to be characteristic of pluripotent stem cells. Since the skilled artisan will have reference patterns for the fully differentiated cell, as well as a fully pluripotent stem cell and stem cells of more limited pluripotency, changes in transposable element chromatin status after treatment can be monitored to determine if the treatment results in a transposable element chromatin status pattern that more closely resembles the chromatin status pattern for a pluripotent stem cell.

For example, if before treatment, certain families and members of these families are methylated, and after treatment, fewer families and/or members of these families are methylated, it can be said that this particular treatment is effective in increasing the developmental potential of the cell or in reprogramming the differentiated cell to become pluripotent. In some instances, effective treatments may involve decreasing the chromatin status of certain transposable elements and increasing the chromatin status of others. Therefore, once libraries of chromatin status patterns are established from untreated differentiated cells, one of skill in the art will know whether or not treatment is effective in a particular cell lineage by comparing the chromatin status pattern of a sample from samples of cells at different stages of treatment, with reference patterns established for the fully pluripotent stem cells. If a treatment is not successful in a particular cell lineage, the skilled artisan will recognize this by noting that the chromatin status pattern is not changing as expected, and other dosages, or treatments can be employed.

Also provided by the present invention is a method of identifying a cellular differentiation induction factor comprising: a) determining chromatins status of one or more families of transposable elements in a stem cell to obtain a first chromatin status pattern; b) administering a putative induction factor to the cell; c) determining the chromatin status of one or more families of transposable elements in the cell after administration of the putative induction factor to obtain a second chromatin status pattern; and d comparing the second chromatin status pattern with the first chromatin status pattern such that if there is a change in the second chromatin status pattern as compared to the first chromatin status pattern, the induction factor is a cellular differentiation induction factor.

Further provided by the present invention is a method of identifying a factor that increases the developmental potential of a cell comprising: a) determining chromatin status of one or more families of transposable elements in a differentiated cell to obtain a first chromatin status pattern; b) administering a putative factor that increases developmental potential to the cell; c) determining expression of one or more families of transposable elements in the cell after administration of the putative factor to obtain a second chromatin status pattern; and d) comparing the second chromatin status pattern with the first chromatin status pattern such that if there is a change in the second chromatin status pattern as compared to the first chromatin status pattern, the factor is effective in increasing the developmental potential of the cell.

In some instances, an effective treatment will result in fewer transposable elements being accessible to restriction enzymes in the second chromatin status pattern as compared to the first chromatin status pattern. In other instances, there may be more transposable elements accessible to restriction enzymes in the second pattern as compared to the first chromatin status pattern.

The chromatin status patterns of the present invention can also be used in combination with other diagnostic markers of genomic reprogramming, such as the loss of methylation of genes known to be characteristically and specifically expressed in specific types of differentiated cells (e.g the differentiated liver cell marker DDP IV-dipeptidyl peptidase—see Oh et al. 2000 Hepatocyte growth factor induces differentiation of adult rat bone marrow cells into a hepatocyte lineage in vitro. Biochem. Biophys. Res. Commun. 279: 500-504).

The present invention also provides a computer system comprising a) a database including records comprising a plurality of reference retroelement expression patterns, and associated developmental potential information; and b) a user interface capable of receiving a selection of one or more test retroelement expression patterns for use in determining matches between a test retroelement expression pattern and a reference retroelement expression pattern, and displaying the records associated with matching expression patterns. The computer systems of the present invention can also include a database including records comprising a plurality of reference methylation patterns, and associated developmental potential information, b) a user interface capable of receiving a selection of one or more test methylation patterns for use in determining matches between a test methylation pattern and the reference methylation pattern, and displaying the records associated with matching expression patterns. Also provided is a computer system comprising a) a database including records comprising a plurality of reference chromatin status patterns, and associated developmental potential information; and b) a user interface capable of receiving a selection of one or more test chromatin status patterns for use in determining matches between a test chromatin status pattern and a reference chromatin status pattern, and displaying the records associated with matching expression patterns.

It will be appreciated by those skilled in the art that expression patterns, methylation patterns and/or chromatin status patterns identified in cells as described by the present invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 expression patterns, methylation patterns and/or chromatin status patterns of the invention or patterns identified from cells.

Computer readable media include magnetically readable media, optically readable media, electronically readable media and magnetic/optical media. For example, the computer readable media may be a hard disc, a floppy disc, a magnetic tape, CD-ROM, DVD, RAM, or ROM as well as other types of other media known to those skilled in the art.

Embodiments of the present invention include systems, particularly computer systems which contain the sequence information described herein. As used herein, “a computer system” refers to the hardware components, software components, and data storage components used to store and/or analyze the expression patterns of the present invention or other expression patterns. The computer system preferably includes the computer readable media described above, and a processor for accessing and manipulating the data.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory, preferably implemented as RAM, and one or more data storage devices, such as a hard drive and/or other computer readable media having data recorded thereon. In some embodiments, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device.

In some embodiments, the computer system may further comprise an expression pattern comparer for comparing the expression pattern(s) stored on a computer readable medium to expression pattern(s) stored on a computer readable medium. An “expression pattern comparer” refers to one or more programs which are implemented on the computer system to compare a nucleotide sequence with other nucleotide sequences. Similarly, programs capable of comparing methylation status patterns and chromatin status patterns are also contemplated by the present invention.

This invention also provides for a computer program that correlates expression patterns with a particular level of developmental potential. Similarly, the present invention also provides a computer program that correlates methylation patterns with a particular level of developmental potential. Also provided is a computer program that correlates chromatin status with a particular level of developmental potential. The computer programs of this invention can optionally include treatment options for cells, such that one of skill in the art would be able to treat cells and modulate the developmental stage of the cell.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of assigning an expression pattern of transposable elements to the level of developmental potential of a cell comprising:
a) determining expression of one or more families of transposable elements; and
b) assigning the expression pattern obtained from step a) to the level of developmental potential of a cell.

2. The method of claim 1, wherein the cell is a fully pluripotent stem cell.

3. The method of claim 1, wherein the cell is a pluripotent stem cell.

4. The method of claim 1, wherein the cell is a differentiated cell.

5. The method of claim 1, wherein the expression pattern is determined by microarray analysis.

6. The method of claim 1, wherein one or more of the families of transposable elements are retroelement families.

7. The method of claim 1, wherein one or more of the families of transposable elements are DNA element families.

8. The method of claim 6, wherein one or more of the families of retroelements is selected from the group consisting of endogenous retroviruses (ERVs), a family of short interspersed nuclear elements (SINES) and a family of long interspersed nuclear elements (LINEs).

9. The method of claim 1, wherein expression of the transposable elements is measured by assaying for the mRNA transcribed from the genes or proteins translated from an mRNA transcribed from the genes.

10. The method of any of of claim 1, wherein expression of two or more families of transposable elements is determined and used to form the pattern of expression.

11. A method of determining the developmental potential of a stem cell comprising:
a) determining expression of one or more families of transposable elements in a stem cell to obtain an expression pattern;
b) matching the expression pattern of step a) with a known expression pattern for a cell at different stages of developmental potential ranging from a fully pluripotent stem cell to a fully differentiated cell; and
c) determining the developmental potential of the stem cell based on matching the expression pattern of a) with a known expression pattern for a cell at a specific developmental stage.

12. A method of identifying a cellular differentiation induction factor comprising:
a) determining expression of one or more families of transposable elements in a stem cell to obtain a first expression pattern;
b) administering a putative induction factor to the cell;
c) determining expression of one or more families of transposable elements in the cell after administration of the putative induction factor to obtain a second expression pattern; and
d) comparing the second expression pattern with the first expression pattern such that if transposable elements are differentially expressed in the second expression pattern as compared to the first expression pattern, the induction factor is a cellular differentiation induction factor.

13. A method of identifying a factor that increases the developmental potential of a cell comprising:
a) determining expression of one or more families of transposable elements in a cell to obtain a first expression pattern;
b) administering a putative factor that increases developmental potential to the cell;
c) determining expression of one or more families of transposable elements in the cell after administration of the putative factor to obtain a second expression pattern; and
d) comparing the second expression pattern with the first expression pattern such that if transposable elements are differentially expressed in the second expression pattern as compared to the first expression pattern, the factor is effective in increasing the developmental potential of the cell.

14. A method of assigning a methylation pattern of transposable elements to the level of developmental potential of a cell comprising:
a) determining methylation of one or more families of transposable elements; and
b) assigning the methylation pattern obtained from step a) to the level of developmental potential of a cell.

15. The method of claim 14, wherein the cell is a fully pluripotent stem cell.

16. The method of claim 14, wherein the cell is a pluripotent stem cell.

17. The method of claim 14 wherein the cell is a differentiated cell.

18. The method of claim 14, wherein the methylation pattern is determined by microarray analysis.

19. The method of claim 14, wherein one or more of the families of transposable elements are retroelement families.

20. The method of claim 14, wherein one or more of the families of transposable elements are DNA element families.

21. The method of claim 19, wherein one or more of the families of retroelements is selected from the group consisting of endogenous retroviruses (ERVs), a family of short interspersed nuclear elements (SINES) and a family of long interspersed nuclear elements (LINEs).

22. The method of claim 14, wherein methylation of two or more families of transposable elements is determined and used to form the methylation pattern

23. A method of determining the developmental potential of a stem cell comprising:
a) determining methylation of one or more families of transposable elements in a stem cell to obtain a methylation pattern;
b) matching the methyation pattern of step a) with a known methylation pattern for a cell at different stages of developmental potential ranging from a fully pluripotent stem cell to a fully differentiated cell and;
c) determining the developmental potential of the stem cell based on matching the methylation pattern of a) with a known methylation pattern for a cell at a specific developmental stage.

24. A method of identifying a cellular differentiation induction factor comprising:
a) determining methylation of one or more families of transposable elements in a stem cell to obtain a first methylation pattern;
b) administering a putative induction factor to the cell;
c) determining methylation of one or more families of transposable elements in the cell after administration of the putative induction factor to obtain a second methylation pattern; and
d) comparing the second methylation pattern with the first methylation pattern such that if transposable elements are differentially expressed in the second methylation pattern as compared to the first methylation pattern, the induction factor is a cellular differentiation induction factor.

25. A method of identifying a factor that increases the developmental potential of a cell comprising:
a) determining methylation of one or more families of transposable elements in a differentiated cell to obtain a first expression pattern;
b) administering a putative factor that increases developmental potential to the cell;
c) determining expression of one or more families of transposable elements in the cell after administration of the putative factor to obtain a second methylation pattern; and
d) comparing the second methylation pattern with the first methylation pattern such that if there is a change in the second methylation pattern as compared to the first methylation pattern, the factor is effective in increasing the developmental potential of the cell.

26. The method of any of claims 14, 23, 24 or 25, wherein methylation of the transposable element sequences is measured by contacting the methylated transposable element gene sequence with an antibody that specifically binds a methylated sequence.

27. The method of any of claims 14, 23, 24 or 25, wherein methylation of the transposable element sequences is measured by contacting the methylated transposable element gene sequence with an antibody that specifically binds a methylation complex protein associated with the methylated transposable element gene sequence.

28. The method of any of claims 14, 23, 24 or 25, wherein methylation of the transposable element genes is monitored by enzymatic means.

29. The method of any of claims 14, 23, 24 or 25, wherein methylation of the transposable element genes is monitored by microarray analysis.

30. The method of any of claims 14, 23, 24 or 25, wherein methylation of the transposable element genes is monitored by methylation-specific PCR.

31. The method of any of claims 14, 23, 24 or 25, wherein the methylation of two or more families of transposable elements is determined and used to form the methylation pattern.

32. A method of assigning a chromatin status pattern of transposable elements to the level of developmental potential of a cell comprising:
a) determining chromatin status of one or more families of transposable elements; and
b) assigning the chromatin status pattern obtained from step a) to the level of developmental potential of a cell.

33. The method of claim 32, wherein the cell is a fully pluripotent stem cell.

34. The method of claim 32, wherein the cell is a pluripotent stem cell.

35. The method of claim 32, wherein the cell is a differentiated cell.

36. The method of claim32, wherein the chromatin status pattern is determined by microarray analysis.

37. The method of claim 32, wherein one or more of the families of transposable elements are retroelement families.

38. The method of claim 32, wherein one or more of the families of transposable elements are DNA element families.

39. The method of claim 37, wherein one or more of the families of retroelements is selected from the group consisting of endogenous retroviruses (ERVs), a family of short interspersed nuclear elements (SINES) and a family of long interspersed nuclear elements (LINEs).

40. The method of claim 32, wherein the chromatin status of two or more families of transposable elements is determined and used to form the chromatin status pattern

41. A method of determining the developmental potential of a stem cell comprising:
a) determining chromatin status of one or more families of transposable elements in a stem cell to obtain a chromatin status pattern; and
b) matching the chromatin status pattern of step a) with a known chromatin status pattern for a cell at different stages of developmental potential ranging from a fully pluripotent stem cell to a fully differentiated cell and;
c) determining the developmental potential of the stem cell based on matching the chromatin status pattern of a) with a known chromatin status pattern for a cell at a specific developmental stage.

42. A method of identifying a cellular differentiation induction factor comprising:
a) determining chromatins status of one or more families of transposable elements in a stem cell to obtain a first chromatin status pattern;
b) administering a putative induction factor to the cell;
c) determining the chromatin status of one or more families of transposable elements in the cell after administration of the putative induction factor to obtain a second chromatin status pattern; and
d) comparing the second chromatin status pattern with the first chromatin status pattern such that if there is a change in the second chromatin status pattern as compared to the first chromatin status pattern, the induction factor is a cellular differentiation induction factor.

43. A method of identifying a factor that increases the developmental potential of a cell comprising:
a) determining chromatin status of one or more families of transposable elements in a differentiated cell to obtain a first chromatin status pattern;
b) administering a putative factor that increases developmental potential to the cell;
c) determining expression of one or more families of transposable elements in the cell after administration of the putative factor to obtain a second chromatin status pattern; and
d) comparing the second chromatin status pattern with the first chromatin status pattern such that if there is a change in the second chromatin status pattern as compared to the first chromatin status pattern, the factor is effective in increasing the developmental potential of the cell.

44. The method of any of claims 41-43 wherein chromatin status of the transposable elements genes is monitored by microarray analysis.

45. The method of any of claims 41-43 wherein the chromatin status of two or more families of transposable elements is determined and used to form the chromatin status pattern.
